SEQUENCE LISTING (136 sequences)

SEQ ID NO: 1 - protein, 5 aa, synthetic construct
  REGION 1..5: note = CDRH1 of hz2G10 antibody
DYYMY

SEQ ID NO: 2 - protein, 17 aa, synthetic construct
  REGION 1..17: note = CDRH2 of hz2G10 antibody
YINSGGGSTYYPDTVKG

SEQ ID NO: 3 - protein, 12 aa, synthetic construct
  REGION 1..12: note = CDRH3 of hz2G10 antibody
EALYDYDYAMDY

SEQ ID NO: 4 - protein, 16 aa, synthetic construct
  REGION 1..16: note = CDRL1 of hz2G10 antibody
KSSQSLLYSNGKTYLN

SEQ ID NO: 5 - protein, 7 aa, synthetic construct
  REGION 1..7: note = CDRL2 of hz2G10 antibody
LVSKLDS

SEQ ID NO: 6 - protein, 9 aa, synthetic construct
  REGION 1..9: note = CDRL3 of hz2G10 antibody
VQGTHFPLT

SEQ ID NO: 7 - protein, 5 aa, synthetic construct
  REGION 1..5: note = CDRH1 of hz39D2 antibody
NYGVN

SEQ ID NO: 8 - protein, 17 aa, synthetic construct
  REGION 1..17: note = CDRH2 of hz39D2 antibody
WINTHTGEPTYAEEFKG

SEQ ID NO: 9 - protein, 9 aa, synthetic construct
  REGION 1..9: note = CDRH3 of hz39D2 antibody
DDYYVRVDY

SEQ ID NO: 10 - protein, 11 aa, synthetic construct
  REGION 1..11: note = CDRL1 of hz39D2 antibody
KASQDINSYLS

SEQ ID NO: 11 - protein, 7 aa, synthetic construct
  REGION 1..7: note = CDRL2 of hz39D2 antibody
RANRLVD

SEQ ID NO: 12 - protein, 9 aa, synthetic construct
  REGION 1..9: note = CDRL3 of hz39D2 antibody
LQYDEFPWT

SEQ ID NO: 13 - protein, 5 aa, synthetic construct
  REGION 1..5: note = CDRH1 of 24D3 antibody
SCAMS

SEQ ID NO: 14 - protein, 17 aa, synthetic construct
  REGION 1..17: note = CDRH2 of 24D3 antibody
TISGGGSYTYYPDSVKG

SEQ ID NO: 15 - protein, 10 aa, synthetic construct
  REGION 1..10: note = CDRH3 of 24D3 antibody
HGGYESWFPY

SEQ ID NO: 16 - protein, 16 aa, synthetic construct
  REGION 1..16: note = CDRL1 of 24D3 antibody
RSSQSLVHSNGNTYLH

SEQ ID NO: 17 - protein, 7 aa, synthetic construct
  REGION 1..7: note = CDRL2 of 24D3 antibody
KVSNRFS

SEQ ID NO: 18 - protein, 10 aa, synthetic construct
  REGION 1..10: note = CDRL3 of 24D3 antibody
SQSTHVPPWT

SEQ ID NO: 19 - protein, 5 aa, synthetic construct
  REGION 1..5: note = CDRH1 of 1G3 antibody
DTYMH

SEQ ID NO: 20 - protein, 17 aa, synthetic construct
  REGION 1..17: note = CDRH2 of 1G3 antibody
RIDPANGYTRYDPNFQG

SEQ ID NO: 21 - protein, 10 aa, synthetic construct
  REGION 1..10: note = CDRH3 of 1G3 antibody
YYYGFYAMDY

SEQ ID NO: 22 - protein, 11 aa, synthetic construct
  REGION 1..11: note = CDRL1 of 1G3 antibody
KASQDVSTAVA

SEQ ID NO: 23 - protein, 7 aa, synthetic construct
  REGION 1..7: note = CDRL2 of 1G3 antibody
SASYRYT

SEQ ID NO: 24 - protein, 9 aa, synthetic construct
  REGION 1..9: note = CDRL3 of 1G3 antibody
QQHYSTPPT

SEQ ID NO: 25 - protein, 5 aa, synthetic construct
  REGION 1..5: note = CDRH1 of hz8G11 antibody
GYYMH

SEQ ID NO: 26 - protein, 17 aa, synthetic construct
  REGION 1..17: note = CDRH2 of hz8G11 antibody
HINPNNGGTSYNQKFKG

SEQ ID NO: 27 - protein, 6 aa, synthetic construct
  REGION 1..6: note = CDRH3 of hz8G11 antibody
EEAFAY

SEQ ID NO: 28 - protein, 11 aa, synthetic construct
  REGION 1..11: note = CDRL1 of hz8G11 antibody
RASQDISNYLN

SEQ ID NO: 29 - protein, 7 aa, synthetic construct
  REGION 1..7: note = CDRL2 of hz8G11 antibody
YTSRLHS

SEQ ID NO: 30 - protein, 9 aa, synthetic construct
  REGION 1..9: note = CDRL3 of hz8G11 antibody
QQGITPPWT

SEQ ID NO: 31 - protein, 121 aa, synthetic construct
  REGION 1..121: note = Amino acid sequence of hz2G10 heavy chain variable region
EVQLVESGGGLVQPGGSLRLSCAASGFTFSDYYMYWVRQAPGKGLEWVAYINSGGGSTYYPDTVKGRFTISRDNAKNSLYLQMNSLRAEDTAVYYCAREALYDYDYAMDYWGQGTTVTVSS

SEQ ID NO: 32 - DNA, 363 bp, synthetic construct
  misc_feature 1..363: note = Nucleic acid sequence of hz2G10 heavy chain variable region
  source 1..363: mol_type = other DNA
gaggtgcagttggtcgagtctggaggaggtctggtacagccagggggaagtctgagactgagctgcgccgcttctggttttacctttagcgattactatatgtattgggtaagacaggcacctggtaaaggtttggaatgggtggcctacataaactcgggcgggggcagcacctactacccggataccgtgaagggccgcttcaccatctcccgagacaacgcgaaaaattcattgtatctgcaaatgaactcacttagagctgaagatactgccgtttactactgcgccagagaagcactctatgactatgattacgctatggattactgggggcagggcacaaccgtcactgtttctagt

SEQ ID NO: 33 - protein, 451 aa, synthetic construct
  REGION 1..451: note = Amino acid sequence of hz2G10 heavy chain
EVQLVESGGGLVQPGGSLRLSCAASGFTFSDYYMYWVRQAPGKGLEWVAYINSGGGSTYYPDTVKGRFTISRDNAKNSLYLQMNSLRAEDTAVYYCAREALYDYDYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1353 bp, synthetic construct
  misc_feature 1..1353: note = Nucleic acid sequence of hz2G10 heavy chain
  source 1..1353: mol_type = other DNA
gaggtgcagttggtcgagtctggaggaggtctggtacagccagggggaagtctgagactgagctgcgccgcttctggttttacctttagcgattactatatgtattgggtaagacaggcacctggtaaaggtttggaatgggtggcctacataaactcgggcgggggcagcacctactacccggataccgtgaagggccgcttcaccatctcccgagacaacgcgaaaaattcattgtatctgcaaatgaactcacttagagctgaagatactgccgtttactactgcgccagagaagcactctatgactatgattacgctatggattactgggggcagggcacaaccgtcactgtttctagtgcctccaccaagggcccctccgtgttccctctggccccctccagcaagtccacctctggcggcacagccgccctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccctccagctctctgggcacccagacctacatctgtaacgtgaaccacaagccctccaacaccaaggtggacaagaaggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaatgccaagaccaagcccagagaggaacagtacaactccacctaccgggtggtgtctgtgctgaccgtgctgcaccaggactggctgaacggcaaagaatacaagtgcaaagtctccaacaaggccctgcctgcccccatcgaaaagaccatctccaaggccaagggccagccccgcgagccccaggtgtacaccctgccccctagccgggacgagctgaccaagaaccaggtgtccctgacctgtctggtgaaaggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 35 - protein, 112 aa, synthetic construct
  REGION 1..112: note = Amino acid sequence of hz2G10 light chain variable region
DIVMTQSPLSLSVTPGQPASISCKSSQSLLYSNGKTYLNWLLQKPGQSPQRLIYLVSKLDSGVPDRFSGSGSGTDFTLKISRVEAEDVGVYYCVQGTHFPLTFGGGTKVEIK

SEQ ID NO: 36 - DNA, 336 bp, synthetic construct
  misc_feature 1..336: note = Nucleic acid sequence of hz2G10 light chain variable region
  source 1..336: mol_type = other DNA
gacattgtcatgacgcagagccccctttcactcagcgtgactcccggtcagcccgccagcatttcctgtaaaagctctcagtcgctcctgtacagcaatggcaagacttatctgaattggctgttacagaaaccaggccaaagccctcaaaggcttatctacctggtgagtaagttagacagcggggtgcctgacagatttagcggatctggaagcgggaccgatttcacactaaaaatcagcagggttgaggcagaggacgtgggcgtgtattattgtgtgcagggcacacacttcccactcacattcgggggaggcacaaaggtggaaatcaag

SEQ ID NO: 37 - protein, 219 aa, synthetic construct
  REGION 1..219: note = Amino acid sequence of hz2G10 light chain
DIVMTQSPLSLSVTPGQPASISCKSSQSLLYSNGKTYLNWLLQKPGQSPQRLIYLVSKLDSGVPDRFSGSGSGTDFTLKISRVEAEDVGVYYCVQGTHFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 657 bp, synthetic construct
  misc_feature 1..657: note = Nucleic acid sequence of hz2G10 light chain
  source 1..657: mol_type = other DNA
gacattgtcatgacgcagagccccctttcactcagcgtgactcccggtcagcccgccagcatttcctgtaaaagctctcagtcgctcctgtacagcaatggcaagacttatctgaattggctgttacagaaaccaggccaaagccctcaaaggcttatctacctggtgagtaagttagacagcggggtgcctgacagatttagcggatctggaagcgggaccgatttcacactaaaaatcagcagggttgaggcagaggacgtgggcgtgtattattgtgtgcagggcacacacttcccactcacattcgggggaggcacaaaggtggaaatcaagcggaccgtggccgctccctccgtgttcatcttcccaccctccgacgagcagctgaagtccggcaccgccagcgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 39 - protein, 118 aa, synthetic construct
  REGION 1..118: note = Amino acid sequence of hz39D2 heavy chain variable region
QVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSS

SEQ ID NO: 40 - DNA, 354 bp, synthetic construct
  misc_feature 1..354: note = Nucleic acid sequence of hz39D2 heavy chain variable region
  source 1..354: mol_type = other DNA
caagtccaactcgtgcagtcaggatctgaactgaagaaacctggagcgagcgttaaggtttcctgcaaggccagcggctatacgttcactaactatggtgtcaactgggtgagacaggcacccggccagggcctggagtggatgggttggatcaatactcacacaggggaaccaacatatgctgaggagttcaaaggacggtttgtttttagtctggacacctccgtgtctaccgcctacctgcagatttccagccttaaagcagaggacactgctgtatactactgtgccagagacgattactatgtgagggtggattactgggggcaggggaccaccgtgacagtctcaagt

SEQ ID NO: 41 - protein, 448 aa, synthetic construct
  REGION 1..448: note = Amino acid sequence of hz39D2 heavy chain
QVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 1344 bp, synthetic construct
  misc_feature 1..1344: note = Nucleic acid sequence of hz39D2 heavy chain
  source 1..1344: mol_type = other DNA
caagtccaactcgtgcagtcaggatctgaactgaagaaacctggagcgagcgttaaggtttcctgcaaggccagcggctatacgttcactaactatggtgtcaactgggtgagacaggcacccggccagggcctggagtggatgggttggatcaatactcacacaggggaaccaacatatgctgaggagttcaaaggacggtttgtttttagtctggacacctccgtgtctaccgcctacctgcagatttccagccttaaagcagaggacactgctgtatactactgtgccagagacgattactatgtgagggtggattactgggggcaggggaccaccgtgacagtctcaagtgcctccaccaagggcccctccgtgttccctctggccccctccagcaagtccacctctggcggcacagccgccctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccctccagctctctgggcacccagacctacatctgtaacgtgaaccacaagccctccaacaccaaggtggacaagaaggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaatgccaagaccaagcccagagaggaacagtacaactccacctaccgggtggtgtctgtgctgaccgtgctgcaccaggactggctgaacggcaaagaatacaagtgcaaagtctccaacaaggccctgcctgcccccatcgaaaagaccatctccaaggccaagggccagccccgcgagccccaggtgtacaccctgccccctagccgggacgagctgaccaagaaccaggtgtccctgacctgtctggtgaaaggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 43 - protein, 107 aa, synthetic construct
  REGION 1..107: note = Amino acid sequence of hz39D2 light chain variable region
DIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIK

SEQ ID NO: 44 - DNA, 321 bp, synthetic construct
  misc_feature 1..321: note = Nucleic acid sequence of hz39D2 light chain variable region
  source 1..321: mol_type = other DNA
gacattcaaatgacacagtctcccagctcccttagtgcttcggtgggcgatcgggtgaccataacatgcaaggcctcacaggacatcaacagctatctctcatggtttcagcagaagccaggaaaagcacctaaaacgttgatctacagggccaatcgcctcgttgacggagtcccctccagattcagcgggagtgggtctggtcaggattatactctgaccatctcctctctgcagcctgaagactttgccacttactactgtctgcaatacgatgagttcccatggaccttcggccagggcaccaaggtggagattaaa

SEQ ID NO: 45 - protein, 214 aa, synthetic construct
  REGION 1..214: note = Amino acid sequence of hz39D2 light chain
DIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 642 bp, synthetic construct
  misc_feature 1..642: note = Nucleic acid sequence of hz39D2 light chain
  source 1..642: mol_type = other DNA
gacattcaaatgacacagtctcccagctcccttagtgcttcggtgggcgatcgggtgaccataacatgcaaggcctcacaggacatcaacagctatctctcatggtttcagcagaagccaggaaaagcacctaaaacgttgatctacagggccaatcgcctcgttgacggagtcccctccagattcagcgggagtgggtctggtcaggattatactctgaccatctcctctctgcagcctgaagactttgccacttactactgtctgcaatacgatgagttcccatggaccttcggccagggcaccaaggtggagattaaacggaccgtggccgctccctccgtgttcatcttcccaccctccgacgagcagctgaagtccggcaccgccagcgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 47 - protein, 119 aa, synthetic construct
  REGION 1..119: note = Amino acid sequence of 24D3 heavy chain variable region
EVKLVESGGGLVKPGGSLKLSCAASGFTFSSCAMSWVRQTPEKRLEWVATISGGGSYTYYPDSVKGRFTISRDNAKNTLYLQMSSLRSEDTAMYYCARHGGYESWFPYWGQGTLVTVSA

SEQ ID NO: 48 - DNA, 357 bp, synthetic construct
  misc_feature 1..357: note = Nucleic acid sequence of 24D3 heavy chain variable region
  source 1..357: mol_type = other DNA
gaggtgaagctggtggagtctgggggaggcttagtgaagcctggagggtccctgaaactctcctgtgcagcctctggattcactttcagtagctgtgccatgtcttgggtccgccagactccggagaagaggctggagtgggtcgcaaccattagtggtggtggtagttacacctactatccagacagtgtgaaggggcgattcaccatctccagagacaatgccaagaacaccctgtacctgcaaatgagcagtctgaggtctgaggacacggccatgtattactgtgcaagacatggcgggtacgagtcctggtttccttactggggccaagggactctggtcactgtctctgca

SEQ ID NO: 49 - protein, 449 aa, synthetic construct
  REGION 1..449: note = Amino acid sequence of 24D3 heavy chain
EVKLVESGGGLVKPGGSLKLSCAASGFTFSSCAMSWVRQTPEKRLEWVATISGGGSYTYYPDSVKGRFTISRDNAKNTLYLQMSSLRSEDTAMYYCARHGGYESWFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 1347 bp, synthetic construct
  misc_feature 1..1347: note = Nucleic acid sequence of 24D3 heavy chain
  source 1..1347: mol_type = other DNA
gaggtgaagctggtggagtctgggggaggcttagtgaagcctggagggtccctgaaactctcctgtgcagcctctggattcactttcagtagctgtgccatgtcttgggtccgccagactccggagaagaggctggagtgggtcgcaaccattagtggtggtggtagttacacctactatccagacagtgtgaaggggcgattcaccatctccagagacaatgccaagaacaccctgtacctgcaaatgagcagtctgaggtctgaggacacggccatgtattactgtgcaagacatggcgggtacgagtcctggtttccttactggggccaagggactctggtcactgtctctgcagcctccaccaagggcccctccgtgttccctctggccccctccagcaagtccacctctggcggcacagccgccctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccctccagctctctgggcacccagacctacatctgtaacgtgaaccacaagccctccaacaccaaggtggacaagaaggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaatgccaagaccaagcccagagaggaacagtacaactccacctaccgggtggtgtctgtgctgaccgtgctgcaccaggactggctgaacggcaaagaatacaagtgcaaagtctccaacaaggccctgcctgcccccatcgaaaagaccatctccaaggccaagggccagccccgcgagccccaggtgtacaccctgccccctagccgggacgagctgaccaagaaccaggtgtccctgacctgtctggtgaaaggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 51 - protein, 113 aa, synthetic construct
  REGION 1..113: note = Amino acid sequence of 24D3 light chain variable region
DIVMTQSPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIK

SEQ ID NO: 52 - DNA, 339 bp, synthetic construct
  misc_feature 1..339: note = Nucleic acid sequence of 24D3 light chain variable region
  source 1..339: mol_type = other DNA
gatattgtgatgacccagtctccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggttccagacaggttcagtggcagtggatcagggacagatttcacactcaagatcagcagagtggaggctgaggatctgggagtttatttctgctctcaaagtacacatgttcctccgtggacgttcggtggagggaccaagctggaaatcaaa

SEQ ID NO: 53 - protein, 220 aa, synthetic construct
  REGION 1..220: note = Amino acid sequence of 24D3 light chain
DIVMTQSPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - DNA, 660 bp, synthetic construct
  misc_feature 1..660: note = Nucleic acid sequence of 24D3 light chain
  source 1..660: mol_type = other DNA
gatattgtgatgacccagtctccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggttccagacaggttcagtggcagtggatcagggacagatttcacactcaagatcagcagagtggaggctgaggatctgggagtttatttctgctctcaaagtacacatgttcctccgtggacgttcggtggagggaccaagctggaaatcaaacggaccgtggccgctccctccgtgttcatcttcccaccctccgacgagcagctgaagtccggcaccgccagcgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 55 - protein, 119 aa, synthetic construct
  REGION 1..119: note = Amino acid sequence of 1G3 heavy chain variable region
EVQLQQSGAELVKPGASVKLSCTASDFNIVDTYMHWVKQRPEQGLEWIGRIDPANGYTRYDPNFQGKATITADTSSNTAYLQLSSLTSEDTAVYYCARYYYGFYAMDYWGQGTTVTVSS

SEQ ID NO: 56 - DNA, 357 bp, synthetic construct
  misc_feature 1..357: note = Nucleic acid sequence of 1G3 heavy chain variable region
  source 1..357: mol_type = other DNA
gaggttcagctgcagcagtctggggcagagcttgtgaagccaggggcctcagtcaagttgtcctgcacagcttctgacttcaacattgtagacacctatatgcactgggtgaagcagaggcctgaacagggcctggagtggattggaaggattgatcctgcgaatggttatactagatatgacccgaacttccagggcaaggccactataacagcagacacatcctccaacacagcctacctgcagctcagcagcctgacatctgaggacactgccgtctattactgtgcccgttattactacggcttctatgctatggactactggggtcaaggaaccacggtcaccgtctcctca

SEQ ID NO: 57 - protein, 449 aa, synthetic construct
  REGION 1..449: note = Amino acid sequence of 1G3 heavy chain
EVQLQQSGAELVKPGASVKLSCTASDFNIVDTYMHWVKQRPEQGLEWIGRIDPANGYTRYDPNFQGKATITADTSSNTAYLQLSSLTSEDTAVYYCARYYYGFYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - DNA, 1347 bp, synthetic construct
  misc_feature 1..1347: note = Nucleic acid sequence of 1G3 heavy chain
  source 1..1347: mol_type = other DNA
gaggttcagctgcagcagtctggggcagagcttgtgaagccaggggcctcagtcaagttgtcctgcacagcttctgacttcaacattgtagacacctatatgcactgggtgaagcagaggcctgaacagggcctggagtggattggaaggattgatcctgcgaatggttatactagatatgacccgaacttccagggcaaggccactataacagcagacacatcctccaacacagcctacctgcagctcagcagcctgacatctgaggacactgccgtctattactgtgcccgttattactacggcttctatgctatggactactggggtcaaggaaccacggtcaccgtctcctcagcctccaccaagggcccctccgtgttccctctggccccctccagcaagtccacctctggcggcacagccgccctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccctccagctctctgggcacccagacctacatctgtaacgtgaaccacaagccctccaacaccaaggtggacaagaaggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaatgccaagaccaagcccagagaggaacagtacaactccacctaccgggtggtgtctgtgctgaccgtgctgcaccaggactggctgaacggcaaagaatacaagtgcaaagtctccaacaaggccctgcctgcccccatcgaaaagaccatctccaaggccaagggccagccccgcgagccccaggtgtacaccctgccccctagccgggacgagctgaccaagaaccaggtgtccctgacctgtctggtgaaaggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 59 - protein, 107 aa, synthetic construct
  REGION 1..107: note = Amino acid sequence of 1G3 light chain variable region
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPTFGGGTKLELK

SEQ ID NO: 60 - DNA, 321 bp, synthetic construct
  misc_feature 1..321: note = Nucleic acid sequence of 1G3 light chain variable region
  source 1..321: mol_type = other DNA
gatattgtgatgacgcagtctcacaaattcatgtccacatcagtaggagacagggtcagcatcacctgcaaggccagtcaggatgtgagtactgctgtagcctggtatcaacagaaaccaggacaatctcctaaactactgatttactcggcatcctaccggtacactggagtccctgatcgcttcactggcagtggatctgggacggatttcactttcaccatcagcagtgtgcaggctgaagacctggcagtttattactgtcagcaacattatagtactcctcccacgttcggaggggggaccaagctggagctgaaa

SEQ ID NO: 61 - protein, 214 aa, synthetic construct
  REGION 1..214: note = Amino acid sequence of 1G3 light chain
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 642 bp, synthetic construct
  misc_feature 1..642: note = Nucleic acid sequence of 1G3 light chain
  source 1..642: mol_type = other DNA
gatattgtgatgacgcagtctcacaaattcatgtccacatcagtaggagacagggtcagcatcacctgcaaggccagtcaggatgtgagtactgctgtagcctggtatcaacagaaaccaggacaatctcctaaactactgatttactcggcatcctaccggtacactggagtccctgatcgcttcactggcagtggatctgggacggatttcactttcaccatcagcagtgtgcaggctgaagacctggcagtttattactgtcagcaacattatagtactcctcccacgttcggaggggggaccaagctggagctgaaacggaccgtggccgctccctccgtgttcatcttcccaccctccgacgagcagctgaagtccggcaccgccagcgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 63 - protein, 115 aa, synthetic construct
  REGION 1..115: note = Amino acid sequence of hz8G11 heavy chain variable region
QVQLVQSGQEVKKPGASVKVSCKASGYSFTGYYMHWVRQAPGQGLEWIGHINPNNGGTSYNQKFKGRTTLTVDKSISTAYMELSRLRSDDTAVYYCAREEAFAYWGQGTLVTVSS

SEQ ID NO: 64 - DNA, 345 bp, synthetic construct
  misc_feature 1..345: note = Nucleic acid sequence of hz8G11 heavy chain variable region
  source 1..345: mol_type = other DNA
caggtacagctagtgcagagcggccaggaagtaaagaagccaggcgcctctgttaaggtgtcatgtaaggccagcggttacagcttcactggctattacatgcactgggtccggcaggcacccggacaagggctggaatggataggtcacattaatccaaacaatggcggtaccagttataaccagaaatttaaggggaggacaaccctgacagttgataaatccatcagtacagcatatatggagctcagcagactgagaagcgacgatactgctgtgtactactgcgcgcgggaggaggctttcgcctactggggccaagggaccttagtgactgtctcatca

SEQ ID NO: 65 - protein, 445 aa, synthetic construct
  REGION 1..445: note = Amino acid sequence of hz8G11 heavy chain
QVQLVQSGQEVKKPGASVKVSCKASGYSFTGYYMHWVRQAPGQGLEWIGHINPNNGGTSYNQKFKGRTTLTVDKSISTAYMELSRLRSDDTAVYYCAREEAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - DNA, 1335 bp, synthetic construct
  misc_feature 1..1335: note = Nucleic acid sequence of hz8G11 heavy chain
  source 1..1335: mol_type = other DNA
caggtacagctagtgcagagcggccaggaagtaaagaagccaggcgcctctgttaaggtgtcatgtaaggccagcggttacagcttcactggctattacatgcactgggtccggcaggcacccggacaagggctggaatggataggtcacattaatccaaacaatggcggtaccagttataaccagaaatttaaggggaggacaaccctgacagttgataaatccatcagtacagcatatatggagctcagcagactgagaagcgacgatactgctgtgtactactgcgcgcgggaggaggctttcgcctactggggccaagggaccttagtgactgtctcatcagcctccaccaagggcccctccgtgttccctctggccccctccagcaagtccacctctggcggcacagccgccctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccctccagctctctgggcacccagacctacatctgtaacgtgaaccacaagccctccaacaccaaggtggacaagaaggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaatgccaagaccaagcccagagaggaacagtacaactccacctaccgggtggtgtctgtgctgaccgtgctgcaccaggactggctgaacggcaaagaatacaagtgcaaagtctccaacaaggccctgcctgcccccatcgaaaagaccatctccaaggccaagggccagccccgcgagccccaggtgtacaccctgccccctagccgggacgagctgaccaagaaccaggtgtccctgacctgtctggtgaaaggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 67 - protein, 107 aa, synthetic construct
  REGION 1..107: note = Amino acid sequence of hz8G11 light chain variable region
DIQMTQSPSSLSASVGDRVTISCRASQDISNYLNWYQQKPGKAPKLLIYYTSRLHSGVPSRFSGSGSGTDFSLTISSLQPEDIATYYCQQGITPPWTFGGGTKVEIK

SEQ ID NO: 68 - DNA, 321 bp, synthetic construct
  misc_feature 1..321: note = Nucleic acid sequence of hz8G11 light chain variable region
  source 1..321: mol_type = other DNA
gacatacagatgacgcagagccctagttcactgtctgcctccgtcggcgacagagtgacgatcagctgccgagccagccaagatattagtaactacctcaattggtaccagcagaaacctggaaaagcacccaagcttttgatctattacaccagcaggctgcatagcggagtgccgagcagattttcgggttctggcagcggcaccgatttctctctgactatcagtagcctgcaacccgaagacattgctacatattattgtcagcagggaatcacccctccatggacatttggagggggaacaaaggtggagattaaa

SEQ ID NO: 69 - protein, 214 aa, synthetic construct
  REGION 1..214: note = Amino acid sequence of hz8G11 light chain
DIQMTQSPSSLSASVGDRVTISCRASQDISNYLNWYQQKPGKAPKLLIYYTSRLHSGVPSRFSGSGSGTDFSLTISSLQPEDIATYYCQQGITPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - DNA, 642 bp, synthetic construct
  misc_feature 1..642: note = Nucleic acid sequence of hz8G11 light chain
  source 1..642: mol_type = other DNA
gacatacagatgacgcagagccctagttcactgtctgcctccgtcggcgacagagtgacgatcagctgccgagccagccaagatattagtaactacctcaattggtaccagcagaaacctggaaaagcacccaagcttttgatctattacaccagcaggctgcatagcggagtgccgagcagattttcgggttctggcagcggcaccgatttctctctgactatcagtagcctgcaacccgaagacattgctacatattattgtcagcagggaatcacccctccatggacatttggagggggaacaaaggtggagattaaacggaccgtggccgctccctccgtgttcatcttcccaccctccgacgagcagctgaagtccggcaccgccagcgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 71 - protein, 9 aa, synthetic construct
  REGION 1..9: note = CDRH3 of hz39D2.14 antibody
DEYYVRTDY

SEQ ID NO: 72 - protein, 9 aa, synthetic construct
  REGION 1..9: note = CDRH3 of hz39D2.22 and hz39D2.23 antibody
DEYYVRVDY

SEQ ID NO: 73 - protein, 9 aa, synthetic construct
  REGION 1..9: note = CDRL3 of hz39D2.22 antibody
LELDEFPWT

SEQ ID NO: 74 - protein, 9 aa, synthetic construct
  REGION 1..9: note = CDRL3 of hz39D2.23 antibody
LQLDEFPWT

SEQ ID NO: 75 - protein, 121 aa, synthetic construct
  REGION 1..121: note = Amino acid sequence of 2G10 heavy chain variable region
EVKLLESGGGLVQPGGSLKLSCATSGFTFSDYYMYWVRQTPEMRLEWVAYINSGGGSTYYPDTVKGRFTISRDNAKNTLYLQMSRLKSEDTAMYYCAREALYDYDYAMDYWGQGTTVTVSS

SEQ ID NO: 76 - DNA, 363 bp, synthetic construct
  misc_feature 1..363: note = Nucleic acid sequence of 2G10 heavy chain variable region
  source 1..363: mol_type = other DNA
gaggtgaagcttctcgagtctgggggaggcttagtgcagcctggagggtccctgaaactctcctgtgcaacctctggattcactttcagtgactattacatgtattgggttcgccagactccagagatgaggctggagtgggtcgcatatattaatagtggtggtggtagcacctattatccagacactgtaaagggccgattcaccatctccagagacaatgccaagaacaccctgtacctgcaaatgagccgtctgaagtctgaggacacagccatgtattactgtgcaagagaggccctctatgattacgactatgctatggactactggggtcaaggaaccacggtcaccgtctcctca

SEQ ID NO: 77 - protein, 112 aa, synthetic construct
  REGION 1..112: note = Amino acid sequence of 2G10 light chain variable region
DIVMTQSPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPLTFGAGTKLELK

SEQ ID NO: 78 - DNA, 336 bp, synthetic construct
  misc_feature 1..336: note = Nucleic acid sequence of 2G10 light chain variable region
  source 1..336: mol_type = other DNA
gatattgtgatgacccagtctccactcactttgtcggttaccattggacaaccagcctctatctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtgggtcaggaacagattttacactgaaaatcagcagagtggaggctgaggatttgggagtttattactgcgtgcaaggtacacattttccgctcacgttcggtgctgggaccaagctggagctgaaa

SEQ ID NO: 79 - protein, 115 aa, synthetic construct
  REGION 1..115: note = Amino acid sequence of 8G11 heavy chain variable region
EVQLQQSGPDLVKPGTSVKISCKASGYSFTGYYMHWVKQSHGKSLEWIGHINPNNGGTSYNQKFKGKTILTVDKSSSTAFMELRSLTSEDSAVYYCAREEAFAYWGQGTLVTVSA

SEQ ID NO: 80 - DNA, 345 bp, synthetic construct
  misc_feature 1..345: note = Nucleic acid sequence of 8G11 heavy chain variable region
  source 1..345: mol_type = other DNA
gaggtgcaacttcagcagtctggacctgacctggtgaagcctgggacttcagtgaagatatcctgcaaggcttctggttactcattcactggctactacatgcactgggtgaagcagagccatggaaagagccttgagtggattggacatattaatcctaacaatggtggtactagctacaaccagaagttcaagggcaagaccatattaactgtggacaagtcttccagcacagccttcatggagctccgcagcctgacatctgaggactctgcggtctattactgtgcaagagaagaagcctttgcttactggggccaagggactctggtcactgtctctgca

SEQ ID NO: 81 - protein, 107 aa, synthetic construct
  REGION 1..107: note = Amino acid sequence of 8G11 light chain variable region
DIVMTQSTSSLSASLGDRVTISCRASQDISNYLNWYQQKPDGTVKLLIYYTSRLHSGVPSRFSGSGSGTDFSLTISNVEQEDIATYFCQQGITPPWTFGGGTKLELK

SEQ ID NO: 82 - DNA, 321 bp, synthetic construct
  misc_feature 1..321: note = Nucleic acid sequence of 8G11 light chain variable region
  source 1..321: mol_type = other DNA
gatattgtgatgacccagtctacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagatttttctctcaccattagcaacgtggagcaagaagacattgccacttacttttgccaacagggtattacgcctccgtggacgttcggtggagggaccaagctggagctgaaa

SEQ ID NO: 83 - protein, 118 aa, synthetic construct
  REGION 1..118: note = Amino acid sequence of 39D2 heavy chain variable region
EVQLQQSGPELKKPGETVKISCKASGYTFTNYGVNWVKQAPGKGLKWMGWINTHTGEPTYAEEFKGRFAFSLETSASTAYLQINNLKNEDTATYFCARDDYYVRVDYWGQGTTLTVSS

SEQ ID NO: 84 - DNA, 354 bp, synthetic construct
  misc_feature 1..354: note = Nucleic acid sequence of 39D2 heavy chain variable region
  source 1..354: mol_type = other DNA
gaggttcagctgcagcagtctggacctgagctgaagaagcctggagagacagtcaagatctcctgcaaggcttctgggtataccttcacaaactatggagtgaattgggtgaagcaggctccaggaaagggtttaaagtggatgggctggataaacacccacactggagagccaacatatgctgaagagttcaagggacggtttgccttctctttggaaacctctgccagcactgcctatttgcagatcaacaacctcaaaaatgaggacacggctacatatttctgtgcaagagatgattactacgtaagggtagactactggggccaaggcaccactctcacagtctcctca

SEQ ID NO: 85 - protein, 107 aa, synthetic construct
  REGION 1..107: note = Amino acid sequence of 39D2 light chain variable region
DIVMTQSPSSMYASLGERVTITCKASQDINSYLSWFQQKPGKSPKTLIYRANRLVDGVPSRFSGSGSGQDYSLTISSLEYEDMGIYYCLQYDEFPWTFGGGTKLELK

SEQ ID NO: 86 - DNA, 321 bp, synthetic construct
  misc_feature 1..321: note = Nucleic acid sequence of 39D2 light chain variable region
  source 1..321: mol_type = other DNA
gatattgtaatgacccagtctccatcttccatgtatgcatccctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgtggacgttcggtggagggaccaagctggagctgaaa

SEQ ID NO: 87 - protein, 118 aa, synthetic construct
  REGION 1..118: note = Amino acid sequence of hz39D2.14 heavy chain variable region
QVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDEYYVRTDYWGQGTTVTVSS

SEQ ID NO: 88 - DNA, 354 bp, synthetic construct
  misc_feature 1..354: note = Nucleic acid sequence of hz39D2.14 heavy chain variable region
  source 1..354: mol_type = other DNA
caagtccaactcgtgcagtcaggatctgaactgaagaaacctggagcgagcgttaaggtttcctgcaaggccagcggctatacgttcactaactatggtgtcaactgggtgagacaggcacccggccagggcctggagtggatgggttggatcaatactcacacaggggaaccaacatatgctgaggagttcaaaggacggtttgtttttagtctggacacctccgtgtctaccgcctacctgcagatttccagccttaaagcagaggacactgctgtatactactgtgccagagacgagtactatgtgaggaccgattactgggggcaggggaccaccgtgacagtctcaagt

SEQ ID NO: 89 - protein, 448 aa, synthetic construct
  REGION 1..448: note = Amino acid sequence of hz39D2.14 heavy chain
QVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDEYYVRTDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - DNA, 1344 bp, synthetic construct
  misc_feature 1..1344: note = Nucleic acid sequence of hz39D2.14 heavy chain
  source 1..1344: mol_type = other DNA
caagtccaactcgtgcagtcaggatctgaactgaagaaacctggagcgagcgttaaggtttcctgcaaggccagcggctatacgttcactaactatggtgtcaactgggtgagacaggcacccggccagggcctggagtggatgggttggatcaatactcacacaggggaaccaacatatgctgaggagttcaaaggacggtttgtttttagtctggacacctccgtgtctaccgcctacctgcagatttccagccttaaagcagaggacactgctgtatactactgtgccagagacgagtactatgtgaggaccgattactgggggcaggggaccaccgtgacagtctcaagtgcctccaccaagggcccctccgtgttccctctggccccctccagcaagtccacctctggcggcacagccgccctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccctccagctctctgggcacccagacctacatctgtaacgtgaaccacaagccctccaacaccaaggtggacaagaaggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaatgccaagaccaagcccagagaggaacagtacaactccacctaccgggtggtgtctgtgctgaccgtgctgcaccaggactggctgaacggcaaagaatacaagtgcaaagtctccaacaaggccctgcctgcccccatcgaaaagaccatctccaaggccaagggccagccccgcgagccccaggtgtacaccctgccccctagccgggacgagctgaccaagaaccaggtgtccctgacctgtctggtgaaaggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 91 - protein, 107 aa, synthetic construct; identical to SEQ ID NO: 43
  REGION 1..107: note = Amino acid sequence of hz39D2.14 light chain variable region

SEQ ID NO: 92 - DNA, 321 bp, synthetic construct
  misc_feature 1..321: note = Nucleic acid sequence of hz39D2.14 light chain variable region
  source 1..321: mol_type = other DNA
cggaccgtggccgctccctccgtgttcatcttcccaccctccgacgagcagctgaagtccggcaccgccagcgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 93 - protein, 214 aa, synthetic construct; identical to SEQ ID NO: 45
  REGION 1..214: note = Amino acid sequence of hz39D2.14 light chain

SEQ ID NO: 94 - DNA, 642 bp, synthetic construct; identical to SEQ ID NO: 46
  misc_feature 1..642: note = Nucleic acid sequence of hz39D2.14 light chain
  source 1..642: mol_type = other DNA

SEQ ID NO: 95 - protein, 118 aa, synthetic construct
  REGION 1..118: note = Amino acid sequence of hz39D2.22 heavy chain variable region
QVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDEYYVRVDYWGQGTTVTVSS

SEQ ID NO: 96 - DNA, 354 bp, synthetic construct
  misc_feature 1..354: note = Nucleic acid sequence of hz39D2.22 heavy chain variable region
  source 1..354: mol_type = other DNA
caagtccaactcgtgcagtcaggatctgaactgaagaaacctggagcgagcgttaaggtttcctgcaaggccagcggctatacgttcactaactatggtgtcaactgggtgagacaggcacccggccagggcctggagtggatgggttggatcaatactcacacaggggaaccaacatatgctgaggagttcaaaggacggtttgtttttagtctggacacctccgtgtctaccgcctacctgcagatttccagccttaaagcagaggacactgctgtatactactgtgccagagacgagtactatgtgagggtggattactgggggcaggggaccaccgtgacagtctcaagt

SEQ ID NO: 97 - protein, 448 aa, synthetic construct
  REGION 1..448: note = Amino acid sequence of hz39D2.22 heavy chain
QVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDEYYVRVDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - DNA, 1344 bp, synthetic construct
  misc_feature 1..1344: note = Nucleic acid sequence of hz39D2.22 heavy chain
  source 1..1344: mol_type = other DNA
caagtccaactcgtgcagtcaggatctgaactgaagaaacctggagcgagcgttaaggtttcctgcaaggccagcggctatacgttcactaactatggtgtcaactgggtgagacaggcacccggccagggcctggagtggatgggttggatcaatactcacacaggggaaccaacatatgctgaggagttcaaaggacggtttgtttttagtctggacacctccgtgtctaccgcctacctgcagatttccagccttaaagcagaggacactgctgtatactactgtgccagagacgagtactatgtgagggtggattactgggggcaggggaccaccgtgacagtctcaagtgcctccaccaagggcccctccgtgttccctctggccccctccagcaagtccacctctggcggcacagccgccctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccctccagctctctgggcacccagacctacatctgtaacgtgaaccacaagccctccaacaccaaggtggacaagaaggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaatgccaagaccaagcccagagaggaacagtacaactccacctaccgggtggtgtctgtgctgaccgtgctgcaccaggactggctgaacggcaaagaatacaagtgcaaagtctccaacaaggccctgcctgcccccatcgaaaagaccatctccaaggccaagggccagccccgcgagccccaggtgtacaccctgccccctagccgggacgagctgaccaagaaccaggtgtccctgacctgtctggtgaaaggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 99 - protein, 107 aa, synthetic construct
  REGION 1..107: note = Amino acid sequence of hz39D2.22 light chain variable region
DIQMTQSPSSLSASVGDRVTITCKASQDINSYLSWFQQKPGKAPKTLIYRANRLVDGVPSRFSGSGSGQDYTLTISSLQPEDFATYYCLELDEFPWTFGQGTKVEIK

SEQ ID NO: 100 - DNA, 321 bp, synthetic construct
  misc_feature 1..321: note = Nucleic acid sequence of hz39D2.22 light chain variable region
  source 1..321: mol_type = other DNA
gacattcaaatgacacagtctcccagctcccttagtgcttcggtgggcgatcgggtgaccataacatgcaaggcctcacaggacatcaacagctatctctcatggtttcagcagaagccaggaaaagcacctaaaacgttgatctacagggccaatcgcctcgttgacggagtcccctccagattcagcgggagtgggtctggtcaggattatactctgaccatctcctctctgcagcctgaagactttgccacttactactgtctggagctcgatgagttcccatggaccttcggccagggcaccaaggtggagattaaa

SEQ ID NO: 101 - protein, 214 aa, synthetic construct
  REGION 1..214: note = Amino acid sequence of hz39D2.22 light chain
DIQMTQSPSSLSASVGDRVTITCKASQDINSYLSWFQQKPGKAPKTLIYRANRLVDGVPSRFSGSGSGQDYTLTISSLQPEDFATYYCLELDEFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 642 bp, synthetic construct
  misc_feature 1..642: note = Nucleic acid sequence of hz39D2.22 light chain
  source 1..642: mol_type = other DNA
gacattcaaatgacacagtctcccagctcccttagtgcttcggtgggcgatcgggtgaccataacatgcaaggcctcacaggacatcaacagctatctctcatggtttcagcagaagccaggaaaagcacctaaaacgttgatctacagggccaatcgcctcgttgacggagtcccctccagattcagcgggagtgggtctggtcaggattatactctgaccatctcctctctgcagcctgaagactttgccacttactactgtctggagctcgatgagttcccatggaccttcggccagggcaccaaggtggagattaaacggaccgtggccgctccctccgtgttcatcttcccaccctccgacgagcagctgaagtccggcaccgccagcgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 103 - protein, 118 aa, synthetic construct; identical to SEQ ID NO: 95
  REGION 1..118: note = Amino acid sequence of hz39D2.23 heavy chain variable region

SEQ ID NO: 104 - DNA, 354 bp, synthetic construct; identical to SEQ ID NO: 96
  misc_feature 1..354: note = Nucleic acid sequence of hz39D2.23 heavy chain variable region
  source 1..354: mol_type = other DNA

SEQ ID NO: 105 - protein, 448 aa, synthetic construct; identical to SEQ ID NO: 97
  REGION 1..448: note = Amino acid sequence of hz39D2.23 heavy chain

SEQ ID NO: 106 - DNA, 1344 bp, synthetic construct; identical to SEQ ID NO: 98
  misc_feature 1..1344: note = Nucleic acid sequence of hz39D2.23 heavy chain
  source 1..1344: mol_type = other DNA

SEQ ID NO: 107 - protein, 107 aa, synthetic construct
  REGION 1..107: note = Amino acid sequence of hz39D2.23 light chain variable region
DIQMTQSPSSLSASVGDRVTITCKASQDINSYLSWFQQKPGKAPKTLIYRANRLVDGVPSRFSGSGSGQDYTLTISSLQPEDFATYYCLQLDEFPWTFGQGTKVEIK

SEQ ID NO: 108 - DNA, 321 bp, synthetic construct
  misc_feature 1..321: note = Nucleic acid sequence of hz39D2.23 light chain variable region
  source 1..321: mol_type = other DNA
gacattcaaatgacacagtctcccagctcccttagtgcttcggtgggcgatcgggtgaccataacatgcaaggcctcacaggacatcaacagctatctctcatggtttcagcagaagccaggaaaagcacctaaaacgttgatctacagggccaatcgcctcgttgacggagtcccctccagattcagcgggagtgggtctggtcaggattatactctgaccatctcctctctgcagcctgaagactttgccacttactactgtctgcaactcgatgagttcccatggaccttcggccagggcaccaaggtggagattaaa

SEQ ID NO: 109 - protein, 214 aa, synthetic construct
  REGION 1..214: note = Amino acid sequence of hz39D2.23 light chain
DIQMTQSPSSLSASVGDRVTITCKASQDINSYLSWFQQKPGKAPKTLIYRANRLVDGVPSRFSGSGSGQDYTLTISSLQPEDFATYYCLQLDEFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - DNA, 642 bp, synthetic construct
  misc_feature 1..642: note = Nucleic acid sequence of hz39D2.23 light chain
  source 1..642: mol_type = other DNA
gacattcaaatgacacagtctcccagctcccttagtgcttcggtgggcgatcgggtgaccataacatgcaaggcctcacaggacatcaacagctatctctcatggtttcagcagaagccaggaaaagcacctaaaacgttgatctacagggccaatcgcctcgttgacggagtcccctccagattcagcgggagtgggtctggtcaggattatactctgaccatctcctctctgcagcctgaagactttgccacttactactgtctgcaactcgatgagttcccatggaccttcggccagggcaccaaggtggagattaaacggaccgtggccgctccctccgtgttcatcttcccaccctccgacgagcagctgaagtccggcaccgccagcgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 111 - protein, 21 aa, synthetic construct
  REGION 1..21: note = Signal peptide (CD8alpha)
MALPVTALLLPLALLLHAARP

SEQ ID NO: 112 - DNA, 63 bp, synthetic construct
  misc_feature 1..63: note = Signal peptide (CD8alpha)
  source 1..63: mol_type = other DNA
atggctctgccagtgactgcactgctgctgccactggccctgctgctgcacgcagctcgacct

SEQ ID NO: 113 - protein, 240 aa, synthetic construct
  REGION 1..240: note = Anti-HER2 scFv
DIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSS

SEQ ID NO: 114 - DNA, 720 bp, synthetic construct
  misc_feature 1..720: note = Anti-HER2 scFv
  source 1..720: mol_type = other DNA
gacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagc

SEQ ID NO: 115 - protein, 48 aa, synthetic construct
  REGION 1..48: note = Hinge (CD8alpha)
AKPTTTPAPRPPTPAPTIASQPLSLRPEACRPAAGGAVHTRGLDFACD

SEQ ID NO: 116 - DNA, 144 bp, synthetic construct
  misc_feature 1..144: note = Hinge (CD8alpha)
  source 1..144: mol_type = other DNA
gcaaaacctaccacaactcctgcaccacgcccccctactccagcacctaccatcgcatctcagccactgagtctgcgaccagaggcctgccggcccgccgccggcggggccgtccataccagagggctggactttgcctgcgat

SEQ ID NO: 117 - protein, 24 aa, synthetic construct
  REGION 1..24: note = Transmembrane (CD8alpha)
IYIWAPLAGTCGVLLLSLVITLYC

SEQ ID NO: 118 - DNA, 72 bp, synthetic construct
  misc_feature 1..72: note = Transmembrane (CD8alpha)
  source 1..72: mol_type = other DNA
atctacatttgggcccctctggctggaacatgtggcgtgctgctgctgtccctggtcattactctgtattgt

SEQ ID NO: 119 - protein, 27 aa, synthetic construct
  REGION 1..27: note = Transmembrane (CD28)
FWVLVVVGGVLACYSLLVTVAFIIFWV

SEQ ID NO: 120 - DNA, 81 bp, synthetic construct
  misc_feature 1..81: note = Transmembrane (CD28)
  source 1..81: mol_type = other DNA
ttttgggtcctggtggtcgtgggaggggtgctggcatgttactcactgctggtcaccgtggccttcatcatcttctgggtg

SEQ ID NO: 121 - protein, 112 aa, synthetic construct
  REGION 1..112: note = Intracellular stimulatory Signal- (CD3-lambda)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - DNA, 339 bp, synthetic construct
  misc_feature 1..339: note = Intracellular stimulatory Signal- (CD3-lambda)
  source 1..339: mol_type = other DNA
cgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 123 - protein, 42 aa, synthetic construct
  REGION 1..42: note = Intracellular stimulatory signal (4-1BB)
KRGRKKLLYIFKQPFMRPVQTTQEEDGCSCRFPEEEEGGCEL

SEQ ID NO: 124 - DNA, 126 bp, synthetic construct
  misc_feature 1..126: note = Intracellular stimulatory signal (4-1BB)
  source 1..126: mol_type = other DNA
aagcggggaagaaagaaactgctgtacatcttcaaacagccctttatgaggcctgtgcagaccacacaggaggaagacggctgctcctgccggttccccgaggaagaggaaggcgggtgcgagctg

SEQ ID NO: 125 - protein, 41 aa, synthetic construct
  REGION 1..41: note = Intracellular stimulatory signal (CD28)
RSKRSRLLHSDYMNMTPRRPGPTRKHYQPYAPPRDFAAYRS

SEQ ID NO: 126 - DNA, 123 bp, synthetic construct
  misc_feature 1..123: note = Intracellular stimulatory signal (CD28)
  source 1..123: mol_type = other DNA
cggagcaagaggtcccgcctgctgcacagcgactatatgaacatgaccccacggagacccggccctacacggaaacattaccagccctatgctccaccccgggacttcgcagcttacagaagt

SEQ ID NO: 127 - protein, 22 aa, synthetic construct
  REGION 1..22: note = Intracellular stimulatory signal (OX40 ligand)
ERVQPLEENVGNAARPRFERNK

SEQ ID NO: 128 - DNA, 66 bp, synthetic construct
  misc_feature 1..66: note = Intracellular stimulatory signal (OX40 ligand)
  source 1..66: mol_type = other DNA
gaaagagtgcagcccctggaagagaatgtcgggaatgccgctcgcccaagatttgaaaggaacaaa

SEQ ID NO: 129 - protein, 445 aa, synthetic construct
  REGION 1..445: note = Her2-Z CAR (Clone #2)
MALPVTALLLPLALLLHAARPDIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - DNA, 1338 bp, synthetic construct
  misc_feature 1..1338: note = Her2-Z CAR (Clone #2)
  source 1..1338: mol_type = other DNA
atggctctgccagtgactgcactgctgctgccactggccctgctgctgcacgcagctcgacctgacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagcgcaaaacctaccacaactcctgcaccacgcccccctactccagcacctaccatcgcatctcagccactgagtctgcgaccagaggcctgccggcccgccgccggcggggccgtccataccagagggctggactttgcctgcgatatctacatttgggcccctctggctggaacatgtggcgtgctgctgctgtccctggtcattactctgtattgtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 131 - protein, 487 aa, synthetic construct
  REGION 1..487: note = Her2-BBZ CAR (Clone #3)
MALPVTALLLPLALLLHAARPDIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - DNA, 1464 bp, synthetic construct
  misc_feature 1..1464: note = Her2-BBZ CAR (Clone #3)
  source 1..1464: mol_type = other DNA
atggctctgccagtgactgcactgctgctgccactggccctgctgctgcacgcagctcgacctgacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagcgcaaaacctaccacaactcctgcaccacgcccccctactccagcacctaccatcgcatctcagccactgagtctgcgaccagaggcctgccggcccgccgccggcggggccgtccataccagagggctggactttgcctgcgatatctacatttgggcccctctggctggaacatgtggcgtgctgctgctgtccctggtcattactctgtattgtaagcggggaagaaagaaactgctgtacatcttcaaacagccctttatgaggcctgtgcagaccacacaggaggaagacggctgctcctgccggttccccgaggaagaggaaggcgggtgcgagctgcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 133 - protein, 489 aa, synthetic construct
  REGION 1..489: note = Her2-28Z CAR (Clone #6)
MALPVTALLLPLALLLHAARPDIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - DNA, 1470 bp, synthetic construct
  misc_feature 1..1470: note = Her2-28Z CAR (Clone #6)
  source 1..1470: mol_type = other DNA
atggctctgccagtgactgcactgctgctgccactggccctgctgctgcacgcagctcgacctgacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagcgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 135 - protein, 511 aa, synthetic construct
  REGION 1..511: note = Her2-28OX40LZ CAR (Clone #14)
MALPVTALLLPLALLLHAARPDIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - DNA, 1536 bp, synthetic construct
  misc_feature 1..1536: note = Her2-28OX40LZ CAR (Clone #14)
  source 1..1536: mol_type = other DNA
atggctctgccagtgactgcactgctgctgccactggccctgctgctgcacgcagctcgacctgacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagcgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtgaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

(19) United States
(12) Patent Application Publication
(10) Pub. No.: US 2024/0270872 A1
(43) Pub. Date: Aug. 15, 2024

(54) ANTI-HER2 ANTIBODY OR ANTIGEN-BINDING FRAGMENT THEREOF, AND CHIMERIC ANTIGEN RECEPTOR COMPRISING SAME

(71) Applicant: GC Cell Corporation, Yongin-si (KR)

(72) Inventors: Jong Seo Lee, Yongin-si (KR); Kyu Tae Kim, Yongin-si (KR); Young Ha Lee, Yongin-si (KR); In Sik Hwang, Yongin-si (KR); Bong Kook Ko, Yongin-si (KR); Eunji Choi, Yongin-si (KR); You-Sun Kim, Yongin-si (KR); Jeongmin Kim, Yongin-si (KR); Miyoung Jung, Yongin-si (KR); Hoyong Lim, Yongin-si (KR); Sungyoo Cho, Yongin-si (KR)

(21) Appl. No.: 18/618,414

(22) Filed: Mar. 27, 2024

Related U.S. Application Data
(63) Continuation of application No. 18/298,310, filed on Apr. 10, 2023, now Pat. No. 11,970,547.
Application No. 18/298,310 is a division of application No. 16/881,650, filed on May 22, 2020, now Pat. No. 11,649,294.
Application No. 16/881,650 is a continuation-in-part of application No. 16/764,276, filed on Sep. 28, 2020.

(30) Foreign Application Priority Data
Nov. 14, 2017 (KR) 1020170151841

(51) Int. Cl. C07K 16/32; A61K 38/17; A61P 35/00; C07K 14/705; C12N 5/0783

(52) U.S. Cl. CPC C07K 16/32; A61K 38/177; A61P 35/00; C07K 14/705; C12N 5/0646; C07K 2317/565

(57) ABSTRACT

The present disclosure relates to a novel anti-HER2 antibody or an antigen-binding fragment thereof used in the prevention or treatment of cancer, a chimeric antigen receptor including the same, and uses thereof. The antibody of the present disclosure is an antibody that specifically binds to HER2 which is highly expressed in cancer cells (particularly, breast cancer or gastric cancer cells), and binds to an epitope that is different from an epitope to which trastuzumab binds.

### CROSS-REFERENCE TO RELATED APPLICATIONS

This application is a continuation application of and claims the benefit of priority to U.S. application Ser. No. 18/298,310, filed on Apr. 10, 2023, which is a divisional application of and claims the benefit of priority to U.S. application Ser. No. 16/881,650, filed on May 22, 2020, now U.S. Pat. No. 11,649,294, which is a continuation-in-part application of and claims the benefit of priority to U.S. application Ser. No. 16/764,276, filed on Sep. 28, 2020, which is a National Stage of International Application No. PCT/KR2018/013928, filed on Nov. 14, 2018, claiming priority based on Korean Patent Application No. 10-2017-0151841, filed Nov. 14, 2017, the contents of which are hereby incorporated by reference.

### REFERENCE TO SEQUENCE LISTING SUBMITTED ELECTRONICALLY

This application contains a Sequence Listing that has been submitted electronically as an XML file named 49755-0034003_SL_ST26.xml. The XML file, created on Mar. 27, 2024, is 214,314 bytes in size. The material in the XML file is hereby incorporated by reference in its entirety.

### TECHNICAL FIELD

The research was conducted under the support of the Ministry of Trade, Industry and Energy of Korea with the project number 1415118385. The R&D management agency of the project is the Korea Institute for Advancement of Technology, the R&D project title is “Global innovation technology alliance”, and the research title is “Development of global antibody drug based on novel epitope screening platform technology”. The research was conducted by AbClon Inc. from Nov. 1, 2011 until Oct. 31, 2014.

This application claims the priority of Korean Patent Application No. 10-2017-0151841 filed on Nov. 14, 2017 in the Korean Intellectual Property Office, the disclosure of which is incorporated herein by reference in its entirety.

The present disclosure relates to a novel anti-HER2 antibody or an antigen-binding fragment thereof, a chimeric antigen receptor including the same, and uses thereof.

### BACKGROUND ART

The Her2/neu (ErbB2) gene encodes a 185-kDa transmembrane glycoprotein that belongs to the family of epidermal growth factor receptors (EGFRs). The Her2 protein is composed of an extracellular domain consisting of 620 amino acid residues, a transmembrane domain 23 amino acid residues, and an intracellular domain with tyrosine kinase activity, consisting of 490 amino acid residues (Akiyama T, et al., Science, 232 (4758): 1644-1646 (1986)).

Anti-HER2 antibodies with various characteristics have been described: Tagliabue et al., Int. J. Cancer 47: 933-937 (1991); McKenzie et al., Oncogene 4: 543-548 (1989); Maier et al., Cancer Res. 51: 5361-5369 (1991); Bacus et al., Molecular Carcinogenesis 3: 350-362 (1990); Stancovski et al., PNAS USA 88: 8691-8695 (1991); Bacus et al., Cancer Research 52: 2580-2589 (1992); Xu et al., Int. J. Cancer 53: 401-408 (1993); WO94/00136; Kasprzyk et al., Cancer Research 52: 2771-2776 (1992); Hancock et al., Cancer Res. 51: 4575-4580 (1991); Shawver et al., Cancer Res. 54: 1367-1373 (1994); Arteaga et al., Cancer Res. 54: 3758-3765 (1994); Harwerth et al., J. Biol. Chem. 267: 15160-15167 (1992); U.S. Pat. No. 5,783,186; Kao et al., US Patent Application Publication No. 2009/0285837 (2009); Ross et al., The Oncologist 8: 307-325 (2003); and Klapper et al., Oncogene 14: 2099-2109 (1997).

The most commercially successful anti-HER2 antibody is trastuzumab antibody (commercially available as Herceptin™, U.S. Pat. No. 5,821,337) and many researches have been conducted thereon: Sapino, A., et al., Annals of Oncology (2007) 18: 1963-1968; Bussolati, G, et al., British Journal of Cancer (2005) 92, 1261-1267; and Glazyrin A, et al., J Histology & Cytochemistry (2007) 55 (1): 25-33.

Although the trastuzumab antibody has been commercially successful, use of the trastuzumab antibody for therapeutic purposes is limited because there are various cancer cells which have non-reactivity (or resistance) to the antibody or have reduced sensitivity. Accordingly, there have been attempts to resolve the therapeutic problem of the antibody.

For example, U.S. Pat. No. 7,674,460 discloses a method for increasing the HER2 sensitivity of cancer cells using an HER2 antagonist such as the trastuzumab antibody and a PC cell-derived growth factor (PCDGF) antagonist. WO 2011/127297 discloses a method for inhibiting the proliferation of trastuzumab-resistant tumor cells using a combination of a FoxMl inhibitor and the trastuzumab antibody.

US Patent Application Publication No. 2010-0183604 discloses a method for treating trastuzumab-resistant cancer using a cofilin inhibitor, a PAK1 inhibitor, a LIMK inhibitor, an RHO inhibitor, a ROCK1 inhibitor or a ROCK2 inhibitor.

### DISCLOSURE

### Technical Problem

The inventors of the present disclosure have made efforts to develop a novel antibody which is capable of preventing or treating cancer (particularly, breast cancer and gastric cancer), exhibits better killing ability (or proliferation-inhibiting ability) for cancer cells which have non-reactivity (or resistance) to the trastuzumab antibody or have reduced sensitivity, and is capable of preventing or treating cancer with improved anticancer activity when co-administered with the trastuzumab antibody as compared to single administration of trastuzumab. As a result, they have developed a novel antibody which exhibits better killing ability for HER2-overexpressed cancer cells on which the trastuzumab antibody hardly acts, or exhibits improved anticancer activity when co-administered with the trastuzumab antibody, and have completed the present disclosure.

### SUMMARY OF THE INVENTION

The present disclosure provides an antibody or an antigen binding fragment thereof against HER2 (human epidermal growth factor receptor 2) comprising any one of: (a) a heavy chain variable region comprising a CDRH1 of SEQ ID NO 1, a CDRH2 of SEQ ID NO 2 and a CDRH3 of SEQ ID NO 3, and a light chain variable region comprising a CDRL1 of SEQ ID NO 4, a CDRL2 of SEQ ID NO 5 and a CDRL3 of SEQ ID NO 6; (b) a heavy chain variable region comprising a CDRH1 of SEQ ID NO 7, a CDRH2 of SEQ ID NO 8 and a CDRH3 of SEQ ID NO 9, 71 or 72, and a light chain variable region comprising a CDRL1 of SEQ ID NO 10, a CDRL2 of SEQ ID NO 11 and a CDRL3 of SEQ ID NO 12, 73 or 74; (c) a heavy chain variable region comprising a CDRH1 of SEQ ID NO 13, a CDRH2 of SEQ ID NO 14 and a CDRH3 of SEQ ID NO 15, and a light chain variable region comprising a CDRL1 of SEQ ID NO 16, a CDRL2 of SEQ ID NO 17 and a CDRL3 of SEQ ID NO 18; (d) a heavy chain variable region comprising a CDRH1 of SEQ ID NO 19, a CDRH2 of SEQ ID NO 20 and a CDRH3 of SEQ ID NO 21, and a light chain variable region comprising a CDRL1 of SEQ ID NO 22, a CDRL2 of SEQ ID NO 23 and a CDRL3 of SEQ ID NO 24; or (d) a heavy chain variable region comprising a CDRH1 of SEQ ID NO 25, a CDRH2 of SEQ ID NO 26 and a CDRH3 of SEQ ID NO 27, and a light chain variable region comprising a CDRL1 of SEQ ID NO 28, a CDRL2 of SEQ ID NO 29 and a CDRL3 of SEQ ID NO 30.

In some aspects of the disclosure, the heavy chain variable region of (a) comprises an amino acid sequence of SEQ ID NO 31 or 75; the heavy chain variable region of (b) comprises an amino acid sequence of SEQ ID NO 39, 83, 87, 95 or 103; the heavy chain variable region of (c) comprises an amino acid sequence of SEQ ID NO 47; the heavy chain variable region of (d) comprises an amino acid sequence of SEQ ID NO 55; and the heavy chain variable region of (e) comprises an amino acid sequence of SEQ ID NO 63 or 79.

In some aspects of the disclosure, the light chain variable region of (a) comprises an amino acid sequence of SEQ ID NO 35 or 77; the light chain variable region of (b) comprises an amino acid sequence of SEQ ID NO 43, 85, 91, 99 or 107; the light chain variable region of (c) comprises an amino acid sequence of SEQ ID NO 51; the light chain variable region of (d) comprises an amino acid sequence of SEQ ID NO 59; and the light chain variable region of (e) comprises an amino acid sequence of SEQ ID NO 67 or 81.

In some aspects of the disclosure, the antibody or the antigen-binding fragment thereof comprising (a) comprises a heavy chain comprising an amino acid sequence of SEQ ID NO 33; the antibody or the antigen-binding fragment thereof comprising (b) comprises a heavy chain comprising an amino acid sequence of SEQ ID NO 41, 89, 97 or 105; the antibody or the antigen-binding fragment thereof comprising (c) comprises a heavy chain comprising an amino acid sequence of SEQ ID NO 49; the antibody or the antigen-binding fragment thereof comprising (d) comprises a heavy chain comprising an amino acid sequence of SEQ ID NO 57; and the antibody or the antigen-binding fragment thereof comprising (e) comprises a heavy chain comprising an amino acid sequence of SEQ ID NO 65.

In some aspects of the disclosure, the antibody or the antigen-binding fragment thereof comprising (a) comprises a light chain comprising an amino acid sequence of SEQ ID NO 37; the antibody or the antigen-binding fragment thereof comprising (b) comprises a light chain comprising an amino acid sequence of SEQ ID NO 45, 93, 101 or 109; the antibody or the antigen-binding fragment thereof comprising (c) comprises a light chain comprising an amino acid sequence of SEQ ID NO 53; the antibody or the antigen-binding fragment thereof comprising (d) comprises a light chain comprising an amino acid sequence of SEQ ID NO 61; and the antibody or the antigen-binding fragment thereof comprising (e) comprises a light chain comprising an amino acid sequence of SEQ ID NO 69.

The present disclosure further provides a fusion protein comprising the antibody or the antigen-binding fragment as described herein.

The present disclosure further provides a chimeric antigen receptor polypeptide comprising an HER2-binding domain; a transmembrane domain (TM); a costimulatory domain; and an intracellular signaling domain (ICD).

In some aspects of the disclosure, the HER2-binding domain comprises the antibody or the antigen-binding fragment thereof as described herein.

In some aspects of the disclosure, the transmembrane domain is a transmembrane domain of a protein selected from a group consisting of T-cell receptor alpha, beta or zeta chain, CD28, CD3 epsilon, CD45, CD4, CD5, CD8, CD9, CD16, CD22, CD33, CD37, CD64, CD80, CD86, CD134, CD137 and CD154.

In some aspects of the disclosure, the costimulatory domain is a functional signaling domain obtained from a protein selected from a group consisting of MHC class I molecule, TNF receptor protein, immunoglobulin-like protein, cytokine receptor, integrin, signaling lymphocytic activation molecule (SLAM), activating NK cell receptor, BTLA (B- and T-lymphocyte attenuator), Toll-like ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, and a ligand binding specifically to CD83.

In some aspects of the disclosure, the intracellular signaling domain comprises a functional signaling domain of 4-1BB, CD28, OX40 or CD3 zeta, or a combination thereof.

In some aspects of the disclosure, the intracellular signaling domain comprises a functional signaling domain of OX40 ligand.

The present disclosure further provides a nucleic acid molecule encoding the anti-HER2 antibody or the antigen-binding fragment thereof as described herein.

The present disclosure further provides a nucleic acid molecule encoding the chimeric antigen receptor polypeptide as described herein.

The present disclosure further provides a recombinant vector comprising the nucleic acid molecule encoding the anti-HER2 antibody or the antigen-binding fragment thereof as described herein or the chimeric antigen receptor polypeptide as described herein.

The present disclosure further provides a host cell transformed with the recombinant vector as described herein.

The present disclosure further provides an effector cell expressing the chimeric antigen receptor polypeptide as described herein.

In some aspects of the disclosure, the effector cell is selected from a group consisting of a dendritic cell, a killer dendritic cell, a mast cell, a natural killer cell, a B lymphocyte, a T lymphocyte, a macrophage and precursor cells thereof.

In some aspects of the disclosure, the T lymphocyte is selected from a group consisting of an inflammatory T lymphocyte, a cytotoxic T lymphocyte, a regulatory T lymphocyte or a helper T lymphocyte.

The present disclosure further provides a pharmaceutical composition for preventing or treating cancer, comprising a pharmaceutically effective amount of the anti-HER2 antibody or the antigen-binding fragment thereof as described herein a pharmaceutically acceptable carrier.

The present disclosure further provides a pharmaceutical composition for treating cancer, comprising the effector cell expressing the chimeric antigen receptor polypeptide as described herein.

In some aspects of the disclosure, the cancer is breast cancer, ovarian cancer, gastric cancer, lung cancer, liver cancer, bronchial cancer, nasopharyngeal cancer, laryngeal cancer, pancreatic cancer, bladder cancer, colorectal cancer, colon cancer, cervical cancer, brain cancer, prostate cancer, bone cancer, head and neck cancer, skin cancer, thyroid cancer, parathyroid cancer or ureteral cancer.

In some aspects of the disclosure, the pharmaceutical composition further comprises the trastuzumab antibody.

The present disclosure further provides a kit for diagnosing cancer, comprising the anti-HER2 antibody or the antigen-binding fragment thereof as described herein.

The present disclosure further provides a chimeric antigen receptor comprising an extracellular domain that binds Her2, wherein the extracellular domain comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 113 (hz39D2 (VL-GS linker-VH)).

In some aspects of the disclosure, the chimeric antigen receptor further comprises an extracellular signaling domain linked to the extracellular domain; a hinge domain linked to the extracellular domain; a transmembrane domain linked to the hinge domain; and
an intracellular stimulatory signal linked to the hinge domain.

In some aspects of the disclosure, the extracellular signaling domain comprises an amino acid sequence having at least 80%, at least 85%, at least 90% at least 91%, at least 92%, at least 93%, at least 94%, at least 95%, at least 96%, at least 97%, at least 98%, or at least 99% sequence identity with SEQ ID NO: 111 (CD8a signal peptide). In some aspects, the extracellular signaling domain comprises SEQ ID NO: 111 (CD8a signal peptide).

In some aspects of the disclosure, the hinge domain comprises an amino acid sequence having at least 80%, at least 85%, at least 90%, at least 91%, at least 92%, at least 93%, at least 94%, at least 95%, at least 96%, at least 97%, at least 98%, or at least 99% sequence identity with SEQ ID NO: 115 (CD8α hinge). In some aspects of the disclosure, the hinge domain comprises SEQ ID NO: 115 (CD8α hinge).

In some aspects of the disclosure, the transmembrane domain comprises an amino acid sequence having at least 80%, at least 85%, at least 90%, at least 91%, at least 92%, at least 93%, at least 94%, at least 95%, at least 96%, at least 97%, at least 98%, or at least 99% sequence identity with SEQ ID NO: 117 (CD8α TM) or SEQ ID NO: 119 (CD28 TM). In some aspects of the disclosure, the transmembrane domain comprises SEQ ID NO: 117 CD8α TM) or SEQ ID NO: 119 (CD28 TM).

In some aspects of the disclosure, the intracellular stimulatory signal comprises an amino acid sequence having at least 80%, at least 85%, at least 90%, at least 91%, at least 92%, at least 93%, at least 94%, at least 95%, at least 96%, at least 97%, at least 98%, or at least 99% sequence identity with SEQ ID NO: 121 (CD3-ζ). In some aspects, the intracellular stimulatory signal comprises SEQ ID NO: 121 (CD3-ζ).

In some aspects of the disclosure, the chimeric antigen receptor further comprises a second intracellular stimulatory signal, wherein the second intracellular stimulatory signal comprises an amino acid sequence having at least 80%, at least 85%, at least 90%, at least 91%, at least 92%, at least 93%, at least 94%, at least 95%, at least 96%, at least 97%, at least 98%, or at least 99% sequence identity with SEQ ID NO: 123 (4-1BB) or SEQ ID NO: 125 (CD28). In some aspects of the disclosure, the second intracellular stimulatory signal comprises SEQ ID NO: 123 (4-1BB) or SEQ ID NO: 125 (CD28).

In some aspects of the disclosure, the chimeric antigen receptor further comprises a third intracellular stimulatory signal, wherein the third intracellular stimulatory signal comprises an amino acid sequence having at least 80%, at least 85%, at least 90%, at least 91%, at least 92%, at least 93%, at least 94%, at least 95%, at least 96%, at least 97%, at least 98%, or at least 99% sequence identity with SEQ ID NO: 127 (OX40L). In some aspects of the disclosure, wherein the third intracellular stimulatory signal comprises SEQ ID NO: 127 (OX40L).

In some aspects of the disclosure, the chimeric antigen receptor comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 129 (Clone 2), SEQ ID NO: 131 (Clone 3), SEQ ID NO: 133 (Clone 6), or SEQ ID NO: 135 (Clone 14). In some aspects of the disclosure, the chimeric antigen receptor comprises SEQ ID NO: 129 (Clone 2), SEQ ID NO: 131 (Clone 3), SEQ ID NO: 133 (Clone 6), or SEQ ID NO: 135 (Clone 14).

The present disclosure further provides a nucleic acid molecule encoding the chimeric antigen receptor having an extracellular domain comprising an amino acid sequence with at least 80%, at least 85%, at least 90%, at least 91%, at least 92%, at least 93%, at least 94%, at least 95%, at least 96%, at least 97%, at least 98%, or at least 99% sequence identity to SEQ ID NO: 113 (hz39D2 (VL-GS linker-VH)) as described herein.

The present disclosure further provides a vector comprising the nucleic acid molecule encoding the chimeric antigen receptor having an extracellular domain comprising an amino acid sequence with at least 80%, at least 85%, at least 90%, at least 91%, at least 92%, at least 93%, at least 94%, at least 95%, at least 96%, at least 97%, at least 98%, or at least 99% sequence identity to SEQ ID NO: 113 (hz39D2 (VL-GS linker-VH)) as described herein.

The present disclosure further provides an immune cell expressing the chimeric antigen having an extracellular domain comprising an amino acid sequence with at least 80%, at least 85%, at least 90%, at least 91%, at least 92%, at least 93%, at least 94%, at least 95%, at least 96%, at least 97%, at least 98%, or at least 99% sequence identity to SEQ ID NO: 113 (hz39D2 (VL-GS linker-VH)) as described herein.

In some aspects of the disclosure, the immune cell is a natural killer cell.

The present disclosure further provides a pharmaceutical composition comprising the immune cell as described herein and a pharmaceutically acceptable carrier.

The present disclosure further provides a method for treating cancer comprising administering to a subject in need thereof a therapeutically effective amount of the pharmaceutical composition as described herein.

In some aspects of the disclosure, the cancer is selected from the group consisting of breast cancer, ovarian cancer, gastric cancer, lung cancer, liver cancer, bronchial cancer, nasopharyngeal cancer, laryngeal cancer, pancreatic cancer, bladder cancer, colorectal cancer, colon cancer, cervical cancer, brain cancer, prostate cancer, bone cancer, head and neck cancer, skin cancer, thyroid cancer, parathyroid cancer and ureteral cancer.

### BRIEF DESCRIPTION OF DRAWINGS

FIG. 1 is a line graph showing the a results of analyzing the affinity of hz2G10, hz39D2, 24D3, 1G3 and hz8G11 clones for the HER2-ECD-Fc antigen by ELISA.

FIG. 2 is a bar graph showing the a results of investigating the extracellular domain of HER2 to which hz2G10, hz39D2, 24D3, 1G3 and hz8G11 clones bind.

FIG. 3A and FIG. 3B are line graphs showing the results of analyzing the effect of single administration of five antibodies of the present disclosure (hz2G10, hz39D2, 24D3, 1G3 and hz8G11) on the inhibition of the growth of HER2-overexpressed breast cancer cells (SKBR3) and HER2-unexpressed breast cancer cells (MCF-7).

FIG. 3C and FIG. 3D are line graphs showing the results of analyzing the effect of co-administration of five antibodies of the present disclosure (hz2G10, hz39D2, 24D3, 1G3 and hz8G11) and the trastuzumab (TRA) antibody on the inhibition of the growth of HER2-overexpressed breast cancer cells (SKBR3) and HER2-unexpressed breast cancer cells (MCF-7).

FIG. 4 is a bar graph showing the results of investigating the specificity of antibodies developed by expressing the ErbB family. Cetuximab (CET), trastuzumab (TRA) and patritumab (AMG888, AMG) were used as control groups binding to EGFR, HER2 and HER3, respectively.

FIGS. 5A-5E are line graphs showing the results of comparing the epitopes of developed antibodies with trastuzumab. For comparison with the epitope of trastuzumab, trastuzumab and HER2-His were immobilized on a sensor chip and then the binding with five antibodies of the present disclosure was analyzed.

FIGS. 6A-6C are bar graphs showing the results of analyzing the effect of single administration of hz39D2 and affinity-improved clones thereof (hz39D2.14, hz39D2.22 and hz39D2.23) or co-administration with the trastuzumab antibody on the inhibition of the growth of HER2-overexpressed gastric cancer and breast cancer cells.

FIG. 7 is a table providing a summary of HER2-CAR constructs according to the present disclosure.

FIGS. 8A-8C are line graphs showing the results of a cell killing assay (Calcein releasing cytotoxicity assay) assessing the cytotoxicity of cord-blood derived NK cells (CBNKs) expressing HER2-CAR constructs against HER2 positive target cancer cell lines.

FIGS. 9A-9E are line graphs showing the results of a cell killing assay (Calcein releasing cytotoxicity assay) assessing the cytotoxicity of cord-blood derived NK cells expressing HER2-CAR construct clone #14 against HER2 positive target cancer cell lines.

FIGS. 10A-10B are line graphs showing the long-term serial killing activity of HER2-CAR clones #6 and #14 assessed using the Incucyte live cell imaging system.

FIGS. 11A-11B are bar graphs showing the NK cell degranulation activity and cytotoxic cytokine expression with the various HER2-CAR constructs evaluated by comparing intercellular expression level of CD107a and IFN-7.

FIGS. 12A-12C are bar graphs showing NK cell degranulation activity and cytotoxic cytokine expression of HER2-CAR clones #6 and #14 evaluated by comparing intercellular expression level of CD107a, IFN-γ, and TNF-α.

FIG. 13 is a bar graph showing the secretion of IFN-γ from anti-HER2-CAR-CBNKs (clones #2, #6, #14) when co-cultured with target cells.

FIGS. 14A-14B are a scheme and line graph, respectively, showing the in vivo efficacy of anti-Her2-CAR clones #3 and #14 Xenograft models in NSG mice were generated by injecting 5×10⁶ HCC1954 cells/mouse (HER-2 positive, trastuzumab resistant cells) subcutaneously. Tumor volume was assessed every 3-4 days after injection.

FIG. 15A is a line graph showing the proliferation of HER2-CAR-NK cells. FIGS. 15B-15D are line graphs (FIG. 15B) and bar graphs (FIGS. 15C and 15D) showing the CAR expression levels, viability, and purity of HER2-CAR-NK cells during expansion, culture and cryopreservation.

FIG. 16 is a bar graph showing the phenotypic cell surface marker expression of HER2-CAR-NK cells.

### DETAILED DESCRIPTION OF THE INVENTION

The present disclosure is directed to providing an antibody (anti-HER2 antibody) against HER2 (human epidermal growth factor receptor 2) or an antigen-binding fragment thereof.

The present disclosure is also directed to providing a fusion protein including the anti-HER2 antibody or an antigen-binding fragment thereof.

The present disclosure is also directed to providing a chimeric antigen receptor (CAR) including the anti-HER2 antibody or an antigen-binding fragment thereof and an effector cell expressing the same.

The present disclosure is also directed to providing a nucleic acid molecule encoding the anti-HER2 antibody or an antigen-binding fragment thereof; or the chimeric antigen receptor.

The present disclosure is also directed to providing a recombinant vector including the nucleic acid molecule.

The present disclosure is also directed to providing a host cell transformed with the recombinant vector.

The present disclosure is also directed to providing a pharmaceutical composition for preventing or treating cancer, which contains the anti-HER2 antibody or an antigen-binding fragment thereof.

The present disclosure is also directed to providing a kit for diagnosing cancer, which includes the anti-HER2 antibody or an antigen-binding fragment thereof.

The present disclosure is also directed to providing a method for preventing or treating cancer by administering a composition containing the anti-HER2 antibody or an antigen-binding fragment thereof to a subject.

The present disclosure is also directed to providing a method for treating a disease related with HER2 overexpression (e.g., cancer) by administering an effector cell expressing the chimeric antigen receptor to a subject.

### Technical Solution

The present disclosure provides an antibody binding specifically to HER2 (human epidermal growth factor receptor 2) and modified antibodies thereof that have undergone affinity maturation.

A first aspect of the present disclosure provides an antibody against HER2 (human epidermal growth factor receptor 2) including the followings or an antigen-binding fragment thereof:
(a) a heavy chain variable region including the following heavy chain CDR (complementarity-determining region) amino acid sequences:
   CDRH1 of SEQ ID NO 1, CDRH2 of SEQ ID NO 2 and CDRH3 of SEQ ID NO 3; and
(b) a light chain variable region including the following light chain CDR amino acid sequences:
   CDRL1 of SEQ ID NO 4, CDRL2 of SEQ ID NO 5 and CDRL3 of SEQ ID NO 6.

A second aspect of the present disclosure provides an antibody against HER2 (human epidermal growth factor receptor 2) including the followings or an antigen-binding fragment thereof:
(a) a heavy chain variable region including the following heavy chain CDR (complementarity-determining region) amino acid sequences:
   CDRH1 of SEQ ID NO 7, CDRH2 of SEQ ID NO 8 and CDRH3 of SEQ ID NO 9, 71 or 72; and
(b) a light chain variable region including the following light chain CDR amino acid sequences:
   CDRL1 of SEQ ID NO 10, CDRL2 of SEQ ID NO 11 and CDRL3 of SEQ ID NO 12, 73 or 74.

A third aspect of the present disclosure provides an antibody against HER2 (human epidermal growth factor receptor 2) including the following or an antigen-binding fragment thereof:
(a) a heavy chain variable region including the following heavy chain CDR (complementarity-determining region) amino acid sequences:
   CDRH1 of SEQ ID NO 13, CDRH2 of SEQ ID NO 14 and CDRH3 of SEQ ID NO 15; and
(b) a light chain variable region including the following light chain CDR amino acid sequences:
   CDRL1 of SEQ ID NO 16, CDRL2 of SEQ ID NO 17 and CDRL3 of SEQ ID NO 18.

A fourth aspect of the present disclosure provides an antibody against HER2 (human epidermal growth factor receptor 2) including the following or an antigen-binding fragment thereof:
(a) a heavy chain variable region including the following heavy chain CDR (complementarity-determining region) amino acid sequences:
   CDRH1 of SEQ ID NO 19, CDRH2 of SEQ ID NO 20 and CDRH3 of SEQ ID NO 21; and
(b) a light chain variable region including the following light chain CDR amino acid sequences:
   CDRL1 of SEQ ID NO 22, CDRL2 of SEQ ID NO 23 and CDRL3 of SEQ ID NO 24.

A fifth aspect of the present disclosure provides an antibody against HER2 (human epidermal growth factor receptor 2) including the following or an antigen-binding fragment thereof:
(a) a heavy chain variable region including the following heavy chain CDR (complementarity-determining region) amino acid sequences:
   CDRH1 of SEQ ID NO 25, CDRH2 of SEQ ID NO 26 and CDRH3 of SEQ ID NO 27; and
(b) a light chain variable region including the following light chain CDR amino acid sequences:
   CDRL1 of SEQ ID NO 28, CDRL2 of SEQ ID NO 29 and CDRL3 of SEQ ID NO 30.

The antibody of the first aspect, the antibody of the second aspect, the antibody of the third aspect, the antibody of the fourth aspect and the antibody of the fifth aspect are referred to, respectively, as 2G10, 39D2, 24D3, 1G3 and 8G11 antibodies. They are mouse antibodies or chimeric antibodies. Among them, the humanized antibodies are expressed with the prefix hz, e.g., as hz2G10, hz39D2 and hz8G11 antibodies.

The inventors of the present disclosure have made efforts to develop a novel antibody which is capable of preventing or treating cancer (particularly, breast cancer and gastric cancer), exhibits better killing ability (or proliferation-inhibiting ability) for cancer cells which have non-reactivity (or resistance) to the trastuzumab antibody or have reduced sensitivity, and is capable of preventing or treating cancer with improved anticancer activity when co-administered with the trastuzumab antibody as compared to single administration of trastuzumab. As a result, they have developed a novel antibody which exhibits better killing ability for HER2-overexpressed cancer cells on which the trastuzumab antibody hardly acts, or exhibits improved anticancer activity when co-administered with the trastuzumab antibody, and have completed the present disclosure.

The antibody of the present disclosure or an antigen-binding fragment thereof has a specific binding ability for HER2. In particular, among the antibodies of the present disclosure, hz2G10 and hz39D2 bind to an epitope in domain 1 of domains 1-4 of HER2, 24D3 binds to an epitope in domain 3, and 1G3 and hz8G11 bind to an epitope in domain 4, like trastuzumab, which is different from the epitope to which trastuzumab binds.

In the present disclosure, the term “trastuzumab” refers to an antibody disclosed in U.S. Pat. No. 5,821,337.

The antibody of the present disclosure has superior killing ability or proliferation-inhibiting ability for cancer cells which have non-reactivity (or resistance) to the trastuzumab antibody or have reduced sensitivity, when used either alone or in combination with trastuzumab. In the present disclosure, the terms “killing”, “proliferation-inhibiting” or “growth-inhibiting” are used interchangeable with the same meaning with regard to cancer cells.

In the present disclosure, the term “antibody” refers to an antibody specific for HER2, and includes not only the whole antibody but also an antigen-binding fragment of the antibody molecule.

A whole antibody has two full-length light chains and two full-length heavy chains. The light chains and heavy chains are connected by disulfide bonds. The constant region of the heavy chain has gamma (γ), mu (μ), alpha (α), delta (δ) and epsilon (ε) types, and has subclasses gamma1 (γ1), gamma2 (γ2), gamma3 (γ3), gamma4 (γ4), alpha1 (α1) and alpha2 (α2). The constant region of the light chain has kappa (κ) and lambda (λ) types.

In the present disclosure, the term “antigen-binding fragment” refers to a fragment having antigen-binding ability, and includes Fab, F(ab′), F(ab′)₂, Fv, etc. Among the antibody fragments, Fab (fragment antigen-binding) has a structure having a variable region of the light and heavy chains, a constant region of the light chain and the first constant region (C_{H1}) of the heavy chain and has one antigen-binding site. Fab′ differs from Fab in that it has a hinge region including at least one cysteine residue at the C-terminus of the heavy chain CH1 domain. In the F(ab′)₂ antibody, a cysteine residue in the hinge region of Fab′ forms a disulfide bond. Recombinant techniques for generating Fv fragments with minimal antibody fragments in which Fv has only the heavy chain variable region and the light chain variable region are known in the related art. A double-chain variable fragment (dcFv) is linked to a heavy chain variable region and a light chain variable region via a non-covalent bond, and a single-chin variable fragment (scFv) is generally linked to covalently to the variable region of a heavy chain via a peptide linker, or to the C-terminus, to form a dimer such as the double-chain Fv. These antibody fragments can be obtained using proteases (for example, Fab can be obtained by cleaving a whole antibody with papain, and the F(ab′)2 fragment can be obtained by cleaving with pepsin), or can be prepared using genetic recombination techniques.

Specifically, in the present disclosure, the antibody includes a monoclonal antibody, a multispecific antibody, a human antibody, a humanized antibody, a chimeric antibody, a single-chain Fv (scFv), a single-chain antibody, an Fab fragment, an F(ab′) fragment, a disulfide-linked Fv (dsFv), an anti-idiotypic (anti-Id) antibody, and epitope-binding fragments of these antibodies, although not being limited thereto.

In the present disclosure, the term “heavy chain” encompasses a full-length heavy chain including a variable region domain VH and three constant region domains C_{H1}, C_{H2} and C_{H3}, including an amino acid sequence having a variable region sequence sufficient for conferring specificity to an antigen, and fragments thereof. Also, in the present disclosure, the term “light chain” encompasses a full-length light chain including a variable region domain V_{L} and a constant region domain C_{L}, including an amino acid sequence having a variable region sequence sufficient for conferring specificity to an antigen, and fragments thereof.

In the present disclosure, the term “variable region” or “variable domain” refers to a domain of an antibody heavy chain or light chain associated with binding of an antibody to an antigen. In general, the variable domains of a heavy chain and a light chain (V_{H} and V_{L}, respectively) of a native antibody have similar structures, and each domain includes four conserved framework region (FRs) and three hypervariable regions (HVRs) (Kindt et al., Kuby Immunology, 6th ed., W.H. Freeman and Co., page 91 (2007)).

In the present disclosure, the term “CDR (complementarity-determining region)” refers to the amino acid sequence of the hypervariable region of a heavy chain and a light chain of an immunoglobulin (Kabat et al., Sequences of Proteins of Immunological Interest, 4th ed., U.S. Department of Health and Human Services, National Institutes of Health (1987)). Each of the heavy chain (CDRH1, CDRH2 and CDRH3) and the light chain (CDRL1, CDRL2 and CDRL3) includes three CDRs. The CDR provides major contact residue for binding of an antibody to an antigen or an epitope.

In the present disclosure, the term “framework region” or “FR” refers to a variable domain residue other than a hypervariable region (HVR) residue. The FR of a variable domain generally consists of four FR domains: FR1, FR2, FR3 and FR4. Accordingly, the HVR and FR sequences generally appear in the following order in V_{H}:
FRH1 (framework region 1 of heavy chain)-CDRH1 (complementarity-determining region 1 of heavy chain)-FRH2-CDRH2-FRH3-CDRH3-FRH4.

And, the HVR and FR sequences generally appear in the following order in V_{L} (or Vₖ):
FRL1 (framework region 1 of light chain)-CDRL1 (complementarity-determining region 1 of light chain)-FRL2-CDRL2-FRL3-CDRL3-FRL4.

In the present disclosure, the term “specific binding” means that an antibody or an antigen-binding fragment thereof, or another construct such as scFv forms a relatively stable complex with an antigen under physiological conditions. The specific binding may be characterized by an equilibrium dissociation constant of about 1×10⁻⁶ M or smaller (e.g., the smaller the K_{d}, the tighter the binding). Methods for determining if two molecules bind specifically are well known in the art, for example, equilibrium dialysis, surface plasmon resonance, etc.

In the present disclosure, the term “affinity” refers to the strength of the sum of noncovalent interactions between a single binding site of a molecule (e.g., an antibody) and its binding partner (e.g., an antigen). Unless specified otherwise, “binding affinity” refers to intrinsic binding affinity which reflects a 1:1 interaction between the members of a binding pair (e.g., an antibody and an antigen). The affinity of a molecule X for its partner Y may generally be represented by a dissociation constant (K_{d}). The affinity can be measured by common methods known in the art, including those described in the present disclosure.

In the present disclosure, the “human antibody” or “humanized antibody” possesses an amino acid sequence which corresponds to an antibody produced by human or a human cell, or an antibody derived from a non-human source that utilizes human antibody repertoires or other human antibody-encoding sequences.

In the present disclosure, the term “chimeric antibody” refers to an antibody in which a portion of the heavy chain and/or light chain is derived from a particular source or species while the remainder of the heavy chain and/or light chain is derived from a different source or species.

The anti-HER2 antibody of the present disclosure or an antigen-binding fragment thereof may include variants of the amino acid sequences described in the attached sequence listings within the scope of specifically recognizing HER2. For example, the amino acid sequence of an antibody may be modified to improve the binding affinity and/or other biological properties of the antibody. Such modification includes, for example, deletion, insertion and/or substitution of the amino acid sequence residue of the antibody.

Such amino acid variation is made based on the relative similarity of amino acid side chain substituents, such as hydrophobicity, hydrophilicity, charge, size, etc. From analysis of the size, shape and type of amino acid side chain substituents, it is recognized that arginine, lysine and histidine are positively charged residues; alanine, glycine and serine have similar sizes; and phenylalanine, tryptophan and tyrosine have similar shapes. Based on these considerations, it is thus recognized that arginine, lysine and histidine; alanine, glycine and serine; and phenylalanine, tryptophan and tyrosine are biologically functional equivalents.

For introduction of mutation, the hydropathy indices of amino acids may be considered. Each amino acid is assigned a hydropathy index according to its hydrophobicity and charge: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The hydropathy indices of amino acids are very important in imparting the interactive biological function of proteins. It is well known that similar biological activity can be retained when substitution is made with an amino acid having a similar hydropathy index. In this regard, when mutation is introduced, substitution is made between amino acids showing difference in the hydropathy index preferably within ±2, more preferably within ±1, even more preferably within ±0.5.

Meanwhile, it is also well known that substitution between amino acids having similar hydrophilicity values leads to proteins with equivalent biological activity. As disclosed in U.S. Pat. No. 4,554,101, the following hydrophilicity values are assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In this regard, when mutation is introduced, substitution is made between amino acids showing difference in the hydrophilicity value preferably within ±2, more preferably within ±1, even more preferably within ±0.5.

Amino acid substitutions in proteins that do not entirely alter the activity of the molecules are known in the art (H. Neurath, R. L. Hill, The Proteins, Academic Press, New York, 1979). The most commonly occurring substitutions are substitutions between the following amino acid residues: Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu and Asp/Gly.

In an exemplary embodiment of the present disclosure, the heavy chain variable region of the hz2G10 antibody and the 2G10 antibody respectively includes amino acid sequences of SEQ ID NO 31 and 32.

In an exemplary embodiment of the present disclosure, the light chain variable region of the hz2G10 antibody and the 2G10 antibody respectively includes amino acid sequences of SEQ ID NO 35 and 77.

In an exemplary embodiment of the present disclosure, the heavy chain variable region of the hz39D2 antibody and the 39D2 antibody respectively includes amino acid sequences of SEQ ID NO 39 and 83.

In an exemplary embodiment of the present disclosure, the light chain variable region of the hz39D2 antibody and the 39D2 antibody respectively includes amino acid sequences of SEQ ID NO 43 and 85.

In an exemplary embodiment of the present disclosure, the heavy chain variable region of the 24D3 antibody includes an amino acid sequence of SEQ ID NO 47.

In an exemplary embodiment of the present disclosure, the light chain variable region of the 24D3 antibody includes an amino acid sequence of SEQ ID NO 51.

In an exemplary embodiment of the present disclosure, the heavy chain variable region of the 1G3 antibody includes an amino acid sequence of SEQ ID NO 55.

In an exemplary embodiment of the present disclosure, the light chain variable region of the 1G3 antibody includes an amino acid sequence of SEQ ID NO 59.

In an exemplary embodiment of the present disclosure, the heavy chain variable region of the hz8G11 antibody and the 8G11 antibody respectively includes amino acid sequences of SEQ ID NO 63 and 79.

In an exemplary embodiment of the present disclosure, the light chain variable region of the hz8G11 antibody and the 8G11 antibody respectively includes amino acid sequences of SEQ ID NO 67 and 81.

The antibody of the present disclosure includes a monoclonal antibody, a multispecific antibody, a human antibody, a humanized antibody, a chimeric antibody, a single-chain Fv (scFv), a single-chain antibody, an Fab fragment, an F(ab′) fragment, a disulfide-linked Fv (dsFv), an anti-idiotypic (anti-Id) antibody, and epitope-binding fragments of these antibodies, although not being limited thereto.

Meanwhile, the antibody of the present disclosure is unique in that its CDR sequence has very low homology (similarity) to the CDR sequences of existing anti-HER2 antibodies. For example, as a result of BLAST search for hz2G10 from among the antibodies of the present disclosure, the highest CDR sequence homology of the antibody of the present disclosure to the antibodies disclosed in U.S. Pat. Nos. 8,314,213 and 8,404,811 was less than 50%. In addition, the antibodies disclosed in U.S. Pat. Nos. 8,314,213 and 8,404,811 bind to CD25 and EGFL7, respectively, and are different from the antibody of the present disclosure in their targets.

In addition, the anti-HER2 antibody of the present disclosure or an antigen-binding fragment thereof encompasses an anti-HER2 antibody including a slight change in the amino acid sequence described above, including the modification that hardly affects the tertiary structure and function of the antibody, or an antigen-binding fragment thereof. Accordingly, in some exemplary embodiments, the antibody may have an amino acid sequence with at least 90%, 93%, 95% or 98% similarity to the above-described sequence.

Also, in the present disclosure, the heavy chain variable region and the light chain variable region of the antibody or an antigen-binding fragment thereof may be linked by a linker composed of an amino acid sequence represented by the general formula (GₙSₘ)ₚ or (SₘGₙ)ₚ.

In the formula, n, m and p satisfy the followings:
n is an integer from 1 to 7;
m is an integer from 0 to 7;
n+m is an integer which is 8 or smaller; and
p is an integer from 1 to 7.

In a specific exemplary embodiment of the present disclosure, n=1-5 and m=0-5. In a more specific exemplary embodiment, n=4 and m=1. In a further more specific exemplary embodiment, the linker is (G₄S)₃ or (S₄G)₃.

In another exemplary embodiment, the linker is VDGS. In another exemplary embodiment, the linker is ASGS.

In addition, the light chain variable region and the heavy chain variable region of the antibody according to the present disclosure or an antigen-binding fragment may in the following orientations:
light chain variable region-linker-heavy chain variable region; or
heavy chain variable region-linker-light chain variable region.

Another aspect of the present disclosure provides a fusion protein including an anti-HER2 antibody or an antigen-binding fragment thereof.

In the present disclosure, the fusion protein is prepared for the productivity purification efficiency, improved biological activity, increased stability, improved folding and/or binding to a functional moiety for additional function of the anti-HER2 antibody of the present disclosure or an antigen-binding fragment thereof. The fusion protein may be formed as two or more polypeptide chains are linked by a covalent bond, or may be in the form of a conjugate wherein two or more polypeptide chains are linked by chemical conjugation.

Another aspect of the present disclosure provides a chimeric antigen receptor polypeptide including the followings:
(a) an HER2-binding domain;
(b) a transmembrane domain (TM);
(c) a costimulatory domain (domain); and
(d) an intracellular signaling domain (ICD).

In the present disclosure, the term “chimeric antigen receptor (CAR)” refers to an artificially constructed hybrid protein (fusion protein) or polypeptide containing a target-binding domain (e.g. single-chain variable fragment (scFv)) linked to an effector cell-signaling or effector cell-activating domain (e.g. T-cell signaling or T-cell activating domain). In general, the chimeric antigen receptor has the ability of redirecting T-cell specificity and reactivity toward a selected target in a non-MHC restricted manner by taking advantage of the antigen-binding property of a monoclonal antibody. The non-MHC-restricted antigen recognition confers the ability to recognize an antigen on T-cells expressing CAR, thus bypassing the major mechanism of tumor escape. Moreover, when expressed in T-cells, the CAR advantageously does not dimerize with the endogenous T-cell receptor (TCR) alpha and beta chains.

In an exemplary embodiment of the present disclosure, the chimeric antigen receptor of the present disclosure recognizes the HER2 antigen and is expressed on the cell surface since it includes the HER2-binding domain including the anti-HER2 antibody of the present disclosure or an antigen-binding fragment thereof.

The chimeric antigen receptor of the present disclosure includes a transmembrane domain because it is expressed on the cell surface. The transmembrane domain may be a transmembrane domain of a protein selected from a group consisting of the T-cell receptor alpha, beta or zeta chain, CD28, CD3 epsilon, CD45, CD4, CD5, CD8, CD9, CD16, CD22, CD33, CD37, CD64, CD80, CD86, CD134, CD137 and CD154, although not being limited thereto.

In a specific exemplary embodiment of the present disclosure, the transmembrane domain may be a transmembrane domain of CD8 or CD28.

The costimulatory domain of the chimeric antigen receptor of the present disclosure may be a functional signaling domain obtained from a protein selected from a group consisting of MHC class I molecule, TNF receptor protein, immunoglobulin-like protein, cytokine receptor, integrin, signaling lymphocytic activation molecule (SLAM), activating NK cell receptor, BTLA (B- and T-lymphocyte attenuator), Toll-like ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, and a ligand binding specifically to CD83, although not being limited thereto.

In a specific exemplary embodiment of the present disclosure, the costimulatory domain may be a functional signaling domain obtained from a protein selected from a group consisting of CD28, OX40, 4-1BB (CD137) and/or ICOS (CD278), more specifically a functional signaling domain of CD28 and/or OX40.

In another exemplary embodiment of the present disclosure, the intracellular signaling domain is a functional signaling domain of 4-1BB, CD28, OX40 or CD3 zeta, or a combination thereof. Most specifically, the intracellular signaling domain is a functional signaling domain of CD3 zeta.

In another embodiment of the present disclosure, the intracellular signaling domain may be a functional signaling domain of OX40 ligand (OX40L). In another embodiment, the intracellular signaling domain is OX40 ligand.

The HER2-binding domain of the chimeric antigen receptor of the present disclosure is linked to the transmembrane domain by a hinge domain.

In another exemplary embodiment of the present disclosure, the hinge domain may be IgG4 hinge, CD8 hinge or IgD hinge.

Another aspect of the present disclosure provides a nucleic acid molecule encoding the anti-HER2 antibody or an antigen-binding fragment thereof, or the chimeric antigen receptor polypeptide described above.

In the present disclosure, the term “nucleic acid molecule” encompasses DNA (gDNA and cDNA) and RNA molecules, and the nucleotides that are the basic building blocks of the nucleic acid molecule include not only natural nucleotides but also analogues having modified sugar or base moieties (Scheit, Nucleotide Analogs, John Wiley, New York (1980); Uhlman and Peyman, Chemical Reviews, 90: 543-584 (1990)).

A nucleotide sequence encoding the antibody of the present disclosure or an antigen-binding fragment thereof, or the chimeric antigen receptor polypeptide is not limited to a specific nucleotide sequence as long as it is a nucleotide sequence encoding the amino acid sequences constituting the chimeric antigen receptor molecule.

This is because the variation in nucleotide sequences may not lead to change in protein sequences through expression. This is called codon degeneracy. Accordingly, the nucleotide sequence includes a nucleotide sequence including functionally equivalent codons, or codons encoding the same amino acid (for example, six codons encode arginine or serine due to codon degeneracy) or codons encoding a biologically equivalent amino acid.

In a specific exemplary embodiment of the present disclosure, the nucleotide sequence encoding the polypeptide constituting the heavy chain CDR, light chain CDR, heavy chain variable region, light chain variable region, heavy chain or light chain of the anti-HER2 antibody of the present disclosure or an antigen-binding fragment thereof is described in the attached sequence listings.

The nucleic acid molecule of the present disclosure, which encodes the anti-HER2 antibody or an antigen-binding fragment thereof, or the chimeric antigen receptor polypeptide, is understood to encompass a nucleotide sequence exhibiting substantial identity for the nucleotide sequence. The substantial identity means that, when the nucleotide sequence of the present disclosure is aligned to another sequence correspond to each other as much as possible and the aligned sequences are analyzed using an algorithm commonly used in the art, the nucleotide sequences exhibit at least 80% homology, more specifically at least 90% homology, most specifically at least 95% homology.

When considering the variation of biologically equivalent activity, it is understood that the nucleic acid molecule encoding the antibody of the present disclosure or an antigen-binding fragment; or the chimeric antigen receptor polypeptide encompasses a sequence exhibiting substantial identity to the sequences described in the sequence listings. The substantial identity means that, when the sequence of the present disclosure and another sequence are aligned to correspond to each other as much as possible and the aligned sequences are analyzed using an algorithm commonly used in the art, the sequences have at least 61% homology, more specifically 70% homology, further more specifically 80% homology, most specifically 90% homology. Methods of the alignment for sequence comparison are known in the art. Various methods and algorithms for the alignment are disclosed in Smith and Waterman, Adv. Appl. Math. 2: 482 (1981); Needleman and Wunsch, J. Mol. Bio. 48: 443 (1970); Pearson and Lipman, Methods in Mol. Biol. 24: 307-31 (1988); Higgins and Sharp, Gene 73: 237-44 (1988); Higgins and Sharp, CABIOS 5: 151-3 (1989); Corpet et al., Nuc. Acids Res. 16: 10881-90 (1988); Huang et al., Comp. Appl. BioSci. 8: 155-65 (1992) and Pearson et al., Meth. Mol. Biol. 24: 307-31 (1994). The NCBI's basic local alignment search tool (BLAST) (Altschul et al., J. Mol. Biol. 215: 403-10 (1990)) is accessible from the NBCI (National Center for Biotechnology Information) and on the Internet and may be used in connection with sequence analysis programs such as blastp, blastn, blastx, tblastn and tblastx. BLAST may be accessed through the BLAST webpage of the NCBI's website. The method for comparing sequence homology using such a program is available from the BLAST help page of the NCBI's website.

In connection with the claims of the application/patent, sequence identity is determined according to the Needleman and Wunsch algorithm.

Another aspect of the present disclosure provides a recombinant vector including the nucleic acid molecule.

In the present disclosure, the term “vector” includes a delivery vector and an expression vector.

In the present disclosure, the term “delivery vector” refers to a composition of a material which contains an isolated nucleic acid and can be used to deliver the isolated nucleic acid into a cell. It includes a linear polynucleotide, a polynucleotide associated with an ionic or amphiphilic compound, a plasmid and a virus, although not being limited thereto. More specifically, the delivery vector includes a self-replicating plasmid or virus. The term is also construed to include non-plasmid and non-viral compounds which facilitate transfer of nucleic acids into cells, such as, for example, polylysine compounds, liposomes, etc. Examples of the viral delivery vector include an adenoviral vector, an adeno-associated viral vector, a retroviral vector and a lentiviral vector, although not being limited thereto.

In the present disclosure, the term “expression vector” refers to a vector including a recombinant nucleotide including an expression control sequence operably linked to a nucleotide sequence to be expressed for expression of a target gene in a host cell. The expression vector includes a cis-acting element sufficient for expression and other elements for expression can be provided by a host cell or an in-vitro expression system. The expression vector includes a plasmid vector including a recombinant polynucleotide; a cosmid vector; and a viral vector such as a bacteriophage vector, an adenoviral vector, a lentiviral vector, a retroviral vector and an adeno-associated viral vector. In a specific exemplary embodiment of the present disclosure, a nucleic acid molecule encoding a switch molecule is operatively linked to a promoter of the vector of the present disclosure. In the present disclosure, the term “operatively linked” refers to functional linkage between a nucleic acid expression control sequence (e.g., a promoter, a signal sequence, or an array of a transcription factor binding site) and another nucleic acid sequence, wherein the control sequence affects the transcription and/or translation of the another nucleic acid sequence.

The recombinant vector system of the present disclosure may be constructed according to various methods known in the art. Specific methods are described in Sambrook et al., Molecular Cloning, A Laboratory Manual, Cold Spring Harbor Laboratory Press (2001), which is incorporated into the present disclosure by reference.

The vector of the present disclosure may be constructed as a vector for gene cloning, a vector for protein expression, or a vector for gene delivery. In addition, the vector of the present disclosure may be constructed by using a prokaryotic cell or a eukaryotic cell as a host cell.

For example, when the vector of the present disclosure is an expression vector and a eukaryotic cell is used as a host cell, a promoter derived from the genome of a mammalian cell (e.g., metallothionein promoter, β-actin promoter, human hemoglobin promoter and human muscle creatine promoter) or a promoter derived from a mammalian virus (e.g., adenovirus late promoter, vaccinia virus 7.5K promoter, SV40 promoter, cytomegalovirus promoter, tk promoter of HSV, mouse mammary tumor virus (MMTV) promoter, LTR promoter of HIV, Moloney virus promoter, Epstein-Barr virus (EBV) promoter and Rous sarcoma virus (RSV) promoter) may be used, and they generally have a polyadenylation sequence as a transcription termination sequence.

In an exemplary embodiment of the present disclosure, when the vector is a delivery vector, it may be a “retroviral vector”. Retrovirus provides a convenient platform for a gene delivery system. A gene selected for gene delivery may be inserted in the retroviral vector and may be packaged within a retroviral particle. Then, the recombinant retrovirus may be delivered to a target host cell in vivo or in vitro. Many retroviral vectors are known in the art. In a specific exemplary embodiment of the present disclosure, the retroviral vector may be a pMT retroviral vector which is an MLV-based retroviral vector, although not being limited thereto.

In another exemplary embodiment of the present disclosure, the vector is a lentiviral vector or an adenoviral vector.

The vector of the present disclosure may be fused with other sequences for easy purification of the polypeptide or protein expressed thereby. For example, the fused sequence may be glutathione S-transferase (Pharmacia, USA), maltose-binding protein (NEB, USA), FLAG (IBI, USA), 6× His (hexahistidine; Quiagen, USA), etc. Meanwhile, the expression vector of the present disclosure may include a selectable marker gene and/or a reporter gene for evaluating the expression of the antibody of the present disclosure or an antigen-binding fragment thereof, or a CAR polypeptide including the same. The selectable marker gene includes an antibiotic-resistant gene commonly used in the art, e.g., genes resistant to ampicillin, gentamicin, carbenicillin, chloramphenicol, streptomycin, kanamycin, geneticin, neomycin and tetracycline. The reporter gene includes luciferase, beta-galactosidase, chloramphenicol, acetyltransferase or green fluorescent protein gene.

Methods for introducing the recombinant vector of the present disclosure into a cell and expressing the same are well known in the related art. The vector may be easily introduced into a host cell, e.g., a mammalian cell, a bacterial cell, a yeast cell or an insect cell according to methods known in the art. For example, the vector may be delivered into a host cell by physical, chemical or biological means. The physical means includes calcium phosphate coprecipitation, lipofection, particle bombardment, microinjection, electroporation, etc. The chemical means includes a colloidal dispersion system, e.g., a macromolecular complex, a nanocapsule, a microsphere, a bead, and a lipid-based system including an oil-in-water emulsion, a micelle, a mixed micelle and a liposome. And, the biological means includes use of a DNA or RNA vector such as a lentiviral vector, a retroviral vector, etc. as described above.

Another aspect of the present disclosure provides a host cell transformed with the recombinant vector.

The host cell capable of cloning and expressing the vector of the present disclosure stably and continuously may be any host cell known in the art. For example, a eukaryotic host cell suitable for the vector includes a monkey kidney cell 7 (COS7), an NSO cell, an SP2/0 cell, a Chinese hamster ovary (CHO) cell, a W138 cell, a baby hamster kidney (BHK) cell, a MDCK cell, a myeloma cell, a HuT 78 cell and an HEK-293 cell, although not being limited thereto.

Another aspect of the present disclosure provides an effector cell expressing the chimeric antigen receptor (CAR) polypeptide.

In an exemplary embodiment of the present disclosure, the effector cell is selected from a group consisting of a dendritic cell, a killer dendritic cell, a mast cell, a natural killer cell, a B lymphocyte, a T lymphocyte, a macrophage and precursor cells thereof, although not being limited thereto. The T lymphocyte cell is selected from a group consisting of an inflammatory T lymphocyte, a cytotoxic T lymphocyte, a regulatory T lymphocyte or a helper T lymphocyte.

In the present disclosure, the effector cell includes a group of autologous cells or allogenic cells. That is to say, the effector cell includes a group of autologous cells or allogenic cells expressing the HER2-specific CAR polypeptide.

In another exemplary embodiment of the present disclosure, the effector cell includes a group of cells transfected or transduced with a vector including a nucleic acid molecule encoding the HER2-specific CAR polypeptide. The transfection or transduction may be achieved by various means known in the art without limitation.

Accordingly, in a specific exemplary embodiment of the present disclosure the present disclosure, the HER2-specific CAR-encoding nucleic acid molecule is delivered into an effector cell, e.g., a T lymphocyte or a natural killer cell, and transcribed into mRNA. The HER2-specific CAR polypeptide is translated from the mRNA and expressed on the surface of the effector cell.

Another aspect of the present disclosure provides a pharmaceutical composition for preventing or treating cancer, which contains: (a) a pharmaceutically effective amount of the anti-HER2 antibody of the present disclosure or the antigen-binding fragment thereof described above; and (b) a pharmaceutically acceptable carrier.

Another aspect of the present disclosure provides a pharmaceutical composition for treating cancer or an inflammatory disease, which contains an effector cell expressing the chimeric antigen receptor polypeptide described above.

The pharmaceutical composition is a pharmaceutical composition for immunotherapy, which contains an effector cell expressing the anti-HER2 antibody or an antigen-binding fragment thereof; or the chimeric antigen receptor polypeptide.

In the present disclosure, “immunotherapy” refers to treatment of cancer by activating the immune system. Immunotherapy is classified into active immunotherapy and passive immunotherapy. Active immunotherapy includes i) cancer vaccine therapy of activating the immune system by injecting cancer cells or substances produced by cancer cells into human body, and ii) immunomodulatory therapy of activating specific leukocytes by administering immunomodulatory agents such as cytokines (interferons, interleukins, etc.), growth factors, etc. Passive immunotherapy includes antibody therapy and immune cell therapy. Specifically, immune cell therapy includes dendritic cell vaccine therapy, chimeric antigen receptor T (CAR-T) cell therapy, natural killer (NK) cell therapy, cytotoxic T lymphocyte (CTL) therapy, adoptive cell transfer, etc., although not being limited thereto. In the present disclosure, the immunotherapy mainly refers to antibody therapy using the anti-HER2 antibody and immune cell therapy using the HER2-specific CAR.

The pharmaceutical composition of the present disclosure contains an effector cell expressing the anti-HER2 antibody of the present disclosure or an antigen-binding fragment thereof; the chimeric antigen receptor polypeptide; or the chimeric antigen receptor as an active ingredient. Therefore, description of the details described above will be omitted to avoid redundancy.

As demonstrated in the following examples, the anti-HER2 antibody of the present disclosure exhibits better killing ability for MCF-7 cells on which the trastuzumab antibody hardly acts. In addition, the anti-HER2 antibody of the present disclosure exhibits improved killing ability for SKBR3 breast cancer cells when co-administered with trastuzumab. Accordingly, the composition of the present disclosure is very effective for combined administration with the trastuzumab antibody for treatment of cancer and for treatment of cancer not treated with trastuzumab.

The cancer that can be prevented or treated by the composition of the present disclosure includes various cancers known in the art. For example, it includes breast cancer, ovarian cancer, gastric cancer, lung cancer, liver cancer, bile duct cancer, bronchial cancer, nasopharyngeal cancer, laryngeal cancer, pancreatic cancer, bladder cancer, kidney cancer, colorectal cancer, colon cancer, cervical cancer, brain cancer, prostate cancer, bone cancer, head and neck cancer, skin cancer, thyroid cancer, parathyroid cancer or ureteral cancer.

Specifically, the cancer that can be prevented or treated by the composition of the present disclosure is HER2-expressing cancer, more specifically HER2-expressing breast cancer or gastric cancer.

The pharmaceutically acceptable carrier contained in the pharmaceutical composition of the present disclosure is one commonly used in preparation, and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc., although not being limited thereto. The pharmaceutical composition of the present disclosure may further contain a lubricant, a wetting agent, a sweetener, a flavor, an emulsifier, a suspending agent, a preservative, etc. in addition to the above-described ingredients. Suitable pharmaceutically acceptable carriers and preparations are described in detail in Remington's Pharmaceutical Sciences (19th ed., 1995).

The pharmaceutical composition of the present disclosure may be administered orally or parenterally. For example, it may be administered intravenously, subcutaneously, intramuscularly, intraperitoneally, topically, intranasally, intrapulmonarily, intrathecally, ocularly, intradermally, transdermally, etc.

An administration dosage of the pharmaceutical composition of the present disclosure varies depending on such factors as formulation method, administration method, the age, body weight, sex of a patient, pathological condition, food, administration time, administration route, excretion rate and responsiveness. A normally trained physician can easily determine and prescribe an administration dosage for effective treatment or prevention. In a specific exemplary embodiment of the present disclosure, a daily administration dosage of the pharmaceutical composition of the present disclosure is 0.0001-100 mg/kg. In the present disclosure, the term “pharmaceutically effective amount” refers to an amount sufficient for preventing or treating cancer.

The pharmaceutical composition of the present disclosure may be formulated into a unit-dosage form or a multiple-dosage form using a pharmaceutically acceptable carrier and/or excipient according to a method that can be easily employed by those of ordinary skill in the art to which the present disclosure belongs. The formulation may be in the form of a solution in an oily or aqueous medium, a suspension, an emulsion, an extract, a pulvis, a suppository, a powder, a granule, a tablet or a capsule, and may further contain a dispersant or a stabilizer.

In an exemplary embodiment of the present disclosure, the pharmaceutical composition of the present disclosure may further contain the trastuzumab antibody.

The pharmaceutical composition of the present disclosure may further contain, in addition to the active ingredient derived above, another pharmaceutically active medication or drug, e.g., a chemotherapy agent such as asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc., a targeted therapy agent such bevacizumab, olaparib, etc., or an immune checkpoint inhibitor such as nivolumab or pembrolizumab, or may be co-administered with them.

Another aspect of the present disclosure provides a method for treating cancer, which includes a step of administering a composition containing an effector cell expressing the anti-HER2 antibody or an antigen-binding fragment thereof; or the HER2-specific chimeric antigen receptor to a subject in need of treatment.

The cancer to be treated by the therapeutic method of the present disclosure is the same as defined above with regard to the pharmaceutical composition.

In an exemplary embodiment of the present disclosure, the subject may be a mammal or human.

Since the method for treating cancer or an inflammatory disease of the present disclosure uses an effector cell expressing the antibody or an antigen-binding fragment; or the chimeric antigen receptor described above as an active ingredient, description of the details described above will be omitted to avoid redundancy.

The anti-HER2 antibody or an antigen-binding fragment thereof described above may be used for diagnosis, e.g., diagnosis of cancer.

Accordingly, another aspect of the present disclosure provides a kit for diagnosing cancer, which includes the anti-HER2 antibody of the present disclosure or an antigen-binding fragment thereof.

Since the diagnostic kit of the present disclosure includes the anti-HER2 antibody of the present disclosure or an antigen-binding fragment thereof described above and diagnoses the same disease as described above with regard to the pharmaceutical composition of the present disclosure, description of the details described above will be omitted to avoid redundancy.

Since the kit includes an antibody, it can be prepared to be suitable for various immunoassay or immunostaining applications. The immunoassay or immunostaining includes radioimmunoassay, radioimmunoprecipitation, immunoprecipitation, enzyme-linked immunosorbent assay (ELISA), capture ELISA, inhibition or competition assay, sandwich assay, flow cytometry, immunofluorescence staining and immunoaffinity purification, although not being limited thereto. Methods for the immunoassay or immunostaining are described in Enzyme Immunoassay, E. T. Maggio, ed., CRC Press, Boca Raton, Florida, 1980; Gaastra, W., Enzyme-linked immunosorbent assay (ELISA), in Methods in Molecular Biology, Vol. 1, Walker, J. M. ed., Humana Press, N J, 1984; and Ed Harlow and David Lane, Using Antibodies: A Laboratory Manual, Cold Spring Harbor Laboratory Press, 1999, which are incorporated in the present disclosure by reference.

For example, when the method of the present disclosure is carried out by radioimmunoassay, an antibody labeled with a radioisotope (e.g., C¹⁴, I¹²⁵, P³² or S³⁵) may be used to detect the HER2 protein. When the method of the present disclosure is carried out by ELISA, a specific exemplary embodiment of the present disclosure includes: (i) a step of coating a sample to be analyzed on the surface of a solid substrate; (ii) a step of reacting the sample with the anti-HER2 antibody of the present disclosure as a primary antibody; (iii) a step of reacting the resultant of the step (ii) with a secondary antibody coupled with an enzyme; and (iv) a step of measuring the activity of the enzyme.

Appropriate examples of the solid substrate are a hydrocarbon polymer (e.g., polystyrene or polypropylene), glass, a metal or a gel, most specifically, a microtiter plate.

The enzyme coupled with the secondary antibody may include an enzyme that catalyzes chromogenic reaction, fluorescence reaction, luminescent reaction or infrared reaction, although not being limited thereto. For example, it includes alkaline phosphatase, 0-galactosidase, horseradish peroxidase, luciferase and cytochrome P450. When alkaline phosphatase is used as the enzyme coupled with the secondary antibody, a chromogenic substrate such as bromochloroindolyl phosphate (BCIP), nitro blue tetrazolium (NBT), naphthol-AS-B1-phosphate and enhanced chemifluorescence (ECF) may be used as the substrate. When horseradish peroxidase is used, a substrate such as chloronaphthol, aminoethylcarbazole, diaminobenzidine, D-luciferin, lucigenin (bis-N-methylacridinium nitrate), resorufin benzyl ether, luminol, Amplex Red (10-acetyl-3,7-dihydroxyphenoxazine), HYR (p-phenylenediamine-HCl and pyrocatechol), tetramethylbenzidine (TMB), 2,2-azino-di[3-ethylbenzthiazoline sulfonate] (ABTS), o-phenylenediamine (OPD), naphthol/pyronin, glucose oxidase, t-NBT (nitro blue tetrazolium) and m-PMS (phenzaine methosulfate) may be used.

When the method of the present disclosure is carried out by capture ELISA, the method includes: (i) a step of coating the HER2 antibody as a capture antibody on the surface of a solid substrate; (ii) a step of reacting the capture antibody with a sample; (iii) a step of reacting the resultant of the step (ii) with an HER2 detection antibody conjugated with a label; and (iv) a step of measuring a signal generated from the label.

The anti-HER2 antibody of the present disclosure has a label that generates a signal that can be detected by the detection antibody. The label includes a chemical substance (e.g., biotin), an enzyme (alkaline phosphatase, 0-galactosidase, horseradish peroxidase or cytochrome P450), a radioactive substance (e.g., C¹⁴, I¹²⁵, p³² and S³⁵), a fluorescent material (e.g., fluorescein), a light-emitting material, a chemiluminescent material and a FRET (fluorescence resonance energy transfer) material, although not being limited thereto. Various labels and labeling method are described in Ed Harlow and David Lane, Using Antibodies: A Laboratory Manual, Cold Spring Harbor Laboratory Press, 1999.

In the ELISA method and the capture ELISA, the measurement of the enzyme activity or the measurement of the signal may be carried out according to various methods known in the art. The signal may be detected easily by using streptavidin when biotin is used as the label, and using luciferin when luciferase is used.

The sample to which the kit of the present disclosure can be applied includes a cell, a tissue, a tissue-derived extract, a lysate, a purification product, a blood, a plasma, a serum, a lymph or ascites, although not being limited thereto.

The antibody of the present disclosure may be used in in-vivo or in-vitro imaging. Another aspect of the present disclosure provides a composition for imaging, which contains a conjugate in which the antibody of the present disclosure is conjugated to a label generating a detectable signal.

The label generating a detectable signal includes a T1 contrast agent (e.g., a Gd chelate compound), a T2 contrast agent (e.g., a superparamagnetic material (e.g., magnetite, Fe₃O₄, γ-Fe₂O₃, manganese ferrite, cobalt ferrite and nickel ferrite)), a radioiosotope (e.g., ¹¹C, ¹⁵O, ¹³N, P³², S³⁵, ⁴⁴Sc, ⁴⁵Ti, ¹¹⁸I, ¹³⁶La, ¹⁹⁸Tl, ²⁰⁰Tl, ²⁰⁵Bi and ²⁰⁶Bi), a fluorescent material (fluorescein, phycoerythrin, rhodamine, lissamine, Cy3 and Cy5), a chemiluminescent material, a magnetic particle, a mass label or an electron-dense particle, although not being limited thereto.

### Advantageous Effects

The features and advantages of the present disclosure may be summarized as follows:
(a) The antibody of the present disclosure or an antigen-binding fragment is an antibody that specifically binds to HER2 which is highly expressed in cancer cells (particularly, breast cancer or gastric cancer cells), and binds to an epitope that is different from an epitope to which trastuzumab binds. The present disclosure provides the antibody or the antigen-binding fragment, a chimeric antigen receptor including the same, and uses thereof.
(b) The antibody of the present disclosure or an antigen-binding fragment is unique in that its CDR sequence has very low homology to the CDR sequences of existing HER2-targeting antibodies.
(c) When compared with trastuzumab, the antibody of the present disclosure exhibits better killing ability for HER2-unexpressed cancer cells which have non-reactivity (or resistance) to the trastuzumab antibody or have reduced sensitivity. In addition, when the anti-HER2 antibody of the present disclosure is administered in combination with trastuzumab, a synergistic killing ability is achieved for cancer cells on which the trastuzumab antibody acts. Therefore, a composition of the present disclosure can be used for combined administration with the trastuzumab antibody for the treatment of cancer, or for the treatment of cancer not treated with trastuzumab. In particular, when expressed in effector cells such as T lymphocytes, etc., the chimeric antigen receptor including the anti-HER2 antibody of the present disclosure or an antigen-binding fragment may be used for immune cell therapy of various HER2-related cancers.
(d) Without wishing to be bound by theory, it is considered that the antibody of the present disclosure exhibits the above-described effects since it binds to an epitope that is different from an epitope to which trastuzumab binds and inhibits HER2 in a different manner from that of trastuzumab.

### BEST MODE FOR CARRYING OUT INVENTION

Hereinafter, the present disclosure will be described in detail through examples. However, the following examples are for illustrative purposes only and it will be apparent to those of ordinary skill in the art that the scope of the present disclosure is not limited by the examples.

### EXAMPLES

### Example 1: Development of Anti-HER2 Antibody

For development of antibodies, the extracellular domain (ECD) of the HER2 protein was produced using animal cells. The DNA in which a hinge and an Fc region (CH₂—CH₃) of human IgG1 were bound to the C-terminus of ECD was cloned into pCEP4 (Invitrogen, Cat. No. V044-50) using HindIII and BamHI restriction enzymes. Then, the cloned vector was transiently transformed into FreeStyle 293F (Invitrogen, Cat. No. R790-07) cells using polyethyleneimine (Polyscience Inc., Cat. No. 23966) and then purified from the cell culture using a Protein-A Ceramic HyperD F resin (PALL, Cat No. 20078-028). The purified protein was quantitated using a protein assay dye (Bio-Rad, Cat. No. 500-0006) and its concentration and purity were investigated via Coomassie Blue staining following SDS-PAGE. 100 μg of the protein antigen was mixed with a Freund's adjuvant (Sigma, Cat. No. F5506) and then injected intraperitoneally into BALB/c mouse (Dae Han Bio). 2 weeks later, 100 μg of the antigen diluted in PBS was injected further. 3 days later, the spleen of the mouse was taken out and lymphocytes were isolated. The isolated lymphocytes were mixed with SP2/0-Ag14 myeloma cells (ATCC, Cat. No. CRL-1581) at a ratio of 5:1 and then fused using PEG-1500 (Roche, Cat. No. 783641). The fused cells (hybridoma) were selectively sorted out and cultured in a medium containing a HAT supplement (Sigma, Cat. No. H0262).

The obtained hybridoma cells were examined via ELISA to determine whether they were the cells producing an antibody that bind to the antigen. HER2-ECD-Fc or ChromPure human IgG (hIgG, Jackson Immunoresearch Lab. Inc., Cat. No. 009-000-003) was immobilized at room temperature onto a Costar 96-well plate (Corning, Cat. No. 3590) at a concentration of 1 μg/mL for 1 hour. The resultant was washed 3 times with TBS-T (0.05% Triton X-100) and then blocked at room temperature for 30 minutes with 300 μL of TBS-T/SM (2% skim milk). After washing the blocked plate 3 times and adding the hybridoma culture, the antibody was allowed to bind at 37° C. for 1 hour. After washing 3 times and then adding anti-mIgG-HRP (Pierce, Cat. No. 31439) diluted to 1:5,000 in TBS-T/SM, as a secondary antibody, the antibody was allowed to bind at 37° C. for 1 hour. After washing the resultant 3 times and adding TMB (SurModics, Cat. No. TMBC-1000-01), the mixture was allowed to develop color at room temperature for 5 minutes. Then, the color development was stopped by adding 1 N sulfuric acid (DukSan, Cat. No. 254). Absorbance was measured at 450 nm using Victor X3 (PerkinElmer, Cat. No. 2030-0030) and the antibody binding specifically to HER2-ECD-Fc was selected.

Since HER2 is a protein expressed on cell surface, it was investigated whether the developed antibody was bound to HER2-overexpressing cells via cell-based ELISA. HER2-overexpressing SKOV-3 ovary cancer cells (Korean Cell Line Bank, Cat. No. 30077) were aliquoted onto a Costar 96-well cell culture plate (Corning, Cat. No. 3595) at 10,000 cell/well and then cultured for 24 hours. On the following day, after removing the cell culture supernatant, the resultant was washed 3 times with PBS and cultured further at 37° C. for 2 hours after adding the hybridoma cell culture. After washing 3 times with TBS-T and adding goat anti-mIgG-HRP diluted in PBS/FBS (3% FBS) to 1:5,000, as a secondary antibody, the resultant was treated at room temperature for 1 hour. After washing 3 times with TBS-T, it was allowed to develop color using TMB. 61 clones showing higher absorbance than that of the SP2/0 cell culture as a negative control group were selected.

The five antibodies (hz2G10, hz39D2, 24D3, 1G3, hz8G11) finally selected from the monoclonal antibodies binding specifically to HER2 were modified to chimeric antibodies or humanized antibodies (hz). The amino acid sequences of the chimeric antibodies or humanized antibodies are described in the attached sequence listings.

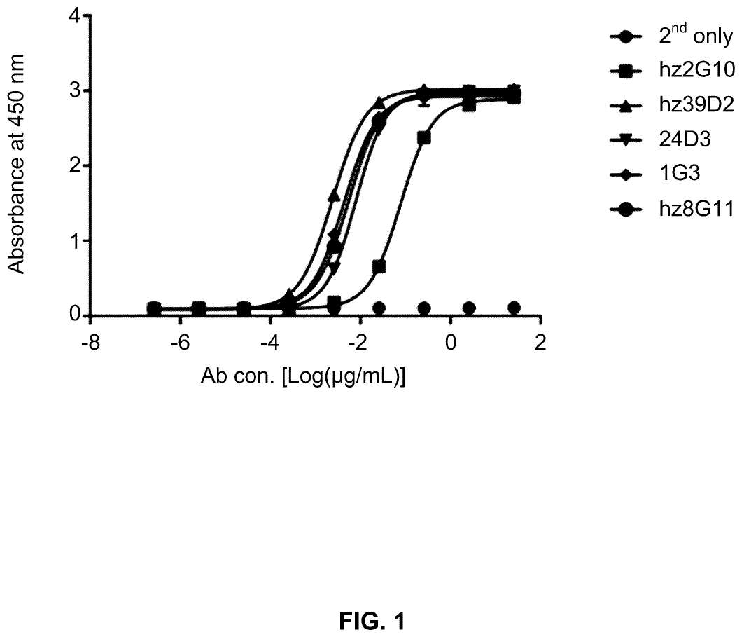

The absorbance of the finally selected five antibodies (hz2G10, hz39D2, 24D3, 1G3, hz8G11) is shown in FIG. 1 and Table 1.

### Verification of Binding of HER2 Proteins of Five Selected Antibodies to Extracellular Domain (ECD)

TABLE 1

| | Concentration (μg/mL) | | | | | | | | |
|---|---|---|---|---|---|---|---|---|---|
| Antibodies | 5 × 10⁻⁷ | 5 × 10⁻⁶ | 5 × 10⁻⁵ | 5 × 10⁻⁴ | 5 × 10⁻³ | 5 × 10⁻² | 5 × 10⁻¹ | 5 | 50 |
| PBS | 0.13 | 0.13 | 0.14 | 0.14 | 0.14 | 0.13 | 0.16 | 0.15 | 0.14 |
| hz2G10 | 0.12 | 0.12 | 0.12 | 0.12 | 0.22 | 1.16 | 2.69 | 2.79 | 2.81 |
| hz39D2 | 0.12 | 0.12 | 0.15 | 0.47 | 2.29 | 2.92 | 2.78 | 2.90 | 2.83 |
| 24D3 | 0.11 | 0.11 | 0.12 | 0.22 | 1.13 | 2.76 | 2.90 | 2.92 | 2.75 |
| 1G3 | 0.11 | 0.11 | 0.14 | 0.35 | 1.77 | 2.79 | 2.78 | 2.81 | 2.76 |
| hz8G11 | 0.12 | 0.12 | 0.14 | 0.34 | 1.67 | 2.72 | 2.94 | 2.90 | 2.74 |

### Example 2: Verification of Binding Site of Developed Antibody for HER2 Protein

The binding site of the selected five antibodies (hz2G10, hz39D2, 24D3, 1G3, hz8G11) for the extracellular domain (ECD) of the HER2 protein was verified by ELISA. For ELISA, the extracellular domain (ECD) of the ERBB family protein was produced using animal cells and was used as an antigen. Specifically, the DNA in which a hinge and an Fc region (CH₂—CH₃) of human IgG1 were bound to the C-terminus of ECD was cloned into pCEP4 (Invitrogen, Cat. No. V044-50) using HindIII and BamHI restriction enzymes. Then, the cloned vector was transiently transformed into FreeStyle 293F (Invitrogen, Cat. No. R790-07) cells using polyethyleneimine (Polyscience Inc., Cat. No. 23966) and then HER2-ECD DI Fc, HER2-ECD DII Fc, HER2-ECD DIII Fc, HER2-ECD DIV Fc and HER2-ECD Fc fusion proteins were purified from the cell culture using a Protein-A Ceramic HyperD F resin (PALL, Cat No. 20078-028). The purified protein was quantitated using a protein assay dye (Bio-Rad, Cat. No. 500-0006) and its concentration and purity were investigated via Coomassie Blue staining following SDS-PAGE.

The HER2-ECD DI Fc, HER2-ECD DII Fc, HER2-ECD DIII Fc, HER2-ECD DIV Fc and HER2-ECD Fc fusion proteins were immobilized at 4° C. overnight onto a Costar 96-well plate (Corning, Cat. No. 3590) at a concentration of 1 μg/mL for 1 hour. The resultant was washed 3 times with TBS-T (0.05% Triton X-100) and then blocked at room temperature for 1 hour with 100 μL of TBS-T/BSA (5% BSA). After washing the blocked plate 3 times and adding the anti-HER2 antibody, the antibody was allowed to bind at room temperature for 1 hour. After washing 3 times and then adding anti-human IgG-HRP diluted to 1:3,000 in TBS-T/BSA, as a secondary antibody, the antibody was allowed to bind at room temperature for 1 hour. After washing the resultant 3 times and adding TMB (SurModics, Cat. No. TMBC-1000-01), the mixture was allowed to develop color at room temperature for 5 minutes. Then, the color development was stopped by adding 1 N sulfuric acid (DukSan, Cat. No. 254). Absorbance was measured at 450 nm using Victor X3 (PerkinElmer, Cat. No. 2030-0030).

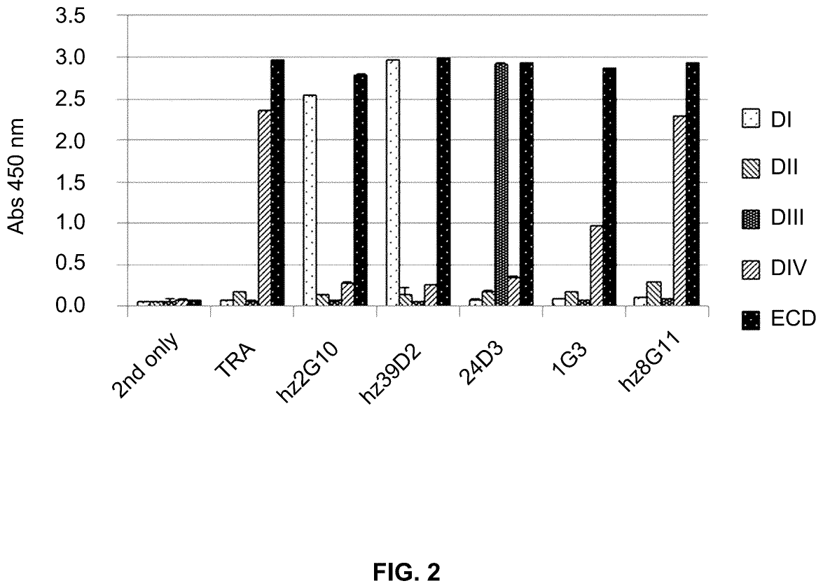

The result is shown in FIG. 2.

As show in FIG. 2, among the antibodies developed in the present disclosure, hz2G10 and hz39D2 were bound to the domain 1 of the extracellular domains of the HER2 protein, 24D3 was bound to the domain 3, and 1G3 and hz8G11 were bound to the domain 4.

From this result, it can be seen that the five antibodies of the present disclosure can inhibit the growth of the HER2-overexpressed cancer cells by binding to the HER domain which is different from the extracellular domain 4 of the HER2 protein to which trastuzumab (TRA) binds (hz2G10, hz39D2, 24D3), or can exhibit remarkably superior effect of inhibiting cellular growth when used alone or co-administered with trastuzumab. Therefore, they can be usefully used for prevention or treatment of cancer related with the expression of the HER2 proteins either alone or together with trastuzumab.

### Example 3: Comparison of Inhibitory Effect of Developed Antibody Against Growth of Breast Cancer Cells

Cell viability was analyzed by treating HER2-overexpressed SKBR3 breast cancer cells or HER2-unexpressed breast cancer cells with MCF-7 either alone or together with trastuzumab. For co-administration, the developed antibody and trastuzumab were mixed at a weight ratio of 1:1. SKBR3 cells (Korean Cell Line Bank, Cat. No. 30030, 5,000 cells/well) and MCF-7 cells (ATCC, Cat. No. HTB22, 5,000 cells/well) were aliquoted onto a 96-well plate and cultured for 24 hours. The cells were cultured further for 4 days after treating with the purified antibody at a final concentration of 20 μg/mL. For measurement of cell viability, CCK-8 (Dojindo, Cat. No. CK-04-13) was added to a final concentration of 10% and absorbance was measured after treating at 37° C. for 3 hours. Relative cell viability was calculated with respect to the absorbance of the antibody-untreated well as 100%.

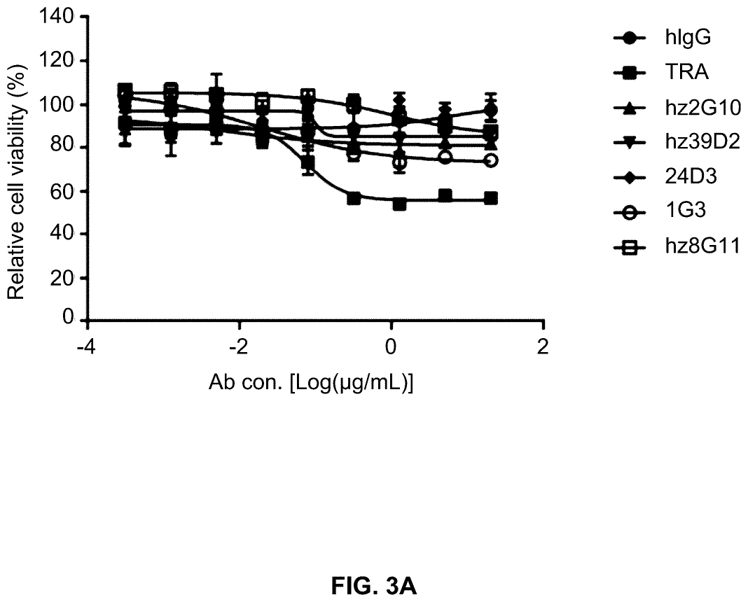

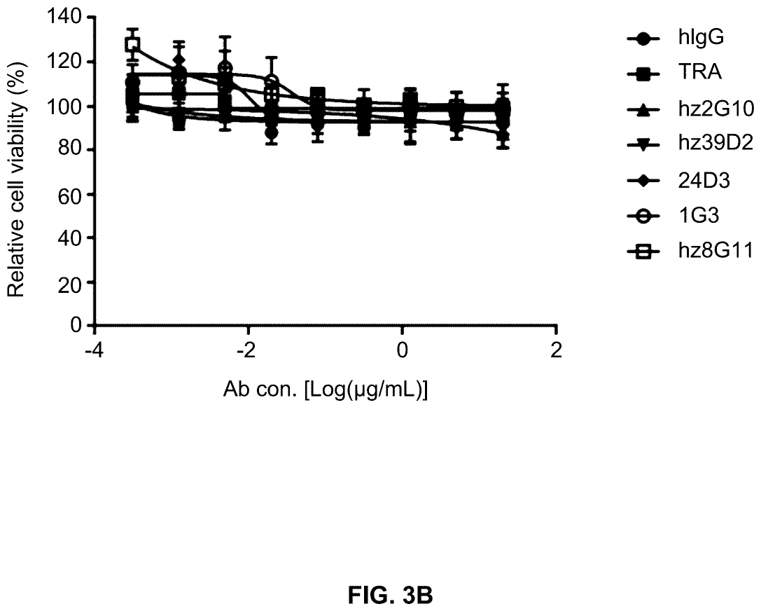

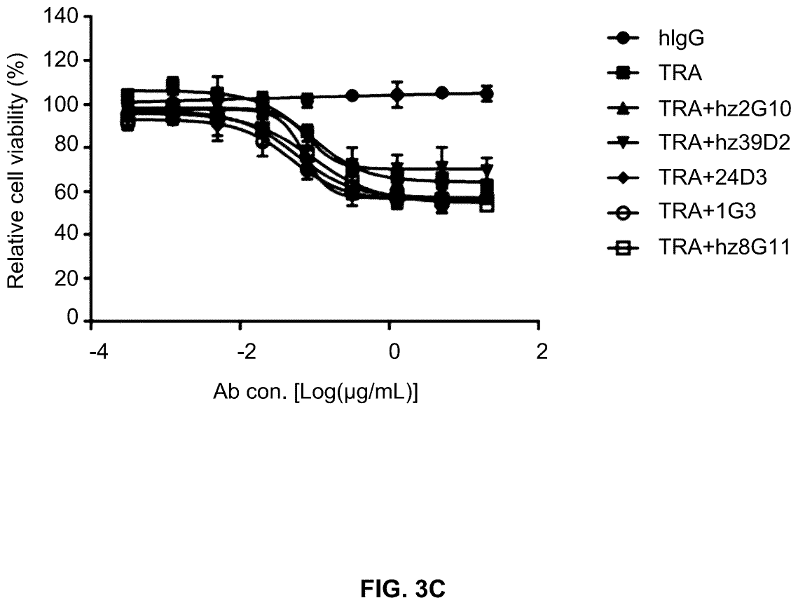

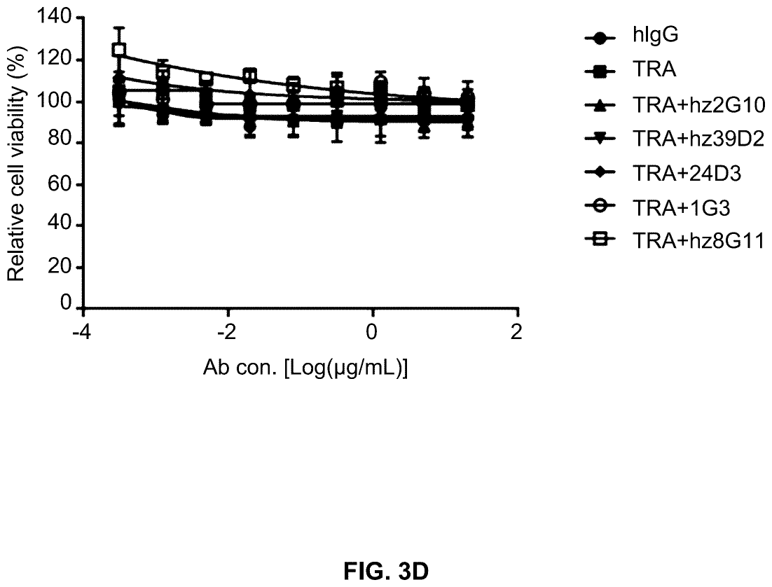

The result is shown in FIGS. 3a-3d and Table 2.

### Relative Cell Viability of HER2-Positive SKBR3 Breast Cancer Cells and HER2-Negative MCF-7 Breast Cancer Cells Treated with Antibody (Single Treatment)

TABLE 2
Relative cell viability at 20 μg/mL (%)

| Clones | SKBR3 | MCF-7 |
|---|---|---|
| hIgG | 94.68 | 92.11 |
| TRA (trastuzumab) alone | 63.68 | 98.22 |
| hz2G10 | 89.06 | 97.43 |
| hz39D2 | 96.46 | 91.42 |
| 24D3 | 93.97 | 87.33 |
| 1G3 | 74.81 | 98.66 |
| hz8G11 | 74.02 | 98.95 |

In the above table, hIgG stands for human IgG. As seen from FIG. 3A, FIG. 3B and Table 2, the five antibodies of present disclosure showed the effect of inhibiting the proliferation of SKBR3 breast cancer cells when treated alone. They showed comparable or better effect of inhibiting cellular growth as compared to the positive control trastuzumab at different concentrations (FIG. 3A). However, the five antibodies of present disclosure did not show significant effect of inhibiting cell proliferation of the HER2-negative MCF-7 cells like the positive control trastuzumab (FIG. 3B).

### Relative Cell Viability of HER2-Positive SKBR3 Breast Cancer Cells and HER2-Negative MCF-7 Breast Cancer Cells Treated with Antibody (Co-Treatment)

TABLE 3
Relative cell viability at 20 μg/mL (%)

| Clones | SKBR3 | MCF-7 |
|---|---|---|
| hIgG | 94.68 | 92.11 |
| TRA (trastuzumab) alone | 63.68 | 98.22 |
| TRA + hz2G10 | 68.98 | 90.56 |
| TRA + hz39D2 | 77.29 | 90.62 |
| TRA + 24D3 | 63.75 | 97.21 |
| TRA + 1G3 | 62.16 | 102.33 |
| TRA + hz8G11 | 52.62 | 98.41 |

In the above table, hIgG denotes the test group treated with human IgG, and TRA + denotes the test groups co-administered with trastuzumab and the antibody of the present disclosure. As seen from FIG. 3C, FIG. 3D and Table 3, all of the five antibodies of present disclosure (hz2G10, hz39D2, 24D3, 1G3, hz8G11) showed comparable or better effect of inhibiting cellular growth when treated to the SKBR3 breast cancer cells together with trastuzumab as compared to single treatment with trastuzumab (FIG. 3C).

Without wishing to be bound by theory, it is considered that the antibody of the present disclosure exhibits the above-described effect since it binds to an epitope on HER2 that is different from an epitope to which trastuzumab binds and inhibits HER2 in a different manner from that of trastuzumab.

### Example 4: Antibody Sequence Analysis

For analysis of the antibody sequence, a phage Fab antibody library was constructed using the respective hybridoma RNAs and a 3-step panning was conducted to obtain a phage that binds to HER2-ECD-Fc (Phage display: a laboratory manual, Carlos Barbas III, et al., Cold Spring Harbor Laboratory Press). After culturing the hybridoma, RNA was isolated using the SV total RNA isolation system (Promega, Cat. No. Z3100) and cDNA was synthesized therefrom. Using a known primer set (see Phage display: a laboratory manual, Carlos Barbas III, et al., Cold Spring Harbor Laboratory Press), the variable region of the antibody was amplified and cloned into the pComb3X vector (Barbas Laboratory, Scripps Research Institute) using a Sfi-I restriction enzyme after ligating to human Ck (kappa chain) and CH1, and then transformed into ER2537 bacteria (New England Biolabs, Cat. No. 801-N). The transformed bacteria were transfected with the VCSM13 helper phage (Stratagene, Cat. No. 200251) and a clone which binds to HER2-ECD-Fc was obtained using an immunotube to which HER2-ECD-Fc was immobilized.

From the colonies of the antibodies, the antibody that binds to HER2-ECD-Fc was confirmed via ELISA. The colonies of the transformed bacteria were cultured at 37° C. until the absorbance at 600 nm reached 0.5, treated with IPTG at a final concentration of 1 mM, and allowed to express antibodies in the form of Fab while culturing overnight at 30° C. After collecting cells by centrifuging 5 mL of the culture, the cells were suspended in 0.4 mL of 1×TES (50 mM Tris, 1 mM EDTA, 20% (v/v) sucrose, pH 8.0) and then treated at 4° C. for 10 minutes. After adding 0.6 mL of 0.2×TES thereto and treating further at 4° C. for 30 minutes, the resultant was centrifuged and a supernatant was taken. After washing a Costar 96-well half area plate (Corning Inc., Cat. No. 3690) coated with HER2-ECD-Fc at a concentration of 1 μg/mL 3 times with TBS-T, it was blocked with TBS-T/SM (3% non-fat skim milk, 0.05% Triton X-100) at room temperature for 1 hour. The culture broth or periplasmic extract (periplasm) of each colony was diluted at a ratio of 1:3 using TBS-T/SM and allowed to bind at room temperature for 1 hour. After washing 3 times and diluting to 1:5000 with anti-HA-HRP (Roche, Cat. No. 120-138-190-01) as a secondary antibody, the resultant was allowed to bind at room temperature for 1 hour. After washing 3 times, the resultant was allowed to develop color using TMB.

Most colonies showed absorbance of 0.2 or higher in the cell culture or periplasmic extract, and the base sequence of the antibody was analyzed for these clones. The base sequence analysis revealed that the colonies derived from the single hybridoma had the same sequences.

The CDR sequence of the antibody produced from each clone is summarized in Table 4 and Table 5.

TABLE 4

| Clones | CDRH1 | CDRH2 | CDRH3 |
|---|---|---|---|
| hz2G10 | DYYMY | YINSGGG | EALYDYD |
| | (SEQ ID | STYYPDT | YAMDY |
| | NO 1) | VKG | (SEQ ID |
| | | (SEQ ID | NO 3) |
| | | NO 2) | |
| hz39D2 | NYGVN | WINTHTGEP | DDYYVRV |
| | (SEQ ID | TYAEEFKG | DY |
| | NO 7) | (SEQ ID | (SEQ ID |
| | | NO 8) | NO 9) |
| 24D3 | SCAMS | TISGGGS | HGGYESW |
| | (SEQ ID | YTYYPDS | FPY |
| | NO 13) | VKG | (SEQ ID |
| | | (SEQ ID | NO 15) |
| | | NO 14) | |
| 1G3 | DTYMH | RID | YYYGFYA |
| | (SEQ ID | PANGYTR | MDY |
| | NO 19) | YDPNFQG | (SEQ ID |
| | | (SEQ ID | NO 21) |
| | | NO 20) | |
| hz8G11 | GYYMH | HINPNNG | EEAFAY |
| | (SEQ ID | GTSYNQK | (SEQ ID |
| | NO 25) | FKG | NO 27) |
| | | (SEQ ID | |
| | | NO 26) | |

TABLE 5

| Clones | CDRL1 | CDRL2 | CDRL3 |
|---|---|---|---|
| hz2G10 | KSSQSLL | LVSKLDS | VQGTHFP |
| | YSNGKTY | (SEQ ID | LT |
| | LN | NO 5) | (SEQ ID |
| | (SEQ ID | | NO 6) |
| | NO 4) | | |
| hz39D2 | KASQDIN | RANRLVD | LQYDEFP |
| | SYLS | (SEQ ID | WT |
| | (SEQ ID | NO 11) | (SEQ ID |
| | NO 10) | | NO 12) |
| 24D3 | RSSQSLV | KVSNRFS | SQSTHVP |
| | HSNGNTY | (SEQ ID | PWT |
| | LH | NO 17) | (SEQ ID |
| | (SEQ ID | | NO 18) |
| | NO 16) | | |
| 1G3 | KASQDVS | SASYRYT | QQHYSTP |
| | TAVA | (SEQ ID | PT |
| | (SEQ ID | NO 23) | (SEQ ID |
| | NO 22) | | NO 24) |
| hz8G11 | RASQDIS | YTSRLHS | QQGITPP |
| | NYLN | (SEQ ID | WT |
| | (SEQ ID | NO 29) | (SEQ ID |
| | NO 28) | | NO 30) |

Tables 4 and 5 show the amino acid sequences of the heavy chain CDR (CDRH) and the light chain CDR (CDRL) of the developed antibodies.

### Example 5: Specificity of Developed Antibody for HER2

It was investigated whether the developed five antibodies of the present disclosure specifically bind to HER2 belonging to the ErbB family proteins by ELISA. In order to confirm whether the developed antibody binds specifically to HER2 belonging to the ErbB family proteins, the extracellular domains of EGFR, HER2, HER3 and HER4 belonging to the ErbB family were examined via ELISA. The extracellular domain of EGFR (EGFR-ECD-Fc) was produced in the same manner as the HER2-ECD-Fc described above in Example 2, and the HER3 (R&D Systems, #348-RB-050) and HER4 (R&D Systems, #1131-ER-050) proteins were purchased. Cetuximab (CET), trastuzumab (TRA) and patritumab (AMG888, AMG) were used as control group antibodies binding to EGFR, HER2 and HER3, respectively.

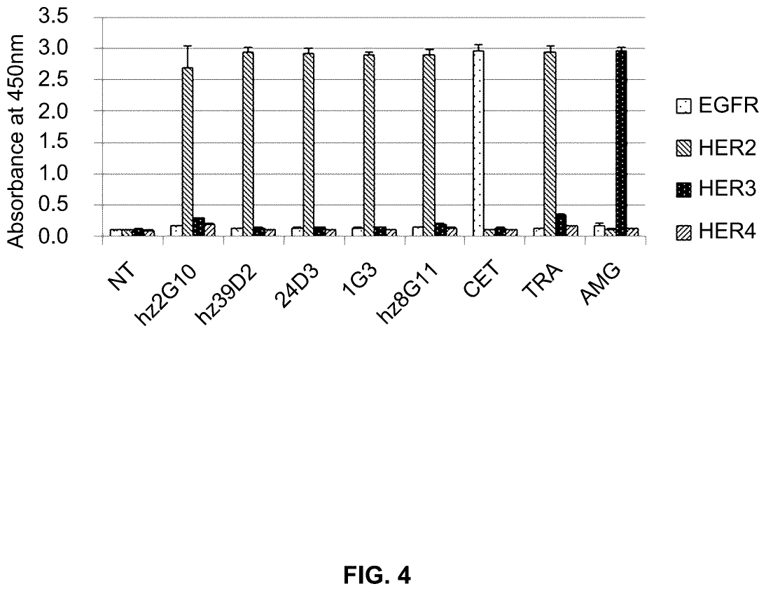

The result is shown in FIG. 4.

As seen from FIG. 4, it was confirmed that the five antibodies of the present disclosure bind specifically to HER2 among the human ErbB family proteins.

### Example 6: Comparison of Epitopes of Developed Antibody and Trastuzumab

It is known that the anti-HER2 antibody trastuzumab binds to the domain 4 among the four domains of HER2 ECD. In order to investigate whether the developed antibodies and trastuzumab share the epitope for HER2, epitope binning was conducted using Octet (Pall ForteBio). Trastuzumab was immobilized onto an AR2G sensor chip (ForteBio, Cat. Nos. 18-5092 (tray), 18-5093 (pack), 18-5094 (case)) at a concentration of 10 μg/mL by amine coupling using ECD/NHS. After allowing the HER2-ECD-His protein to bind to the trastuzumab-immobilized sensor chip at a concentration of 10 g/mL for 10 minutes, the binding between trastuzumab and HER2-ECD was stabilized for 5 minutes. Then, the five antibodies of the present disclosure were bound at a concentration of 10 μg/mL for 10 minutes and the binding between the antigen and the antibodies was stabilized for 10 minutes. After the immobilization of trastuzumab, all the antibodies and antigen were diluted using a kinetics buffer (ForteBio, Cat No. 18-1092). The same buffer was used during the stabilization. If the additionally added antibody binds to the trastuzumab-bound HER2-ECD protein, it can be interpreted that the antibody does not share the epitope with trastuzumab.

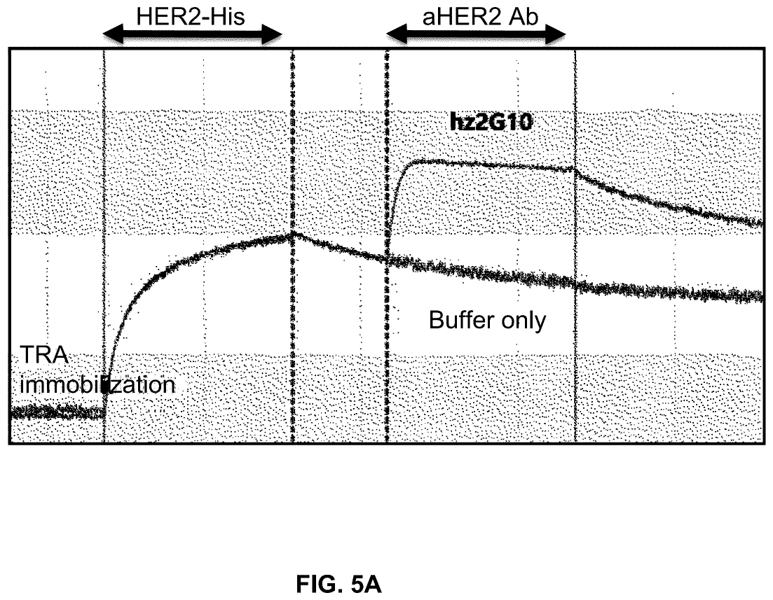

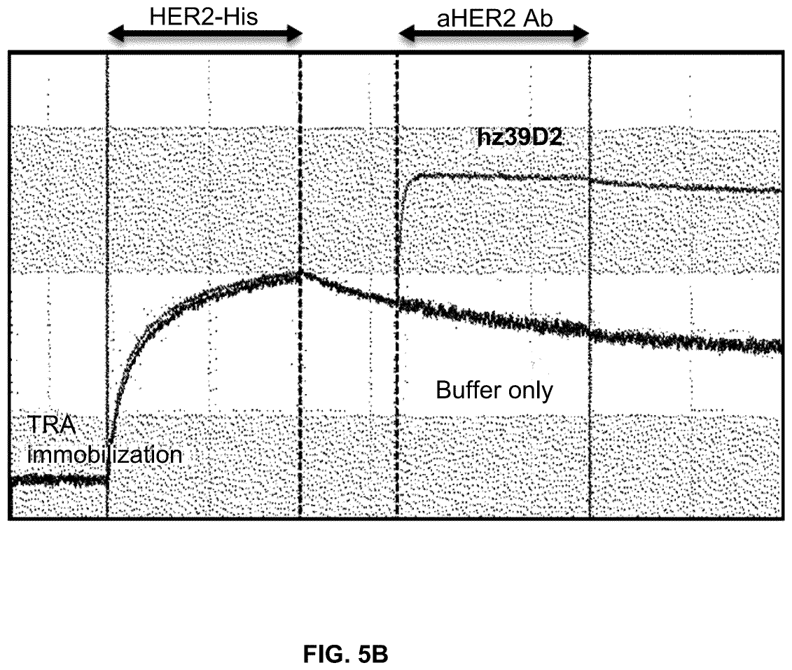

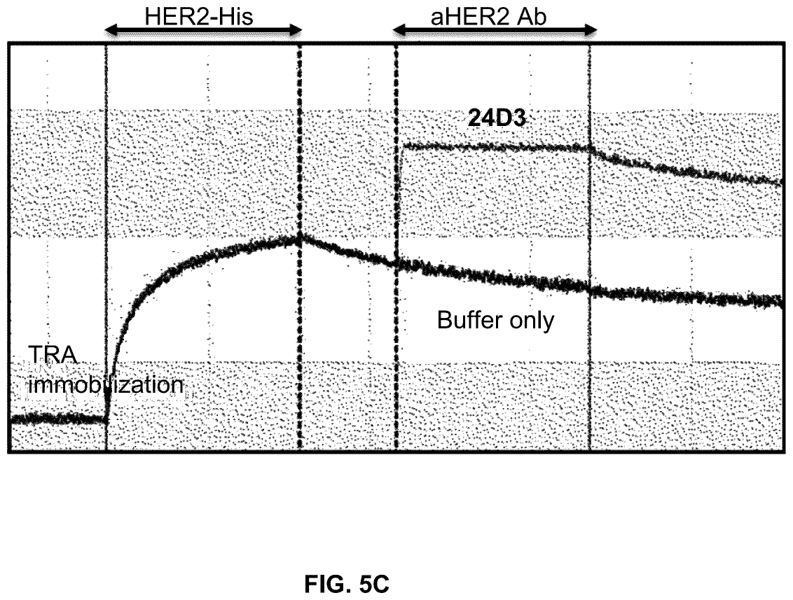

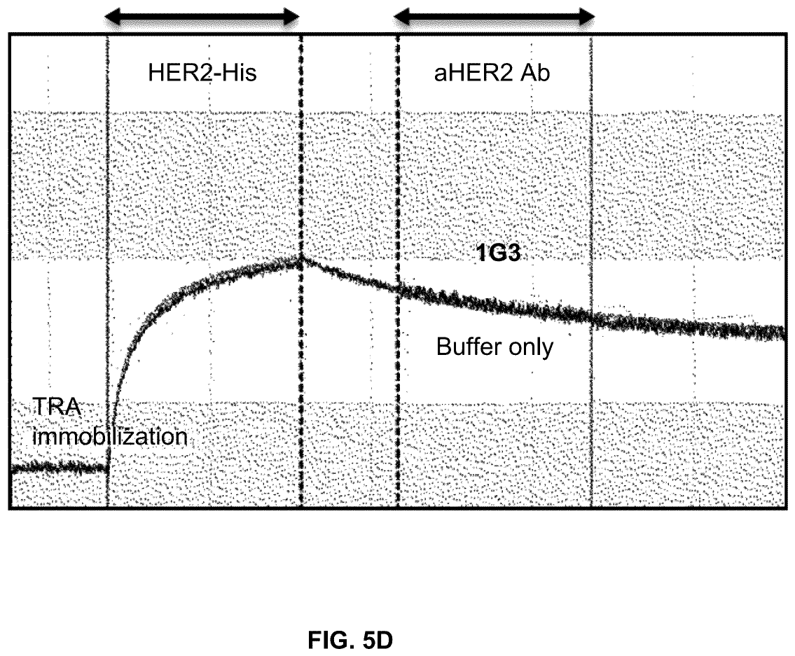

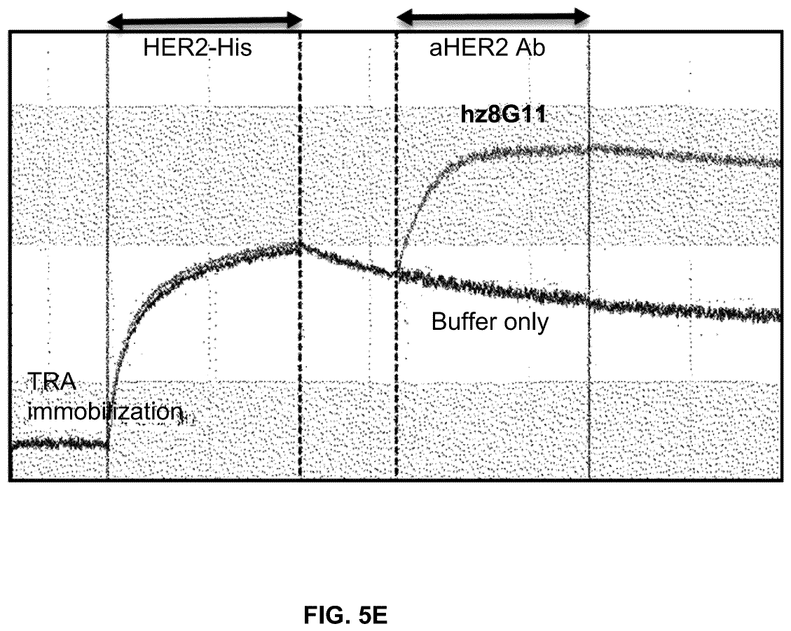

The result is shown in FIGS. 5a-5e.

As seen from FIGS. 5a-5e, it was confirmed that the developed antibodies hz2G10, hz39D2, 24D3 and hz8G11 had different epitopes from that of trastuzumab because they were bound to the trastuzumab-bound HER2-ECD. In contrast, 1G3 did not bind to the trastuzumab-bound HER2-ECD, suggesting that it shares the epitope with trastuzumab.

### Example 7: Development of hz39D2 Antibody with Increased Affinity

In order to develop antibodies with improved affinity based on the humanized 39D2 antibody (hz39D2), the inventors of the present disclosure have developed a phage antibody library with CDR3 of the light chain or heavy chain randomized (Phage display: a laboratory manual, Carlos Barbas III, et al., Cold Spring Harbor Laboratory Press). D and Y corresponding to D101 and Y102 of the CDR3 of the heavy chain according to Kabat numbering and P, W and T corresponding to P95, W96, T97 of the CDR3 of the light chain according to Kabat numbering were excluded from the randomization because they are commonly observed amino acids in human antibodies. Primers were synthesized such that adenine (A), cytosine (C), guanine (G) and thymine (T) were inserted randomly into the first and second positions of the codon corresponding to the amino acid to be randomized, with the same ratio, and guanine (G) or cytosine (C) was inserted into the third position at the same ratio. From the developed library, the clones with improved affinity were selected through biopanning using the HER2-ECD-His protein. The CDR sequence data of the finally selected three antibodies, hz39D2.14, hz39D2.22 and hz39D2.23, are summarized in Table 6 and Table 7. The amino acid residues modified to improve affinity are underlined.

TABLE 6

| Clones | CDRH1 | CDRH2 | CDRH3 |
|---|---|---|---|
| hz39D2 | NYGVN | WINTHTGEP | DDYYVRV |
| | (SEQ ID | TYAEEFKG | DY |
| | NO 7) | (SEQ ID | (SEQ ID |
| | | NO 8) | NO 9) |
| hz39D2.14 | NYGVN | WINTHTGEP | DEYYVRT |
| | (SEQ ID | TYAEEFKG | DY |
| | NO 7) | (SEQ ID | (SEQ ID |
| | | NO 8) | NO 71) |
| hz39D2.22 | NYGVN | WINTHTGEP | DEYYVRV |
| | (SEQ ID | TYAEEFKG | DY |
| | NO 7) | (SEQ ID | (SEQ ID |
| | | NO 8) | NO 72) |
| hz39D2.23 | NYGVN | WINTHTGEP | DEYYVRV |
| | (SEQ ID | TYAEEFKG | DY |
| | NO 7) | (SEQ ID | (SEQ ID |
| | | NO 8) | NO 73) |

TABLE 7

| Clones | CDRL1 | CDRL2 | CDRL3 |
|---|---|---|---|
| hz39D2 | KASQDIN | RANR | LQYDEFP |
| | SYLS | LVD | WT |
| | (SEQ ID | (SEQ ID | (SEQ ID |
| | NO 10) | NO 11) | NO 12) |
| hz39D2.14 | KASQDIN | RANR | LQYDEFP |
| | SYLS | LVD | WT |
| | (SEQ ID | (SEQ ID | (SEQ ID |
| | NO 10) | NO 11) | NO 12) |
| hz39D2.22 | KASQDIN | RANR | LELDEFP |
| | SYLS | LVD | WT |
| | (SEQ ID | (SEQ ID | (SEQ ID |
| | NO 10) | NO 11) | NO 73) |
| hz39D2.23 | KASQDIN | RANR | LQLDEFP |
| | SYLS | LVD | WT |
| | (SEQ ID | (SEQ ID | (SEQ ID |
| | NO 10) | NO 11) | NO 74) |

IgG antibodies were produced to verify the increased affinity of the three selected antibodies (hz39D2.14, hz39D2.22 and hz39D2.23). 2000 RU of goat anti-human IgG (Invitrogen, #H10500) was immobilized onto a CM5 sensor chip by ECD/NHS. Then, the antibodies were allowed to bind at a rate of 50 μL/min for 5 minutes and then stabilized for 5 minutes by flowing a buffer. After stabilizing the antibodies, the HER2-ECD-His protein was allowed to bind at a rate of 50 μL/min for 4 minutes and then separated by flowing a buffer for 15 minutes. After analyzing the concentration, the resultant was recycled using 10 mM glycine (pH 1.5) and then subjected to the subsequent assay. The affinity of the antibodies was analyzed using the BIAevaluation software. The analysis result is summarized in Table 8. As seen from Table 8, all of the three selected antibodies (hz39D2.14, hz39D2.22 and hz39D2.23) showed improved affinity as compared to hz39D2.

TABLE 8

| Clones | Kₐ (1/Ms) | K_{d} (1/s) | K_{D} (M) |
|---|---|---|---|
| hz39D2 | 6.8E+04 | 2.5E−03 | 3.7E−08 |
| hz39D2.14 | 3.7E+04 | 3.0E−04 | 8.0E−09 |
| hz39D2.22 | 8.1E+04 | 1.6E−04 | 2.0E−09 |
| hz39D2.23 | 7.1E+04 | 2.0E−04 | 2.8E−09 |

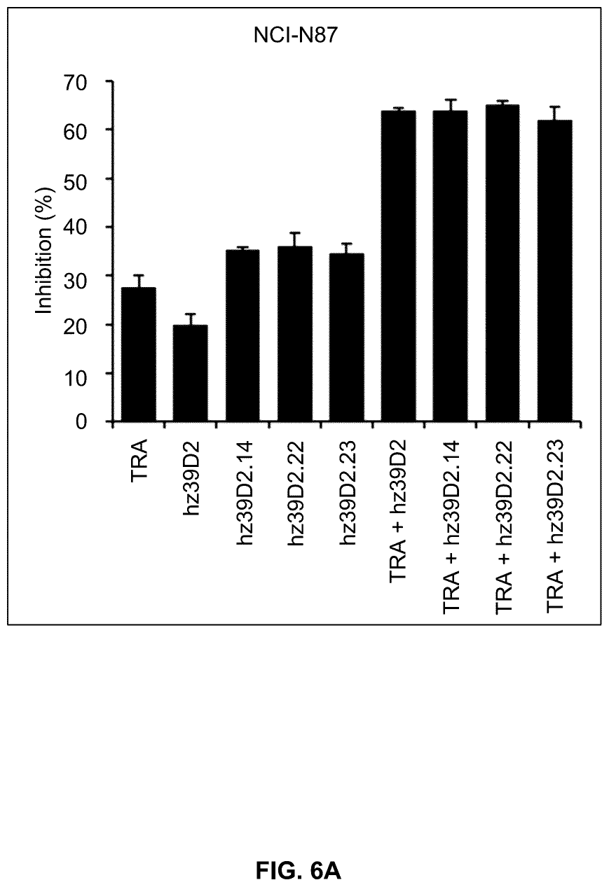

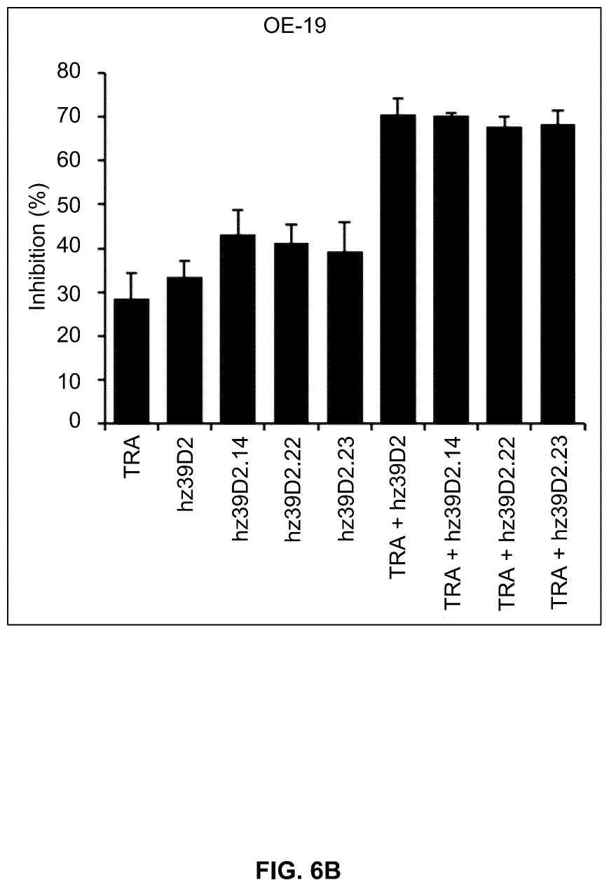

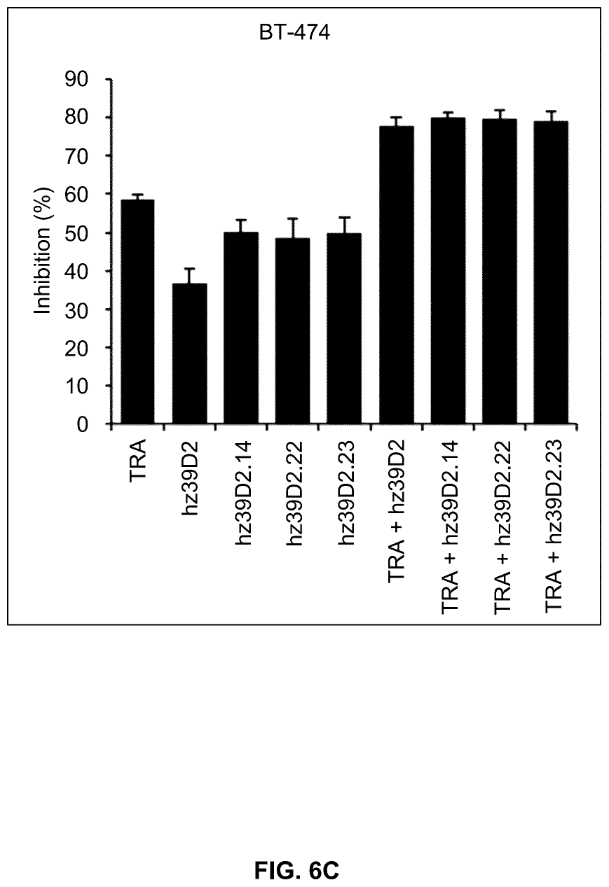

The anticancer effect of the three antibodies (hz39D2.14, hz39D2.22 and hz39D2.23) with improved affinity was analyzed using HER2-overexpressed NCI-N87 and OE-19 gastric cancer cells and BT-474 breast cancer cells. After treating the cells with each antibody at a concentration of 5 μg/mL either alone or in combination with trastuzumab, the viability of the cancer cells was analyzed (FIGS. 6a-6c). As seen from FIGS. 6a-6c, it was confirmed that the hz39D2.14, hz39D2.22, and hz39D2.23 antibodies with improved affinity showed improved effect of inhibiting cancer cell proliferation when treated alone.

### Example 8: Construction of Chimeric Antigen Receptors (CAR) Targeting Human HER2

Various CAR domain structures were constructed, cloned into lentivral expression systems, expressed in Umbilical Cord Blood-derived Natural Killer cells and tested in assays of tumor cell engagement and cytotoxic killing. The Anti-Her2 antibody disclosed herein was constructed as a scFv structure and used in combination with various CAR costimulatory domains as described herein.

Plasmid Construction: A signal sequence comprising the scFv of Her2 specific hz39D2 (VL-GS linker-VH), a hinge and transmembrane domain of CD8a, and intracellular domains of 4-1BB, OX40, OX40 ligand, and CD3 (were each independently synthesized. These molecules were assembled in various combinations using splicing by overlap extension PCR (SOE-PCR). The sequences of the PCR products were confirmed by direct sequencing. Each PCR product was cut into Nhe1 and EcoRI, and then inserted into Nhe1 and EcoRI sites of a 3rd generation self-inactivating lentiviral expression vector such as MSCV-EF1α-GFP vector or EF1a-MCS vector.

Her2-Z CAR (Clone #2) (SEQ ID NO: 129) was produced by connecting: the signal sequence domain of CD8α (nucleotides 890-952, GenBank NM 001768.6, SEQ ID NO: 112); the extracellular domain of Her2 specific hz39D2 scFv (VL-GS linker-VH) (SEQ ID NO: 114); human CD8α-derived hinge and transmembrane domains (nucleotides 1292-1507, GenBank NM 001768.6, SEQ ID NO: 116 and SEQ ID NO: 118); CD3ζ-derived intracellular signaling domain (nucleotides 299-634, GenBank NM 000734.3, SEQ ID NO: 122); and stop codon TGA.

Her2-BBZ CAR (Clone #3) (SEQ ID NO: 131) was produced by connecting: the signal sequence domain of CD8α (nucleotides 890-952, GenBank NM 001768.6, SEQ ID NO: 112); the extracellular domain of Her2 specific hz39D2 scFv (VL-GS linker-VH) (SEQ ID NO: 114); human CD8α-derived hinge and transmembrane domains (nucleotides 1292-1507, GenBank NM 001768.6, SEQ ID NO: 116 and SEQ ID NO: 118); CD137 (4-1BB)-derived intracellular signaling domain (nucleotides 901-1026, GenBank NM 001561.5, SEQ ID NO: 124); CD3ζ-derived intracellular signaling domain (nucleotides 299-634, GenBank NM 000734.3, SEQ ID NO: 122; and stop codon TGA.

Her2-28Z CAR (Clone #6) (SEQ ID NO: 133) was produced by connecting the signal sequence domain of CD8α (nucleotides 890-952, GenBank NM 001768.6, SEQ ID NO: 112); the extracellular domain of Her2 specific hz39D2 scFv (VL-GS linker-VH) (SEQ ID NO: 114); human CD8α-derived hinge domain (nucleotides 1292-1435, GenBank NM 001768.6, SEQ ID NO: 116; CD28-derived transmembrane and intracellular signaling domains (nucleotides 679-882, GenBank NM 006139.3, SEQ ID NO: 120 and SEQ ID NO: 126); CD3ζ-derived intracellular signaling domain (nucleotides 299-634, GenBank NM 000734.3, SEQ ID NO: 122); and stop codon TGA.

Her2-28OX40LZ CAR (Clone #14) (SEQ ID NO: 135) was produced by connecting: the signal sequence domain of CD8α (nucleotides 890-952, GenBank NM 001768.6, SEQ ID NO: 112); the extracellular domain of Her2 specific hz39D2 scFv (VL-GS linker-VH) (SEQ ID NO: 114); CD8α-derived hinge domain (nucleotides 1292-1435, GenBank NM 001768.6, SEQ ID NO: 116); CD28-derived transmembrane and intracellular signaling domains (nucleotides 679-882, GenBank NM 006139.3, SEQ ID NO: 120 and SEQ ID NO: 126); CD252 (OX40 ligand)-derived intracellular signaling domain (nucleotides 141-206, GenBank NM 003326.4, SEQ ID NO: 128); CD3ζ-derived intracellular signaling domain (nucleotides 299-634, GenBank NM 000734.3, SEQ ID NO: 122); and stop codon TGA.

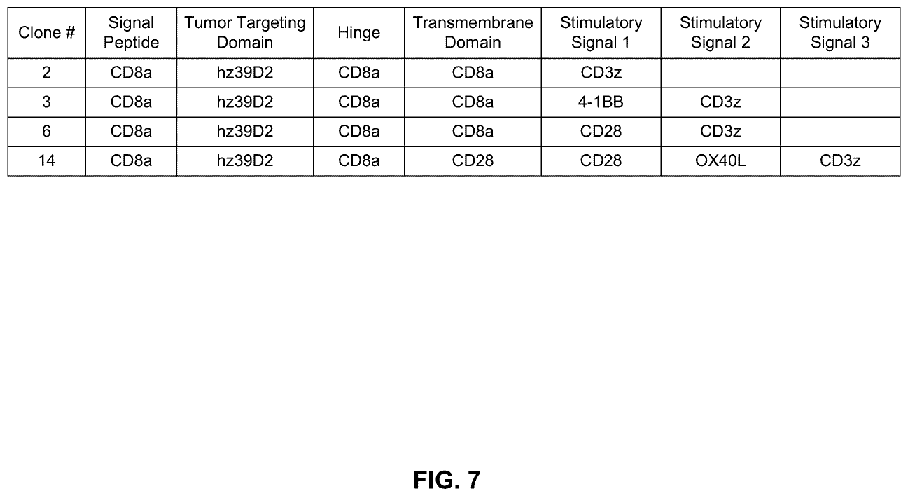

The structures of the HER2 CAR constructs as disclosed herein are summarized in FIG. 7. The domains of the CARs disclosed herein were linked in series (in tandem) to one another and linked in frame. Virus Production and Gene Transfer: To prepare VSVG-pseudotyped lentivirus, 293T cells cultured in a DMEM medium were co-transfected with various types of vectors such as PCDH1-MSCV-Her2 specific hz39D2 scFv-construct-EF1-copGFP vector, EF1a-Her2 specific hz39D2 scFv-construct vector, PCDH1-MSCV-EF1-copGFP control vector, or EF1a-GFP control vector (for production of Mock infection virus using empty vector) together with HIV-based pPACKH1 lentivirus Package Kit (System Biosciences). For this purpose, Lipofectamine 2000 reagent (Invitrogen, Carlsbad, CA) was used. Each lentivirus was prepared by transfection of 80% dense HEK293T cells in a flask with the various Her2 specific hz39D2 scFv construct expression vectors or a control plasmid together with pPACKH1 lentivirus packaging plasmids. After 6 hours, the medium was replaced by a DMEM medium containing 10% FBS. The conditioned medium containing lentivirus was collected after 48 hours of transfection, followed by filtering with a 0.45 m filter unit (Milliopore, Billerica, MA, USA) in order to remove cell debris. A viral supernatant containing the virus was concentrated about 50 times by centrifugation at 3000 rpm and 4° C. for 20 minutes using Amicon Filter (Millipore). The concentrated virus was stored at −80° C.

For the lentiviral infection, PBMC derived NK cells or cord blood derived NK cells in an exponential growth phase were adjusted to a concentration of 1×10⁶ cells/ml using Cellgro (Cellgenix) including 1% human plasma and 500 IU/mL interleukin-2, and then a lentiviral supernatant in 10 to 50 MOI was added in the presence of 4 μM BX795, 1:400 Lentiboost and 20 ng/ml IL-21 followed by centrifugation at 1000 g for 60 minutes. After centrifugation, the cells were left in a humidified incubator at 37° C. and 5% CO₂ conditions. After 24 hours of transduction, the culture medium was replaced: plate was centrifuged at 400 g for 5 minutes and transduction medium was aspirated. Equal volume fresh Cellgro (Cellgenix) including 1% human plasma and 500 IU/mL interleukin-2 was added for future use. Control cells were transduced with a vector only.

Expression Analysis of Anti-HER2-CAR (hz39D2 scFv): Her2 specific hz39D2 scFv CAR-transduced NK cells, the control vector-transduced NK (NK-Mock) or NK parent cells were washed twice with FACS buffer, and the washed cells were stained using 7-AAD (Beckman coulter), anti-CD3, anti-CD56, and recombinant histidine tagged human HER2 proteins (R&D systems) with PE-conjugated anti-histidine (Abcam) mAbs. An expression ratio and a mean fluorescence intensity (MFI) of the stained cells were measured using a BD LSRFortessa.

First, NK cells were gated in regard to singlet, and then gated in regard to 7AAD- and CD3−CD56+. The transduction efficiency of the Her2 specific hz39D2 scFv CAR constructs was determined by flow cytometric analysis of cells expressing CAR among CD3−CD56+ cells.

### Example 9: Cytotoxic Activity of Anti-Her2 CAR Constructs in NK Cells

Cells: The human breast cancer cell line HCC1954, SKBR3 and MDA-MB 468, ovarian cancer cell line SKOV-3, gastric cancer cell line N87 and human erythroleukemic cell line K562 were obtained from the American Type Culture Collection (ATCC) (Manassas, VA, USA). HCC1954, N87, SKOV-3 and K562 were maintained in RPMI-1640 (ATCC) (Manassas, VA) with 10% fetal bovine serum (FBS; Gibco, Grand Island, NY, USA). SKBR3 and MDA-MB 468 were maintained in DMEM (Gibco, Grand Island, NY, USA) with 10% FBS. PBMC or cord blood derived NK cells and transduced NK cells were maintained in CellGro® serum-free media+1% human plasma+500 IU/mL interleukin-2. Human embryonic kidney fibroblast 293T cells were obtained from the ATCC and maintained in DMEM (Gibco, Grand Island, NY, USA) supplemented with 10% FBS % (Gibco, Grand Island, NY, USA).

Calcein Releasing Cytotoxicity Assay: Target cells were labeled at 37° C. for 1 hour with 30 μM calcein-acetoxymethyl ester (Calcein-AM; Molecular probes). After washing, the labeled target cells were dispensed to 1×10⁴ cells per well in 96-well plates. Control or CAR transduced NK cells were harvested, washed, and then were added at different E/T (effector-to-target) ratios. After 2 hours, the plates were centrifuged at 2000 rpm for 3 minutes, and a supernatant of 100 μL was collected and subjected to measurement of calcein release using a fluorescence microplate reader (Victor3, PerkinElmer) at an excitation wavelength of 485 nm and an emission wavelength of 535 nm. Specific calcein release amount was calculated by the following equation: percent specific lysis=(test release−spontaneous release)×100/(maximal release−spontaneous release). For maximal lysis, a 1% Triton X-100 solution was used.

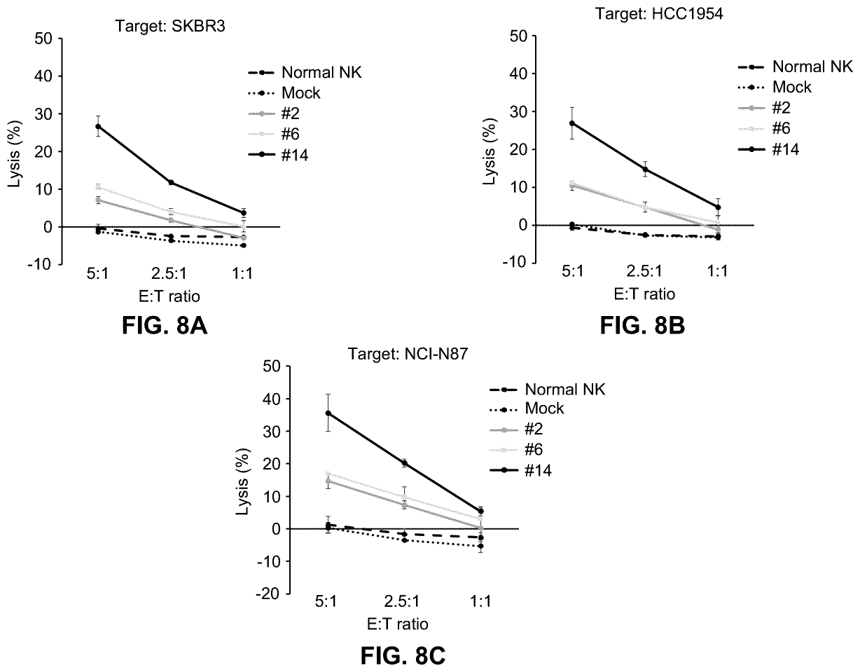

Comparison of CAR Constructs in vitro Her2+ Cell Killing: The cell killing activities of clone #2, #6 and #14 were compared against various target cells. Cord-blood derived NK (CBNK) cells were transduced with each construct using lentiviral vector system at 40 multiplicity of infection (MOI) at day 7 after culture, followed on day 11 by a positive magnetic activated cell sorting (MACS) process for HER-2-CAR-NK expressing cells. As controls, we used unmodified CBNK and mock-transduced CBNK that was transduced with lentivirus vector harboring GFP gene instead of the CAR genes. Killing assay (Calcein releasing cytotoxicity assay) was performed using HER2 positive target cancer cell lines: SKBR3 (breast cancer), HCC1954 (breast cancer, trastuzumab resistant), NCI-N87 (gastric cancer) at different ratios of effector to target cells (5:1, 2.5:1, 1:1). All of the HER-2-CAR-NKs showed higher cytotoxicity to HER-2 expressing target cells than non-transduced or mock vector transduced NK cells. Clone #14 showed unexpectedly the highest cytotoxicity to all three target cells (FIGS. 8A-8C).

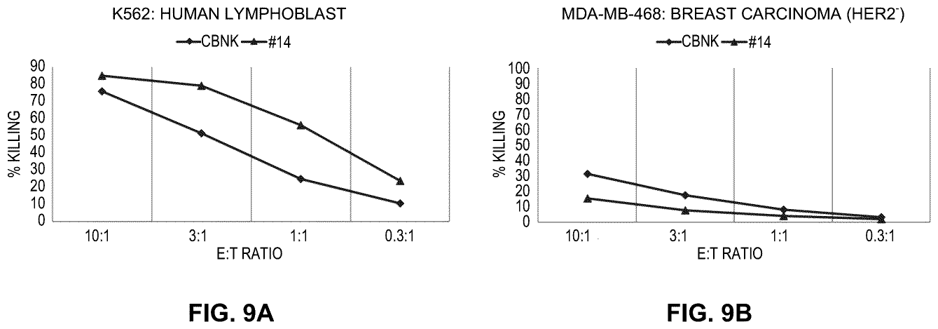

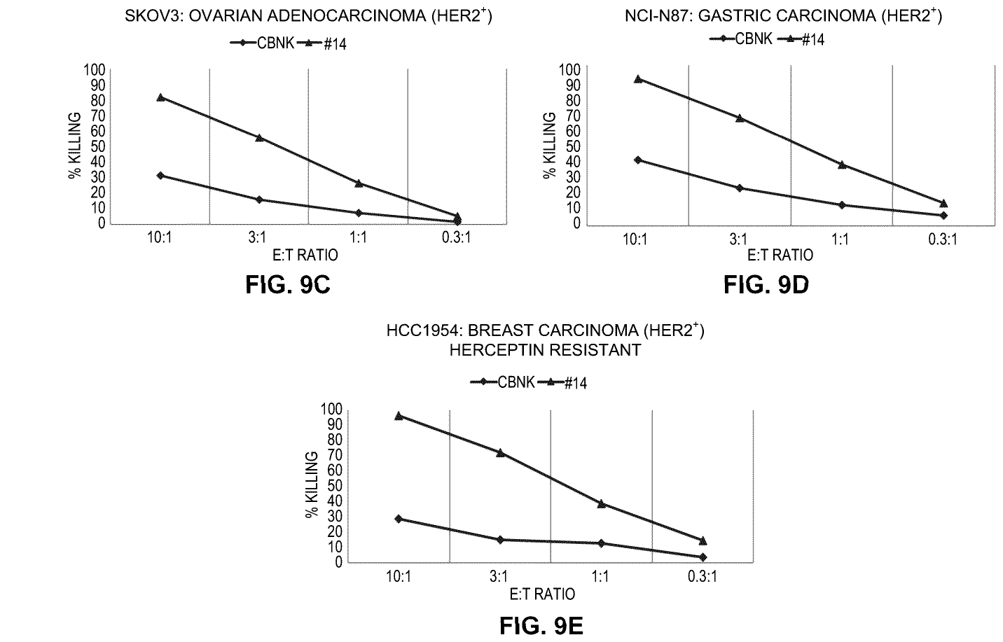

The killing activity of clone #14 was further assessed by testing its cytotoxicity to various cancer cell lines: K562 (lymphoblast, HER-2 negative, but NK susceptible control), MDA-MB-468 (breast cancer, HER-2 negative), SKOV3 (ovarian cancer, HER-2 positive), NCI-N87 (gastric cancer, HER-2 positive), and trastuzumab-resistant cell line HCC1954 (breast cancer, HER-2 positive). The cytotoxicity of clone #14 was compared with unmodified CBNK at various ratios of effector to target cells (10:1, 3:1, 1:1 & 0.3:1). Clone #14 showed significantly higher and unexpected cytotoxicity against HER2 positive cancer cell lines than unmodified CBNKs. HER2-CAR-NK was also active on HCC1954 (breast cancer, HER-2 positive), which has previously seen to be resistant to trastuzumab mAb treatment (FIGS. 9A-9E).

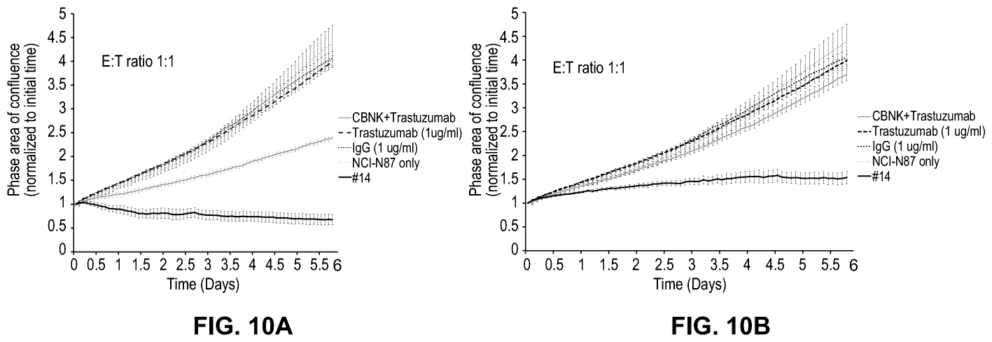

In vitro Long-Term Her2+ Cell Killing: The long-term serial killing activity of clones #6 & #14 was assessed using the Incucyte live cell imaging system. NCI-N87 Her2+ gastric carcinoma target cells were grown and monitored in the Incucyte system for 6 days in the presence of: control, non-specific IgG, trastuzumab (anti-Her2 monoclonal antibody), CBNK cells, CBNK cells in combination with trastuzumab, or CBNK transduced with clone #14 CAR construct. The experiment was completed at effector to the target (E:T) cellular ratios of 1:1 and 0.3:1. Unexpectedly, clone #14 killed significantly more Her2+ target cells than either CBNKs or CBNK in combination with tastuzumab for both the E:T 1:1 and 0.3:1 conditions (FIGS. 10A and 10B).

### Example 10: NK Cell Activation by Anti-Her2-CAR Constructs in Response to Target Cells

Intercellular Cytokine Staining (ICS) Assay for CD107a, IFN-γ, and TNF-α: To measure intracellular cytokines and CD107a of NK cells, NK cells were co-cultured with tumor targets at 1:1 ratio for 4 h in the presence of anti-CD107a-APC (H4A3; BD Biosciences, USA), GolgiStop™ and GolgiPlug™ (BD Bioscience, USA). After 4 hours, cells were washed with BD FACS flow buffer and stained with anti-CD3-FITC, anti-CD56-APC-eFluor®780, and 7-AAD permeabilized by BD CytoFix/CytoPerm™ and then stained with anti-IFN-7-PE (B27; BD Biosciences) and anti-TNF-α-PE-Cy7 (Mab11; eBioscience). Stained cells were acquired on LSR Fortessa and data analysis was conducted using FlowJo software (TreeStar Inc., OR).

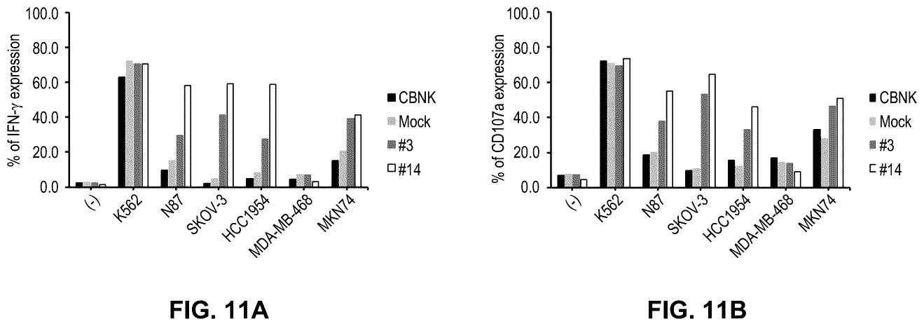

NK cell degranulation activity and cytotoxic cytokine expression with the various CAR constructs was evaluated by comparing intercellular expression level of CD107a & IFN-γ. Umbilical Cord blood-derived NK (CBNK) cells transduced with clone #3, clone #14 lentivirus were compared with control CBNK cells and CBNK cells with mock lentiviral transduction. NK cells expressing clone #3, clone #14 and control cells were co-cultured with target cancer cells: K562 (HER2-negative, but susceptible to NK cell), NCI-N87, SKOV3, HCC1954, MKN74 (HER-2 negative), and MDA-MB-468 (HER-2 negative) at 1:1 ratio of effector to target cells for 2 hrs, followed by FACS analysis gating CD56+/CD107a or CD56+/IFN-γ. Both clones #3 & #14 showed an increase in intercellular expression of CD107a & IFN-γ in response to HER-2 positive target cells. Unexpectedly, clone #14 showed consistently greater degranulation activity and IFN-γ expression than clone #3 (FIG. 11A and FIG. 11B).

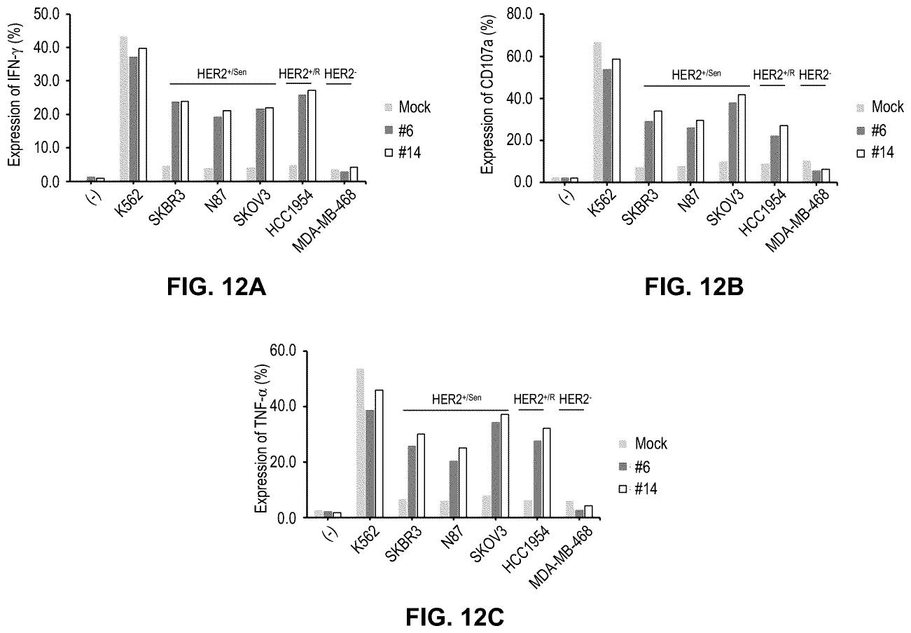

A similar assay was performed comparing Clone #6 and Clone #14 transduced CBNK cells with mock transduced CBNK cells. Again, degranulation and IFN-γ expression was unexpectedly greater in the anti-Her2 CAR transduced CBNK cells compared with control cells in response to Her2+ target cells. Further, intracellular expression of TNF-α was unexpectedly greater in the anti-Her2 CAR transduced CBNK cells compared with Control cells in response to Her2+ target cells (FIG. 12A, FIG. 12B, FIG. 12C).

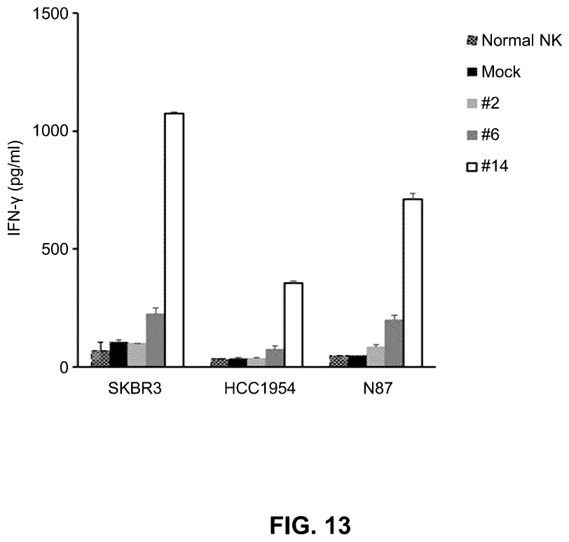

IFN-γ Secretion: The secretion of IFN-γ from anti-HER2-CAR-CBNKs (clones #2, #6, #14) was compared by co-culturing with target cells. Cell culture supernatants from the 4 hr killing assay (FIG. 8A-8C) were assayed for secreted IFN-γ using an ELISA assay. CBNK cells expressing Clone #14 unexpectedly secreted significantly more IFN-γ when co-cultured with target cancer cells than all other clones and controls. Clone #6 consistently secreted more IFN-γ in response to target cancer cells in comparison with control cells and Clone #2 (FIG. 13).

### Example 11: In Vivo Anti-Tumor Activity of Anti-Her2-CAR-NK Cells

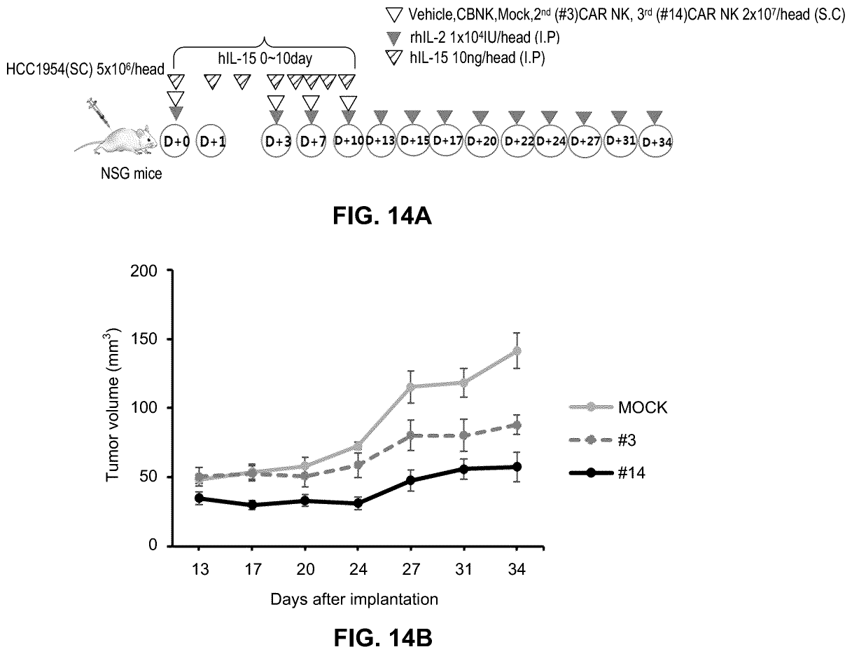

To evaluate in vivo efficacy of anti-Her2-CAR clones #3 and #14, Xenograft models in NSG mice were generated by injecting 5×10⁶ HCC1954 cells/mouse (HER-2 positive, trastuzumab resistant cells) subcutaneously at Day 0. Subsequently, 2×10⁷ cells of clone #3, #14, or mock-transduced CBNK were administered to the mice subcutaneously at day 0, 3, 7, 10. Additionally, Human IL-15 (10 ng/head) was intraperitoneally injected 8 times every 1-2 days up to day 10 and human IL-2 (1×10⁴ IU/head) was intraperitoneally injected 13 times every 2-4 days up to day 34. Tumor volume was assessed every 3-4 days after injection. Both clone #3 & #14 significantly suppressed tumor growth in mice on comparison with control, Mock-transduced CBNKs. Unexpectedly, clone #14 showed significantly better suppression of tumor growth than clone #3 (FIG. 14A and FIG. 14B).

### Example 12: Culturing and Characterization of Anti-Her2-CAR-NK Cells

NK Cell Isolation From Umbilical Cord Blood or Peripheral Blood: CD3+ cells were removed by magnetic sorting system VarioMACS (Miltenyi Biotec, Germany) for NK cell enrichment from healthy donor derived UCB or PBMCs.

Generation of Feeder Cell Lines: HuT 78 cells were transduced with 4-1BBL, mTNF-α, or mbIL-21 in combination. The 4-1BBL insert was prepared from 4-1BB expressing vector (Origene, USA) by PCR. OX40L was synthesized from Bioneer (South Korea). mbIL-21 was synthesized with the sequence of IL-21 active protein, CD8 signal peptide, CD8 hinge, and CD8 transmembrane and further codon optimized. cDNA of mTNF-α was prepared by reverse transcription-polymerase chain reaction (RT-PCR) from PBMCs. TNF-α converting enzyme (TACE) recognition site mutation was introduced by replacing Ala-Val (A-V) with Pro-Val (P-V) by site directed mutagenesis kit (Agilent Technologies, USA). Inserted genes and lentiviral vectors (SBI, USA) were digested by EcoRI and BamHI (New England BioLabs, USA) and ligated by In-Fusion HD cloning kit (Clontech, USA). Lentiviral concentrate was produced in 293T by lipofectamine 2000 (Thermofisher Scientific, USA) and concentrated by Amicon Ultra-15 Centrifugal Filter Unit with Ultracel-100 membrane (Merckmillipore, USA). 0.5×10⁶ cells/mL HuT 78 cells were suspended in 1 mL OPTU-MEM containing 50 uL lentiviral concentrate and 10 μg/mL polybrene (Santa Cruz Biotechnology, USA) and spinoculated at 1800 g, 320 for 90 minutes. HuT 78 cells transduced with lentiviral system were selected with antibiotics. 4-1BBL/mTNF-α mbIL-21 positive HuT 78 cells were isolated by flow cytometry-guided sorting (FACSMelody™ Cell Sorter, BD bioscience, USA).

Ex vivo Expansion and Cryopreservation of NK Cells: CD3+ depleted cells (1×10⁶ cells/mL) were seeded in CellGro SCGM medium (CellGenix, Germany) containing 1%-2% donor-plasma, 7-irradiated (2,000 rad) eHuT 78 (2.5×10⁶ cells/mL) in CellGro SCGM medium containing 2% donor plasma, 1000 IU/mL IL-2 and 10 ng/mL anti-CD3 monoclonal antibody OKT3. Depending on culture duration, eHuT 78 cells were stimulated every 2 weeks. Cultured cells were fed with Cellgro SCGM containing 1% donor-plasma and 1000 IU/mL IL-2 (culture medium) to maintain cell concentration of 1-2×10⁶ cells/mL for 14 days or 28 days. For cryopreservation of NK cells, harvested cells were suspended in freezing media and stored in a liquid nitrogen tank.

Immunostaining and Flow Cytometric Analysis: The following monoclonal antibodies were used to stain NK cells: anti-CD56-APC-eFluor®780 (CMSSB), and anti-CD62L-PE (DREG-56) (eBioscience, USA), anti-CD3-FITC (UCHT1), anti-CD14-FITC (M5E2) anti-CD16-PE (3G8), anti-DNAM-1-PE (DX11), anti-CD25-PE (M-A251), anti-CD44-PE (515), anti-CD56-PE-Cy5 (B159), anti-CXCR3-PE(1C6/CXCR3), anti-NKp30-PE (P30-15), anti-NKp44-PE (P44-8.1), anti-NKp46-PE (9E2/NKp46) antiOX40L-PE (ik-1), anti-4-1BBL-PE (C65-485), anti-4-1BB-PE (C65-485), anti-OX40-PE (ACT35), anti-CD27-PE (MT-271), anti-CD27L-PE (Ki-24), anti-CD30-PE (BerH8), anti-CD30L-PE, anti-CD3-PE-cy5.5 (SP34-2), anti-CD4-FITC (RPA-T4) (BD Biosciences, USA), anti-NKG2A-PE (131411), anti-NKG2C-PE (134591), anti-NKG2D-PE (149810), anti-TNF-α (membrane)-PE (6401) anti-TNF-α (membrane)-PE (6401), anti-TNFRII-PE (22235) (R&D systems, USA), anti-CD30L-PE (RM153) (Biolegend, USA). Live cells were gated with 7-AAD (Beckman-Coulter, USA). T cells and HuT 78 cells were stained with anti-TcRα/β-FITC (WT31), anti-CD2-PE (RPA-2.10), anti-CD7-FITC (4H9), anti-CD11a-FITC (G43-258), anti-CD25-PE (M-A251), anti-CD28-FITC (CD28.2), anti-CD44-PE (515) and anti-CD49d-PE (9F10) (BD Bioscience, USA). eHuT 78 cells were stained with anti-TNF-α (membrane)-PE (6401), (R&D systems, USA), antiOX40L-PE (ik-1), anti-4-1BBL-PE (C65-485) (BD bioscience, USA), and anti-IL21-PE (3A3-N2) (eBioscience, USA). Stained cells were acquired on LSR Fortessa and data were analyzed using FlowJo software (TreeStar Inc., OR).

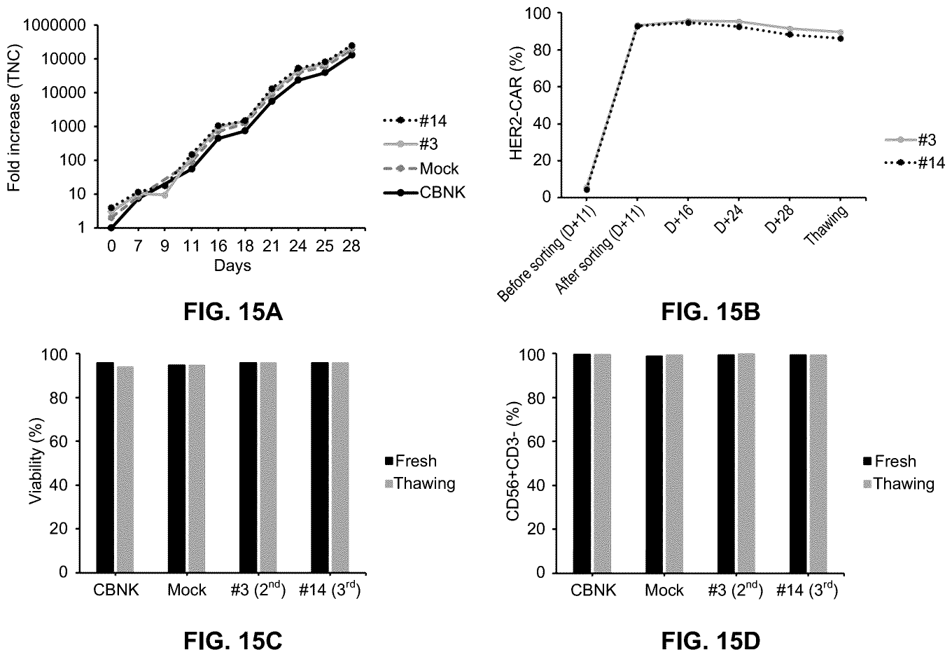

Comparison of Proliferation of HER2-CAR-NK Cells: The growth of CBNK cells, mock-transduced CBNK cells, or HER-2-CAR clones #6 and #14 was compared in culture for 28 days. During the culture period, cells were stimulated with irradiated feeder cells (eHut 78) twice at day 0 & day 14. Both clone #6 and #14 showed comparable growth pattern to unmodified and mock-transduced CBNKs (FIG. 15A).

Assessment of CAR Expression During Expansion, Culture, and Cryopreservation: To assess the CAR expression level in Lentiviral transduced CBNK cells, the HER-2 CAR population was analyzed by flow cytometry at day 11, day 16, day 24, day 28 and after freezing and thawing. Both clone #6 and #14 maintained more than 80% of CAR expression up to 28 day and through freezing and thawing (FIG. 15B).

Viability of HER2-CAR-NK Cells During Expansion, Culture, and Cryopreservation: The cell viability for HER-2CAR-NK was analyzed after 28 day-culture and freezing and thawing using propidium iodide staining-based live cell counting. Both clone #6 and #14 showed more than 95% of cell viability and no viability changes were observed in the process of freezing and thawing at 28 days after culture (FIG. 15C and FIG. 15D).

Purity of HER2-CAR-NK Cells During Expansion Culture and Cryopreservation: The cell purity for HER-2CAR-NK was analysed after expansion culture and the freezing and thawing process using flow cytometry analysis. Counts for CD56+/CD3− cells were assessed. Both clone #6 and #14 maintained near 100% of CD56+/CD3− cell population at 28 days after culture and post the process of freezing and thawing process (FIG. 15D).

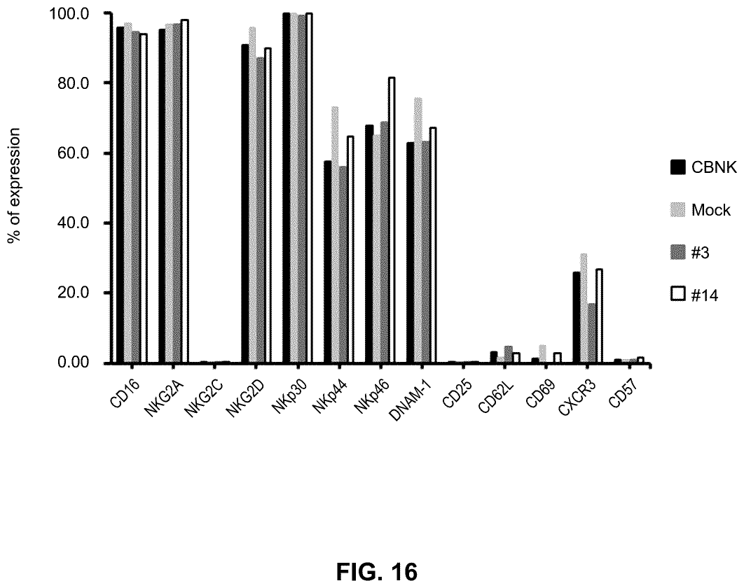

Phenotypic Cell Surface Marker Expression Analysis of HER2-CAR-NK Cells: The expression NK cell surface markers on unmodified, mock-transduced CBNK and clone #6 and #14 was assessed by flow cytometry analysis (CD16, NKG2A, NKG2C, NKG2D, NKp30, NKp44, NKp46, CD25, CD62L, CD69, CXCR3, CD57). The expression levels of all markers tested in both HER2-CAR-NK cells expressing clones #6 and #14 were comparable to those of control NK cells (FIG. 16).

What is claimed is:

1. An antibody against HER2 (human epidermal growth factor receptor 2) comprising any one of (a) to (e), or an antigen-binding fragment thereof:
(a) a heavy chain variable region comprising CDRH1 of SEQ ID NO 1, CDRH2 of SEQ ID NO 2 and CDRH3 of SEQ ID NO 3, and a light chain variable region comprising CDRL1 of SEQ ID NO 4, CDRL2 of SEQ ID NO 5 and CDRL3 of SEQ ID NO 6;
(b) a heavy chain variable region comprising CDRH1 of SEQ ID NO 7, CDRH2 of SEQ ID NO 8 and CDRH3 of SEQ ID NO 9, 71 or 72, and a light chain variable region comprising CDRL1 of SEQ ID NO 10, CDRL2 of SEQ ID NO 11 and CDRL3 of SEQ ID NO 12, 73 or 74;
(c) a heavy chain variable region comprising CDRH1 of SEQ ID NO 13, CDRH2 of SEQ ID NO 14 and CDRH3 of SEQ ID NO 15, and a light chain variable region comprising CDRL1 of SEQ ID NO 16, CDRL2 of SEQ ID NO 17 and CDRL3 of SEQ ID NO 18;
(d) a heavy chain variable region comprising CDRH1 of SEQ ID NO 19, CDRH2 of SEQ ID NO 20 and CDRH3 of SEQ ID NO 21, and a light chain variable region comprising CDRL1 of SEQ ID NO 22, CDRL2 of SEQ ID NO 23 and CDRL3 of SEQ ID NO 24; and
(e) a heavy chain variable region comprising CDRH1 of SEQ ID NO 25, CDRH2 of SEQ ID NO 26 and CDRH3 of SEQ ID NO 27, and a light chain variable region comprising CDRL1 of SEQ ID NO 28, CDRL2 of SEQ ID NO 29 and CDRL3 of SEQ ID NO 30.

2. The antibody or the antigen-binding fragment thereof according to claim 1, wherein
the heavy chain variable region of (a) comprises an amino acid sequence of SEQ ID NO 31 or 75;
the heavy chain variable region of (b) comprises an amino acid sequence of SEQ ID NO 39, 83, 87, 95 or 103;
the heavy chain variable region of (c) comprises an amino acid sequence of SEQ ID NO 47;
the heavy chain variable region of (d) comprises an amino acid sequence of SEQ ID NO 55; and
the heavy chain variable region of (e) comprises an amino acid sequence of SEQ ID NO 63 or 79.

3. The antibody or the antigen-binding fragment thereof according to claim 1, wherein
the light chain variable region of (a) comprises an amino acid sequence of SEQ ID NO 35 or 77;
the light chain variable region of (b) comprises an amino acid sequence of SEQ ID NO 43, 85, 91, 99 or 107;
the light chain variable region of (c) comprises an amino acid sequence of SEQ ID NO 51;
the light chain variable region of (d) comprises an amino acid sequence of SEQ ID NO 59; and
the light chain variable region of (e) comprises an amino acid sequence of SEQ ID NO 67 or 81.

4. The antibody or the antigen-binding fragment thereof according to claim 1, wherein
the antibody or the antigen-binding fragment thereof comprising (a) comprises a heavy chain comprising an amino acid sequence of SEQ ID NO 33;
the antibody or the antigen-binding fragment thereof comprising (b) comprises a heavy chain comprising an amino acid sequence of SEQ ID NO 41, 89, 97 or 105;
the antibody or the antigen-binding fragment thereof comprising (c) comprises a heavy chain comprising an amino acid sequence of SEQ ID NO 49;
the antibody or the antigen-binding fragment thereof comprising (d) comprises a heavy chain comprising an amino acid sequence of SEQ ID NO 57; and
the antibody or the antigen-binding fragment thereof comprising (e) comprises a heavy chain comprising an amino acid sequence of SEQ ID NO 65.

5. The antibody or the antigen-binding fragment thereof according to claim 1, wherein
the antibody or the antigen-binding fragment thereof comprising (a) comprises a light chain comprising an amino acid sequence of SEQ ID NO 37;
the antibody or the antigen-binding fragment thereof comprising (b) comprises a light chain comprising an amino acid sequence of SEQ ID NO 45, 93, 101 or 109;
the antibody or the antigen-binding fragment thereof comprising (c) comprises a light chain comprising an amino acid sequence of SEQ ID NO 53;
the antibody or the antigen-binding fragment thereof comprising (d) comprises a light chain comprising an amino acid sequence of SEQ ID NO 61; and
the antibody or the antigen-binding fragment thereof comprising (e) comprises a light chain comprising an amino acid sequence of SEQ ID NO 69.

6. A fusion protein comprising the antibody or the antigen-binding fragment thereof according to any of claims 1 to 5.

7. A chimeric antigen receptor polypeptide comprising:
(a) an HER2-binding domain;
(b) a transmembrane domain (TM);
(c) a costimulatory domain; and
(d) an intracellular signaling domain (ICD).

8. The chimeric antigen receptor polypeptide according to claim 7, wherein the HER2-binding domain comprises the antibody or the antigen-binding fragment thereof according to any of claims 1 to 5.

9. The chimeric antigen receptor polypeptide according to claim 7, wherein the transmembrane domain is a transmembrane domain of a protein selected from a group consisting of T-cell receptor alpha, beta or zeta chain, CD28, CD3 epsilon, CD45, CD4, CD5, CD8, CD9, CD16, CD22, CD33, CD37, CD64, CD80, CD86, CD134, CD137 and CD154.

10. The chimeric antigen receptor polypeptide according to claim 7, wherein the costimulatory domain is a functional signaling domain obtained from a protein selected from a group consisting of MHC class I molecule, TNF receptor protein, immunoglobulin-like protein, cytokine receptor, integrin, signaling lymphocytic activation molecule (SLAM), activating NK cell receptor, BTLA (B- and T-lymphocyte attenuator), Toll-like ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, and a ligand binding specifically to CD83.

11. The chimeric antigen receptor polypeptide according to claim 7, wherein the intracellular signaling domain comprises a functional signaling domain of 4-1BB, CD28, OX40 or CD3 zeta, or a combination thereof.

12. A nucleic acid molecule encoding the anti-HER2 antibody or the antigen-binding fragment thereof according to claim 1.

13. A nucleic acid molecule encoding the chimeric antigen receptor polypeptide according to claim 7.

14. A recombinant vector comprising the nucleic acid molecule according to claim 12 or 13.

15. A host cell transformed with the recombinant vector according to claim 14.

16. An effector cell expressing the chimeric antigen receptor polypeptide according to claim 7.

17. The effector cell according to claim 16, wherein the effector cell is selected from a group consisting of a dendritic cell, a killer dendritic cell, a mast cell, a natural killer cell, a B lymphocyte, a T lymphocyte, a macrophage and precursor cells thereof.

18. The effector cell according to claim 17, wherein the T lymphocyte is selected from a group consisting of an inflammatory T lymphocyte, a cytotoxic T lymphocyte, a regulatory T lymphocyte or a helper T lymphocyte.

19. A pharmaceutical composition for preventing or treating cancer, comprising: (a) a pharmaceutically effective amount of the anti-HER2 antibody or the antigen-binding fragment thereof according to any of claims 1 to 3; and (b) a pharmaceutically acceptable carrier.

20. A pharmaceutical composition for treating cancer, comprising the effector cell expressing the chimeric antigen receptor polypeptide according to claim 16.

21. The composition according to claim 19 or 20, wherein the cancer is breast cancer, ovarian cancer, gastric cancer, lung cancer, liver cancer, bronchial cancer, nasopharyngeal cancer, laryngeal cancer, pancreatic cancer, bladder cancer, colorectal cancer, colon cancer, cervical cancer, brain cancer, prostate cancer, bone cancer, head and neck cancer, skin cancer, thyroid cancer, parathyroid cancer or ureteral cancer.

22. The composition according to claim 19 or 20, wherein the pharmaceutical composition further comprises the trastuzumab antibody.

23. A kit for diagnosing cancer, comprising the anti-HER2 antibody or the antigen-binding fragment thereof according to any of claims 1 to 5.

24. A chimeric antigen receptor comprising an extracellular domain that binds Her2, wherein the extracellular domain comprises an amino acid sequence having at least 90% sequence identity to SEQ ID NO: 113 (hz39D2 (VL-GS linker-VH)).

25. The chimeric antigen receptor of claim 24, wherein the extracellular domain comprises an amino acid sequence having at least 95% sequence identity to SEQ ID NO: 113 (hz39D2 (VL-GS linker-VH)).

26. The chimeric antigen receptor of claim 24, wherein the extracellular domain comprises an amino acid sequence having at least 99% sequence identity to SEQ ID NO: 113 (hz39D2 (VL-GS linker-VH)).

27. The chimeric antigen receptor of claim 24, wherein the extracellular domain comprises SEQ ID NO: 113 (hz39D2 (VL-GS linker-VH)).

28. The chimeric antigen receptor of claim 24, further comprising
an extracellular signaling domain linked to the extracellular domain;
a hinge domain linked to the extracellular domain;
a transmembrane domain linked to the hinge domain; and
an intracellular stimulatory signal linked to the hinge domain.

29. The chimeric antigen receptor of claim 28, wherein the extracellular signaling domain comprises an amino acid sequence having at least 90% sequence identity with SEQ ID NO: 111 (CD8α signal peptide).

30. The chimeric antigen receptor of claim 29, wherein the extracellular signaling domain comprises SEQ ID NO: 111 (CD8α signal peptide).

31. The chimeric antigen receptor of claim 30, wherein the hinge domain comprises an amino acid sequence having at least 90% sequence identity with SEQ ID NO: 115 (CD8α hinge).

32. The chimeric antigen receptor of claim 31, wherein the hinge domain comprises SEQ ID NO: 115 (CD8α hinge).

33. The chimeric antigen receptor of claim 32, wherein the transmembrane domain comprises an amino acid sequence having at least 90% sequence identity with SEQ ID NO: 117 (CD8α TM) or SEQ ID NO: 119 (CD28 TM).

34. The chimeric antigen receptor of claim 33, wherein the transmembrane domain comprises SEQ ID NO: 117 CD8α TM) or SEQ ID NO: 119 (CD28 TM).

35. The chimeric antigen receptor of claim 34, wherein the intracellular stimulatory signal comprises an amino acid sequence having at least 90% sequence identity with SEQ ID NO: 121 (CD3-ζ).

36. The chimeric antigen receptor of claim 35, wherein the intracellular stimulatory signal comprises SEQ ID NO: 121 (CD3-ζ).

37. The chimeric antigen receptor of claim 36, further comprising a second intracellular stimulatory signal, wherein the second intracellular stimulatory signal comprises an amino acid sequence having at least 90% sequence identity with SEQ ID NO: 123 (4-1BB) or SEQ ID NO: 125 (CD28).

38. The chimeric antigen receptor of claim 37, wherein the second intracellular stimulatory signal comprises SEQ ID NO: 123 (4-1BB) or SEQ ID NO: 125 (CD28).

39. The chimeric antigen receptor of claim 38, further comprising a third intracellular stimulatory signal, wherein the third intracellular stimulatory signal comprises an amino acid sequence having at least 90% sequence identity with SEQ ID NO: 127 (OX40L).

40. The chimeric antigen receptor of claim 39, wherein the third intracellular stimulatory signal comprises SEQ ID NO: 127 (OX40L).

41. The chimeric antigen receptor of claim 28, wherein the chimeric antigen receptor comprises an amino acid sequence having at least 90% sequence identity to SEQ ID NO: 129 (Clone 2), SEQ ID NO: 131 (Clone 3), SEQ ID NO: 133 (Clone 6), or SEQ ID NO: 135 (Clone 14).

42. The chimeric antigen receptor of claim 28, wherein the chimeric antigen receptor comprises an amino acid sequence having at least 95% sequence identity to SEQ ID NO: 129 (Clone 2), SEQ ID NO: 131 (Clone 3), SEQ ID NO: 133 (Clone 6), or SEQ ID NO: 135 (Clone 14).

43. The chimeric antigen receptor of claim 28, wherein the chimeric antigen receptor comprises an amino acid sequence having at least 99% sequence identity to SEQ ID NO: 129 (Clone 2), SEQ ID NO: 131 (Clone 3), SEQ ID NO: 133 (Clone 6), or SEQ ID NO: 135 (Clone 14).

44. The chimeric antigen receptor of claim 28, wherein the chimeric antigen receptor comprises SEQ ID NO: 129 (Clone 2), SEQ ID NO: 131 (Clone 3), SEQ ID NO: 133 (Clone 6), or SEQ ID NO: 135 (Clone 14).

45. A nucleic acid molecule encoding the chimeric antigen receptor of claim 24.

46. A vector comprising the nucleic acid molecule of claim 45.

47. An immune cell expressing the chimeric antigen receptor of claim 24.

48. The immune cell of claim 47, wherein the immune cell is a natural killer cell.

49. A pharmaceutical composition comprising the immune cell of claim 48 and a pharmaceutically acceptable carrier.

50. A method for treating cancer comprising administering to a subject in need thereof a therapeutically effective amount of the pharmaceutical composition of claim 49.

51. The method of claim 50, wherein the cancer is selected from the group consisting of breast cancer, ovarian cancer, gastric cancer, lung cancer, liver cancer, bronchial cancer, nasopharyngeal cancer, laryngeal cancer, pancreatic cancer, bladder cancer, colorectal cancer, colon cancer, cervical cancer, brain cancer, prostate cancer, bone cancer, head and neck cancer, skin cancer, thyroid cancer, parathyroid cancer and ureteral cancer.

52. The chimeric antigen receptor of claim 7, wherein the intracellular signaling domain comprises a functional signaling domain of OX40 ligand.
